(19) United States
(12) Patent Application Publication
(10) Pub. No.: US 2004/0087478 A1
(43) Pub. Date: May 6, 2004

(54) Screening method

(76) Inventors: Clemens Gillen, Aachen (DE); Ingrid Wetzels, Aachen (DE); Stephan Wnendt, Aachen (DE); E. Weihe, Marburg (DE); M.K.-H. Schaefer, Marburg (DE)

Correspondence Address: CROWELL & MORING LLP, INTELLECTUAL PROPERTY GROUP, P.O. BOX 14300, WASHINGTON, DC, 20044-4300, US

(21) Appl. No.: 10/343,710

(22) PCT Filed: Aug. 3, 2001
(86) PCT No.: PCT/EP01/09011

(51) Int. Cl. C12Q 1/68; G01N 33/53; A61K 31/00

(52) U.S. Cl. 514/1; 435/6; 435/7.1

(57) ABSTRACT

The invention relates to a method for discovering pain-relevant or pain-regulating substances, associated polynucleotides, peptides, proteins, vectors and cells, to compounds identified thereby, corresponding medicaments, diagnostic reagents, and to their use in pain therapy.

The invention relates to a method for discovering pain-relevant substances, associated polynucleotides, peptides, proteins, vectors and cells, to compounds identified thereby, corresponding medicaments, diagnostic reagents, and to the use thereof in pain therapy.

Various medicaments are available for the treatment of pain such as, e.g., acetylsalicylic acid, paracetamol, dipyrone, tramadol, morphine und fentanyl, but also substances such as amitryptiline und ketamine are used for treating patients suffering from pain. Often, however, especially in chronic pain states, no lasting improvement can be achieved for patients in spite of increasingly refined treatment schemes. One of the reasons for this is the fact that in chronic pain permanent changes take place in the nerve cells involved.

Pain research in recent years has led to the fundamental recognition that plastic changes in the nervous system cause the development, of chronic pain in particular, especially changes in the nociceptive neurones of the dorsal root ganglia and the neurones in the region of the dorsal horns of the spinal chord (for an overview see: Coderre et al. 1993; Zimmermann & Herdegen, 1996). Neuronal plasticity is accompanied by changes in the expression of certain genes and leads to long-term change in the phenotype of the affected neurones. Hitherto the concept of neuronal plasticity has been applied, primarily, to developmental, learning and regeneration processes, but the most recent findings in pain research show that this concept applies also in pathophysiological processes (Tölle, 1997).

Chronification of pain is already relatively well characterised in animal experiments on a phenomenological level. The induction of chronic pain states leads to the following changes:

increased sensitivity and reduced absolute threshold of peripheral nociceptors

activation of so-called silent nociceptors

reorganisation of receptive fields

increased sensitivity in the spinal chord.

Said plastic changes have been described both for the primary afferences occurring in the ganglia and for the downstream neurones located in the spinal chord and are also suspected above the spine, e.g. in the thalamus. By analogy to the mechanisms described for learning and memory processes, it is to be assumed that a specific gene programme takes place in the cells involved comprising the coordinated regulation of relevant genes, the expression whereof then significantly contributes to the pathophysiological spread of chronic pain.

The point of departure of the invention was therefore the identification of such pain-regulated genes, the expression whereof changes in pain states and which are probably therefore involved in the emergence and processing of, in particular, chronic pain, by means of the connections in their regulation.

Regulation has already been demonstrated in various pain models for a number of known genes (see Table 1), as, for example, for neurotransmitters (substance P, CGRP), receptors (substance P receptors, μ, κ, δ opiate receptors, NMDA receptors) and transcription factors (cJun, JunB, cFos or Krox24). The fact that the above receptors are already used as molecular targets for the development of new analgesics (Dickenson, 1995), gives a clear indication that also the identification of new pain-regulated genes is of great interest for the development of analgesics, in particular for corresponding screening methods. The central idea in this context is to stop pain, in particular chronic pain, from arising or persisting, by influencing the function of those proteins the formation whereof is increased or decreased in pain states.

TABLE 1
Regulation of known genes/gene products in pain animal models

| | | | | |
|---|---|---|---|---|
| Gene (product) | Reg | Tissue/cell | Model | Literature |

(a) Neurotransmitters

| | | | |
|---|---|---|---|
| CGRP | SC-DH | UV irradiation of | Gillardon F et |
| | | the skin | al. (1992) Ann |
| | | | NY Acad |
| | | | Sci657: 493-96 |
| Preprotachykinin & | DRG | Monoarthritis | Donaldson L F |
| CGRP-mRNA | | | et al. (1992) |
| | | | Mol Brain Res |
| | | | 16: 143-49 |
| Preprotachykinin - | SC-DH | Formalin | Noguchi & |
| mRNA | | | Ruda (1992) J |
| | | | Neurosci 12: |
| | | | 2563-72 |
| Prodynorphin | SC | Exp arthritis | Höllt et al |
| mRNA | | | (1987) |
| | | | Neurosci Lett |
| | | | 96: 247-52 |
| Dynorphin Prot. | SC | Formalin | Ruda et al. |
| | | | (1988) |
| | | | PNAS 85: 622-26 |
| Substance P | Nociceptors | Exp. arthritis | Levine J D et al. |
| | | | (1984) |
| | | | Science 226: |
| | | | 547-49 |

(b) Neurotrophines

| | | | |
|---|---|---|---|
| BDNF mRNA & | DRG: trkA + cells | i. th. NGF Inj. | Michael G C et |
| immune reactivity | | | al. (1997) |
| | | | J Neurosci 17: |
| | | | 8476-90 |

(c) Receptors

| | | | |
|---|---|---|---|
| μ κ δ binding | SC-DH | monoarthritis | Besse D et al. |
| | | | (1992) |
| | | | Eur J |
| | | | Pharmacol 223: |
| | | | 123-31 |
| μ opiate receptors - | DRG | Carageenan ind. | Ji R-R et al. |
| immune reactivity | | inflammation | (1995) |
| | | | J Neurosci 15: |
| | | | 8156-66 |
| κ & δ opiate | DRG | Carageenan ind. | Ji R-R et al. |
| receptors —mRNA | | inflammation | (1995) |
| | | | J Neurosci 15: |
| | | | 8156-66 |
| κ & μ opiate | SC-DH | monoarthritis | Maekawa K et |
| receptors - mRNA | | | al. (1995) Pain |
| | | | 64: 365-71 |
| CCK_{B} receptors | DRG | Axotomy | Zhang X et al. |
| mRNA | | | (1993) |
| | | | Neuroscience |
| | | | 57: 227-233 |
| NMDA-R1-mRNA | SC-DH | CFA induced | Kus L et al. |
| | Laminae I & | inflammation | (1995) |
| | II | | Neuroscience |
| | | | 68: 159-65 |

(d) Enzymes

| | | | |
|---|---|---|---|
| NADPH diaphorase | SC-DH | Sciatic | Fiallos-Estrada |
| activity | | transection | et al. ('93) |
| | | | Neurosci Lett |
| | | | 150: 169-73 |
| NADPH diaphorase | SC | Formalin | Solodkin et al. |
| | | | 1992 |
| | | | Neurosci 51: |
| | | | 495-99 |
| NO synthetase | DRG | Axotomy | Verge V M K et |
| mRNA | | | al. (1992) |
| | | | PNAS 89: |
| | | | 11617-62 |
| NO synthetase | SC-DH | Formalin | Herdegen et al. |
| protein | | | (1994) |
| | | | Mol Brain Res |
| | | | 22: 245-58 |
| NO synthetase | DRG | Sciatic | Fiallos-Estrada |
| immune reactivity | | transection | et al. ('93) |
| | | | Neurosci Lett |
| | | | 150: 169-73 |

(e) Signal cascades

| | | | |
|---|---|---|---|
| rap1A, rap1B, H-ras | SC | Formalin | Urayama O et |
| mRNA | | | al. (1997) Mol |
| | | | Brain Res 45: |
| | | | 331-34 |
| PKC binding | SC-DH | CFA induced | Tölle T R et al |
| | | monoarthritis | (82) |
| | | | J Neurol 242 |
| | | | (S2): 135 |

(f) Transcription factors

| | | | |
|---|---|---|---|
| cFOS | SC | noxious | Hunt S P et al. |
| | | stimulation | (1987) |
| | | | Nature 328: |
| | | | 632-34 |
| cJun, JunB, cFOS | SC-DH | Formalin | Herdegen T et |
| Krox24 | | | al. (1994) Mol |
| | | | Brain Res 22: |
| | | | 245-48 |

SC, Spinal chord; DRG, dorsal root ganglia; CFA Complete Freund Adjunvans; NGF, Nerve Growth Factor

Hence the primary object of the invention was to develop a screening method for the identification of pain-relevant, in particular pain-regulating substances. The invention relates, therefore, to a method for discovering pain-regulating substances with the following process steps:

(a) incubation of a substance to be tested under appropriate conditions with a cell and/or a preparation from such a cell, which has synthesised a peptide or protein selected from the following group:

JNK3, PIM-2, LR-11, TFIIFβ, GGT-β, GATA3, CLC-7, catalase, tetraspanin (TM4SF/TSPAN-6/TM4-D), casein kinase 1a, MAP3K7/TAK-1 (TGF-β activated kinase), BAB14112 and/or spermidine synthase,

a peptide or protein which, or a part of which, is encoded by a polynucleotide according to any of FIGS. 34a), 34c), 34e), 34g), 35a), 35c), 35e), 36a), 36c), 37a), 37c), 38a), 38c), 39a), 39c), 39e), 40a), 40c), 40e), 41a), 41c), 41e), 42a), 42e), 43c), 43e), 44e), 45a), 45c), 45e), or 46e), or by a polynucleotide which is at least 90% similar thereto,

a peptide or protein having an amino acid sequence according to any of FIGS. 34b), 34d), 34f), 34h), 35b), 35d), 35f), 36b), 36d), 37b), 37d), 38b), 38d), 39b), 39d), 40b), 40d), 40f), 41b), 41d), 41f), 42b), 42f), 43d), 43f), 44f), 45b), 45d), 45f) or 46f), or a peptide or protein at least 90% similar thereto

and/or a protein which is encoded by a nucleic acid which, under stringent conditions, binds to a polynucleotide according to any of FIGS. 34a), 34c), 34e), 34g), 35a), 35c), 35e), 36a), 36c), 37a), 37c), 38a), 38c), 39a), 39c), 39e), 40a), 40c), 40e), 41a), 41c), 41e), 42a), 42e), 43c), 43e), 44e), 45a), 45c), 45e), or 46e), or to the antisense polynucleotide thereof,

a partial protein at least 10 amino acids long of any of the aforementioned proteins and/or peptides and/or

a peptide or protein encoded by a gene consisting of a polynucleotide, the nucleotide sequence whereof encompasses a gene fragment according to one of the sequences represented in FIGS. 1-33, or encoded by a polynucleotide at least 90% similar to such a gene,

(b) measurement of the binding of the test substance to the peptide or protein synthesised by the cell or measurement of at least one functional parameter altered by the binding of the test substance to the peptide or protein.

This novel screening method is based on being able to discover potential pain effectiveness of a substance by means of its interaction with a pain-regulated peptide or protein structure.

Here the term pain-regulating relates to a potential regulating influence on the physiological occurrence of pain, in particular to an analgesic effect. The term substance includes any compound suitable as an active ingredient in medicaments, in particular, therefore, low-molecular active ingredients, but also others such as nucleic acids, fats, sugars, peptides or proteins such as antibodies.

Incubation under appropriate conditions is understood here to mean that the substance to be examined can react with the cell or the corresponding preparation in an aqueous medium for a defined time before measurement. Here the aqueous medium can be at a temperature, for example, between 4° C. and 40° C., preferably at room temperature or at 37° C. The incubation time can be varied between a few seconds and a plurality of hours, depending on the interaction of the substance with the peptide or protein. Times between 1 min and 60 min are, however, preferred. The aqueous medium can contain appropriate salts and/or buffer systems, so that during incubation a pH of, for example between 6 and 8, preferably pH 7.0-7.5 prevails in the medium. Further appropriate substances such as coenzymes, nutrients etc. can be added to the medium. The appropriate conditions can be easily determined by a person skilled in the art on the basis of his experience, the literature or a few, simple preliminary experiments depending on the interaction to be examined of the substance with the peptide or protein, in order to obtain the clearest possible measurement in the method.

A cell which has synthesised a certain peptide or protein is a cell which has already endogenously expressed said peptide or protein or a cell which has been genetically altered so that it expresses said peptide or protein, and correspondingly contains said peptide or protein prior to the beginning of the method according to the invention. The cells can be cells either from immortalised cell lines or native cells originating from tissues and isolated therefrom, in which case the cells are no longer connected. The preparation from these cells encompasses, in particular, homogenates from the cells, the cytosol, a membrane fraction of the cells having membrane fragments, a suspension of isolated cell organelles etc.

In the scope of the present invention, the proteins and peptides listed here were identified as being regulated by pain by triggering pain in an animal and, after an appropriate time, comparing the expression pattern in certain tissues of the animal to those of a control animal in which pain had not been triggered. The differently expressed peptides and proteins found thereby also encompass known proteins such as JNK3, a protein kinase, PIM-2, a signalling kinase, LR-11, a mosaic protein from the family of low density lipoprotein receptors, TF11Fβ, a transcription factor, GGT-β, a subtype of geranylgeranyl transferase, GATA3, a transcription factor, CLC-7, a chloride channel, catalase, an enzyme of the oxygen detoxification pathway. In a further, more in depth analysis of the gene fragments discovered it has further been found that tetraspanin (TM4SF/TSPAN-6/TM4-D), which can be found specifically neuronally in the CNS, in particular inter alia in the cortex and hippocampus, and which is believed to play a role in the growth of neurites and the formation of synapses and to be a mediator between cell adhesion (integrins) and signal molecules (PKC, P14-Kinase), casein kinase 1a, which possibly contributes to the formation of synapses by means of phosphorylation of the AP-3 complex, MAP3K7/TAK-1 (TGF-β activated kinase), which specifically activates JNK kinases, BAB114112, a hitherto largely unknown protein, and spermidine synthase, responsible for growth, are also regulated by pain. For the functioning of the method it is not important from which species said proteins originate, but it is preferred if human, mouse or rat variants are used. The proteins named are known with regard to the DNA and amino acid coding sequence, and how they function in general has also been described. In the prior art, however they have not hitherto been linked to pain and in particular to the regulation of pain. Since here the identification of the proteins took place by means of a change in expression in an in-vivo pain model, the screening method according to the invention derived therefrom is significantly advantageous for future medicaments using said proteins, in that it builds not only on theoretical observations, but can also be presumed to possess strong in-vivo relevance. Since said method facilitates the interaction of substances with proteins and peptides not hitherto used in the field of pain as a criterion for the discovery of pain-regulating substances, it may now be possible, using said method, to discover pain-relevant substances which would have remained unnoticed in the methods known hitherto in the prior art using different peptides and proteins. This also is a significant advantage of the novel method according to the invention.

The proteins or peptides used can also be selected from those encoded by a polynucleotide according to one of FIGS. 34a), 34c), 34e), 34g), 35a), 35c), 35e), 36a), 36c), 37a), 37c), 38a), 38c), 39a), 39c), 39e), 40a), 40c), 40e), 41a), 41c), 41e), 42a), 42e), 43c), 43e), 44e), 45a), 45c), 45e), or 46e). Shown are most of the known mouse, rat and human cDNA coding sequences of JNK3, PIM-2, LR-11, TFIIFβ, GGT-β, GATA3, CLC-7 and catalase,as well as tetraspanin (TM4SF/TSPAN-6/TM4-D), casein kinase 1a, MAP3K7/TAK-1 (TGF-β activated kinase), BAB14112 and/or spermidine synthase, and in the case of rat GATA3 only a partial sequence and in the case of CCL-7, mouse genomic DNA is shown. Hitherto unknown in the prior art is the cDNA sequence of PIM-2, rat, which was cloned in the scope of the present invention. For the method according to the invention also peptides and proteins encoded by only one segment (part) of the polynucleotide can be used, although at least one polypeptide (>10 amino acids) or one protein must be used. Finally, under certain circumstances, also only partial segments of one of the aforenamed proteins of at least 10 amino acids are necessary for a screening method. Peptides and proteins encoded by a DNA sequence at least 90% similar to one of those shown can also be used, since such minor deviations usually do not influence the interaction with peptide and protein and therefore do not influence the functioning of the method. By 90% similarity a 90% correspondence in the sequence of bases in the coding region of the polynucleotide is understood.

The proteins can also be selected from those having an amino acid sequence according to any of FIGS. 34b), 34d), 34f), 34h), 35b), 35d), 35f), 36b), 36d), 37b), 37d), 38b), 38d), 39b), 39d), 40b), 40d), 40f), 41b), 41d), 41f), 42b), 42f), 43d), 43f), 44f), 45b), 45d), 45f) or 46f). These also are predominantly sequences known in the prior art, except for the sequence of PIM-2 rat, which was obtained in the scope of the present invention. Peptides and proteins having an amino acid sequence at least 90% similar to one of those shown can be used here, since minor deviations in the amino acid sequence also do not usually influence the interaction of the substance with peptide and protein and thus do not influence the functioning of the method. By 90% similarity a 90% correspondence in the sequence of amino acids is understood.

The proteins can also be selected from those encoded by a nucleic acid which, under stringent conditions, bind to a polynucleotide according to any of FIGS. 34a), 34c), 34e), 34g), 35a), 35c), 35e), 36a), 36c), 37a), 37c), 38a), 38c), 39a), 39c), 40a), 40c), 40e), 41a), 41c), 41e), 42a), 42e), 43c), 43e), 44e), 45a), 45c), 45e) or 46e) or to the antisense polynucleotide thereof Stringent conditions are understood here to mean conditions under which only perfectly base-paired nucleic acid strands are formed and remain stable, and antisense polynucleotide is understood to mean a molecule consisting of a plurality of natural or modified nucleic acids, the base sequence of which is complementary to the base sequence of a partial region of an RNA occurring in nature.

It may, in principle, be sufficient for the method according to the invention to use a partial protein at least 10 amino acids long of one of the aforementioned proteins and/or peptides, since 10 amino acids, preferably 15, in particular 20 amino acids are or can be fully specific.

The peptides and proteins can, however, also be selected from such compounds, encoded by a gene consisting of a polynucleotide, the nucleotide sequence of which encompasses a gene fragment according to any of the sequences represented in FIGS. 1-33. In the scope of the present invention pain-regulated gene fragments which have not been assigned to any known peptide or protein in the prior art were identified and sequenced. A postanalysis of the gene fragment, however, allowed assignment in some cases to tetraspanin (TM4SF/TSPAN-6/TM4-D), casein kinase 1a, MAP3K7/TAK-1 (TGF-β activated kinase), BAB14112 and/or spermidine synthase. In each case the sufficiently complete sequenced gene fragment clearly defines the relevant gene or polynucleotide, the sequence of which the gene fragment is a part. Because the corresponding gene fragment has been identified as pain-regulated a clearly physiological function of the gene is outlined. The person skilled in the art can attain the complete gene which encompasses the fragment using known methods. Thus a polynucleotide according to any of FIGS. 1-33 can be labelled as a probe, a cDNA bank can be hybridised with the probe and washed under stringent conditions and the cDNA clone to which the probe has bound can be isolated and, optionally, sequenced. Thus, the gene or polynucleotide can be obtained by obtaining and synthesising segments of a polynucleotide according to any of FIGS. 1-33 as gene-specific oligonucleotide primers, with which the extended polynucleotide is then generated and optionally sequenced by means of PCR, using single or double-stranded DNA, cDNA libraries or genomic DNA as a template. The procedure is explained more precisely later with an example. For this group of peptides and proteins also the advantage over the known method applies in that, when used in the method according to the invention, in-vivo relevance is assured and the method allows potentially pain-regulating substances to be identified, which in the screening methods hitherto known in the prior art might possibly have remained unnoticed.

The criterion by which the method allows the discovery of interesting substances is either the binding to the protein or peptide, which, e.g., can be shown by suppression of a known ligand or the amount of bound substance, or the alteration of a functional parameter by means of the interaction of the substance with the peptide or protein. Said interaction can, in particular, consist of regulation, inhibition and/or activation of receptors, ion channels and/or enzymes, and changed functional parameters can, for example, be the gene expression, the ion concentration, the pH or the membrane potential and the change in enzyme activity or concentration of the 2^{nd} messenger.

For explanation of the invention, as well as the explanations for terms in the main body of the text, further definitions are given below in order to clarify how certain terms, in particular those used in the claims, are to be understood and interpreted in the context of the present invention.

substance: a chemical compound is meant by this. Here compounds in the narrower sense are meant which can potentially bring about an effect in the body, low-molecular active ingredients, nucleic acids, fats, sugars, peptides or proteins, in particular here low-molecular active ingredients.

pain-regulating: in the context of the invention pain-regulating means that the substance directly or indirectly influences the perception of pain, in particular, of course, having an analgesic effect.

incubation: by incubation the introduction and leaving of a biological object of investigation is to be understood, for example a cell or a protein, at a certain temperature in a medium such as in an incubator or on a water-bath. Under appropriate conditions means here incubation under physiological conditions (e.g. 37° C. pH 7.2) or under the conditions which make optimal measurement possible in the method.

cell: the cell is a self-regulating, open system in flow equilibrium with its surroundings by means of permanent exchange of substances, having its own metabolism and ability to reproduce. The cell can be cultured separately or be part of a tissue, in particular of an organ, and may be present there either individually or also in association with other cells.

preparation from a cell: by this preparations are understood, which can be produced by means of chemical, biological, mechanical or physical methods by changing the cell structure, for example membrane fragments, isolated cell compartments, isolated cytosol, or homogenate obtained from tissue.

peptide: compound of amino acids linked in chains by peptide bonds. An oligopeptide consists of between 2 and 9 amino acids, a polypeptide consists of between 10 and 100 amino acids.

protein: compound of more than 100 amino acids linked in chains by peptide bonds, possibly having a defined spacial structure.

JNK3: protein kinase from the family of mitogen-activated protein kinases.

PIM-2: a proto-oncogene and a serine threonine kinase.

LR-11: a member of the LDL (low density lipoprotein) receptor family and a mosaic protein.

TFIIFβ: beta subunit of a transcription initiation factor, which binds to the RNA polymerase II.

GGT-β: beta subunit of geranyl geranyl transferase type 1 (EC 2.5.1.3.).

GATA3: type 3 of a GATA binding protein.

CLC-7: a chloride channel protein

catalase: an enzyme (EC 1.11.1.6) which plays a crucial role in the detoxification of reactive oxygen species.

tetraspanin (TM4SF/TSPAN-6/TM4-D): specifically found neuronally in the CNS, in particular inter alia in the cortex and the hippocampus. Probably plays a role in the growth of neurites and formation of synapses and seems to be a mediator between cell adhesion (integrins) and signal molecules (PKC, P14-kinase)

casein kinase 1a: ubiquitous enzyme, which possibly contributes to the formation of synapses by means of phosphorylation of the AP-3 complex.

MAP3K7/TAK-1 (TGF-β activated kinase): enzyme which specifically activates JNK kinases.

BAB14112: a hitherto largely unknown protein; AN: BAB14112,

spermidine synthase: ubiquitous enzyme, which seems to be responsible inter alia for cell growth.

polynucleotide: the basic nucleotide is a component of nucleic acids fundamentally consisting of nucleic base, pentose and phosphoric acid. Said nucleotide corresponds to a high-molecular polynucleotide of a plurality of nucleotides linked with one another via phosphoric acid pentose esterification. The present invention, however, also includes modified polynucleotides, which retain the base sequence, but possess a modified backbone instead of the phosphoric acid pentose.

at least 90 (95, 97)% similar: by this it is understood that the polynucleotides denoted are at least 90% (95%, 97%) identical to the reference (figure etc.) with regard to the base sequence in their coding region, the peptides and proteins denoted are at least 90% (95%, 97%) identical in their primary structure, and the amino acid sequences at least 90% (95%, 97%) identical to the reference.

gene: by the term gene a genome segment with a defined nucleotide sequence is referred to, which contains the information for synthesis of an mRNA, pre-mRNA or another RNA (e.g. tRNA, rRNA, snRNA etc.) It consists of coding and non-coding segments.

gene fragment: nucleic acid segment, which contains a part of a gene in its base sequence.

physiologically extended gene fragment: a gene fragment which is extended by means of molecular biological methods, such as e.g. the screening of a cDNA library, the extraction of complementary DNA strands from a nucleic acid mixture or methods using PCR (so-called RACE protocols) in such a way that it corresponds in its sequence to the mRNA expressed in the corresponding target organ (brain, spinal cord, dorsal root ganglion).

binding to the peptide or protein: interaction between substance and peptide or protein, which leads to fixation.

functional parameters: by this units of measurement of an experiment are meant, which correlate with the function of a protein (ion channel, receptor, enzyme).

genetically manipulated: manipulation of cells, tissues or organisms in such a way that genetic material is introduced.

endogenously expressed: expression of a protein, which, under appropriate culture conditions, shows a cell line without expression of said corresponding protein being induced by genetic manipulation

G protein: usual international abbreviation for a guanoseintriphosphate (GTP) binding protein which is activated as a signal protein by G protein coupled receptors.

reporter gene: general term for genes, the gene products whereof can be identified by means of simple biochemical methods or histochemical methods, such as, for example, luciferase, alkaline phosphatase or green fluorescent protein (GFP).

(recombinant) DNA construct: general term for any type of DNA molecules, which have originated by in vitro linking of DNA molecules.

cloning vector: general term for nucleic acid molecules, which, in cloning, serve as carriers of foreign genes or parts of said genes.

expression vector: term for specially constructed cloning vectors, which, after being introduced into an appropriate host cell, allow transcription and translation of the foreign gene cloned into the vector.

LTR sequence: abbreviation for long terminal repeat. General term for long sequence regions found at both ends of a linear genome. Such sequence regions occur, for example, in the genomes of retroviruses and at the ends of eukaryotic transposons.

poly A tail: the adenyl residues (ca.20-250) attached by polyadenylation at the 3′ end of messenger RNAs.

promoter sequence: term for a DNA sequence region from which the transcription of a gene i.e. the synthesis of mRNA is controlled.

ORI sequence: Abbreviation for origin of replication. The ORI sequence allows a DNA molecule to reproduce as an autonomous unit in the cell.

enhancer sequence: term for relatively short genetic elements, partly appearing as repetitions, which usually strengthen the expression of some genes to a variable extent.

transcription factor: term for a protein which influences the transciption of a gene by means of binding to specific DNA sequences.

culture: to keep cells or tissues under appropriate culture conditions.

conditions which allow expression: by this is understood the selection and use of culture conditions which allow expression of the protein in question, including temperature change, change of medium, addition of inducing substances, and leaving out of inhibiting substances,

incubation time: length of time for which cells or tissues are incubated, i.e. exposed to a defined temperature.

selection pressure: use of culture conditions which enhance growth in cells using a certain gene product known as the selection marker.

amphibian cell: cell from an animal belonging to the class amphibia.

bacteria cell: cell which is classified under the broad order of eubacteria or archaebacteria, or derives therefrom.

yeast cell: cell which is classified under the order of endomycetales, or derives therefrom.

insect cell: cell which is allocated to the class Hexapoda, or derives therefrom.

native mammal cell: cell originating from a mammal corresponding in its relevant characteristics to the cell found in the organism.

immortalised mammal cell: cell which, as a result of the culture conditions used or genetic manipulation, has acquired the characteristic of dividing more frequently than is usual (ca. 100) in the culture.

labelled: rendered accessible for a positive reaction by means of appropriate modification or derivatisation. For example by radioactivity, fluorescence or luminescence.

ligand: substance which binds to a molecule found in the body or in a cell, in particular a receptor.

suppression: complete or partial removal of a ligand from its binding point.

bound activity: biochemically or physically determined measurement which correlates to the amount of ligands bound to a receptor.

regulation: the inhibition or activation of a process resulting as part of a regulation process

inhibition: the prevention/lessening of a process as a special case in regulation

activation: the increasing of a process as a special case in regulation

receptors: in the broadest sense, all molecules present in the prokaryote or eukaryote organism which can bind to an active substance. In the narrower sense, membrane-bound proteins or complexes of a plurality of proteins, which bring about an alteration in the cell through binding of an active substance.

ion channels: membrane-bound proteins or complexes of a plurality of proteins via which cations or anions can penetrate the membrane.

enzymes: term for proteins or complexes of an activating non-protein component with a protein which possesses catalytic properties.

gene expression (express/expressible): the translation of the genetic information of a gene into RNA (RNA expression) or into protein (protein expression).

ion concentration: ion concentration of one or more ions in a particular compartment.

membrane potential: tension difference over a membrane because of a surplus of cations on the one side and anions on the other side of the membrane.

Change in enzyme activity: inhibition or induction of the catalytic activity of an enzyme.

2nd messenger: small molecule, which is formed in response to an extra cellular signal either in the cytosol, or which migrates into the cytosol and thus helps to pass on the information to the cell line, such as, for example, cAMP, IP₃.

(gene) probe: term for any type of nucleic acid, with the aid of which a gene which is sought or a certain DNA sequence can be detected. By means of derivatisation of the gene probe (e.g. biotin, magnetic beads, digoxin) DNA molecules can, additionally, be extracted from a mixture. Cloned genes, gene fragments, chemically synthesised oligonucleotides and also RNA, which is usually radioactively labelled, are used as probes

DNA: international term for deoxyribonucleic acid

genomic DNA: general term for the DNA originating from the nucleus of a cell in eukaryotic organisms.

cDNA: abbreviation for complementary DNA. Term for the single or double stranded DNA copy of an RNA molecule.

cDNA bank/library: term for a collection of arbitrarily cloned cDNA fragments, which, taken together, represent the entire RNA synthesised by a cell or a tissue.

cDNA clone: term for a population of genetically identical cells, which are derived from a single cell such that said cell contains an artificially introduced cDNA fragment.

hybridisation: formation, by means of base pairing, of a double-stranded nucleic acid molecule from two separate single strands.

a stringent conditions: conditions under which only perfectly base-paired nucleic acid strands are formed and remain stable.

isolate: to detect and separate a desired molecule from a mixture.

DNA sequencing: determination of the sequence of bases in a DNA molecule.

nucleic acid sequence: term for the primary structure of a DNA molecule, i.e. the sequence of the individual bases from which DNA is composed.

gene-specific oligonucleotide primers: oligonucleic acids, i.e. nucleic acid fragments 10-40 bases long, which, in their base composition, allow stringent hybridisation to the desired gene or the desired cDNA

determination of oligonucleotide primers: the manual or computer-aided search for oligonucleotides belonging to a given DNA sequence which are optimally suited for a hybridisation and/or a polymerase chain reaction.

PCR: abbreviation for polymerase chain reaction. In vitro method for selective enrichment of nucleic acid regions of defined length and defined sequence from a mixture of nucleic acid molecules.

DNA template: nucleic acid molecule or a mixture of nucleic acid molecules from which a DNA segment is amplified using PCR (see above).

RNA: usual international abbreviation for ribonucleic acids.

mRNA: usual international abbreviation for messenger ribonucleic acids involved in the transfer of genetic information from the nucleus into the cell and which contain information for the synthesis of a polypeptide or a protein.

antisense polynucleotide: a molecule, consisting of a plurality of natural or modified nucleic acids, whose base sequence is complementary to the base sequence of a partial region of an RNA occuring in nature.

PNA: usual international abbreviation for peptidic nucleic acids. Here amino acids linked by peptide bonds form a chain and the amino acids carry a base, capable of hybridising with DNA or RNA, as a side chain.

sequence: sequence of nucleotides or amino acids. In the specific context of the present invention the nucleic acid sequence is meant.

ribozyme: term for an RNA with catalytic capacity (e.g. ligase, endonuclease, polymerase, exonuclease).

DNA enzyme: term for a DNA molecule containing catalytic activity (e.g. ligase, endonuclease, polymerase, exonuclease).

catalytic RNA/DNA: general term for ribozymes and DNA enzymes (see above).

adenovirus: cytopathogenic virus occurring in vertebrates.

adeno-associated virus (AAV): belongs to the parvovirus family. For effective multiplication of AAV a coinfection of the host cells with auxiliary viruses (e. g. herpes, vaccinia or adenoviruses) is needed. The characteristic of AAV of stable integration into the host genome makes it particularly interesting as a transduction vector for mammalian cells.

herpes virus: viral cause of herpes infection.

post-translational modification: alteration in proteins or polypeptides carried out after translation, including, e.g phosphorylation, glycosylation, amidation, acetylation or proteolysis.

glycosylate: term for attaching single sugar molecules or whole chains of sugars to proteins.

phosphorylate: term for attaching one or more phosphate residues to a protein, preferably to the OH groups of amino acids serine, threonine or tyrosine.

amidate: term for the transformation of a carboxyl function into an amide function, e.g. to the carboxyterminal amino acid residue of a peptide or protein.

provide with a membrane anchor: modification, after translation, of a protein or other organic molecule in such a way that it is anchored by attaching a hydrophobic molecule, appropriately a fatty acid or derivative thereof, to the double layer lipid membrane of cells.

cleave: in the current specific case, the cleavage of a peptide or protein into a plurality of subsequences.

reduce: to reduce a molecule consisting of a plurality of individual parts by one or more parts.

antibodies: proteins called immunoglobins which are either soluble or bound to cell membranes having a specific combining site for antigens.

monoclonal antibodies: antibodies having extremely high selectivity for a single antigenic determinant of an antigen.

polyclonal antibodies: mixture of antibodies for a plurality of determinants of an antigen.

transgenic: genetically altered

non-human mammal: any mammal (Mammalia class) with the exception of the human species.

germ cell: cell having a haploid genome, which by means of fusion with a second germ cell makes the formation of a new organism possible.

somatic cell: diploid cell as component of an organism.

introduction of one or more chromosomes: intervention in the nucleotide sequence on a chromosomatic level.

genome: general term for the totality of all genes in an organism.

ancestor of the animal: an animal (the ancestor), which is directly related by natural or artificial means to another animal (the descendant) by transmission of its genetic material.

expressible: a nucleic acid molecule is expressible if it contains information for synthesis of a protein or polypeptide and is equipped with appropriate regulatory sequences allowing synthesis of said protein or polypeptide either in vitro or in vivo. If these prerequisites are no longer fulfilled, for example on account of intervention in the coding sequence, the nucleic acid molecule is no longer expressible.

rodent: animal of the order Rodentia, e.g. rat or mouse.

substance identifiable as pain-regulating: substance which, when introduced into a living organism, brings about a change in behaviour described by the person skilled in the art as pain-inhibiting (anti-nociceptive, anti-hyperalgesic or anti-allodynic). In the case of the screening process, this means that during the screening the substance clearly exceeds, for example by 100%, the binding or interaction of the average of the tested substances by stronger binding or producing a change in a functional parameter.

compound: another name for molecule, consisting of a plurality of atoms, here a molecule identified by the method according to the invention.

active ingredient: a compound which, when used on an organism, brings about a change in said organism. In particular, organic-chemically synthesised molecules are meant, which exert a healing effect on the organism. Here, in particular, molecules which bind to the proteins and peptides according to the invention.

low-molecular: molecule with a molecular weight <2 kDa.

medicament: a substance according to the definition in article 1 §2 of the law concerning the trade in medicaments.

diagnostic reagent: compound or method which can be used in order to diagnose an illness.

treatment of pain: method with the aim of reducing or relieving pain, or inhibiting the expected emergence of pain (pre-emptive analgesia).

chronic pain: a long-lasting sensation of pain, often characterised in that it persists beyond the time and place of the initial stimulus and the pain sensitivity of the body increases.

gene therapy: by gene therapy all methods are understood which are aimed at treating the cause of genetic illnesses by means of appropriate changes in the genome.

in vivo gene therapy: introduction of genetic material into the living organism for the purpose of gene therapy. Intervention via somatic and germ cells differs in that the one is carried out on diploid cells and the other on haploid cells.

in vitro gene therapy: introduction of genetic material into cells outside the human body with the aim of using said cells again later by introducing them into the human body for the purpose of gene therapy.

diagnosis: method for identifying an illness.

study of effectiveness: study with the aim of investigating the effectiveness of a compound for its influence on a living organism.

In a preferred embodiment of the method the cell is genetically manipulated before step (a). Here genetic material is introduced into the cell, in particular one or more polynucleotide sequences. In a farther preferred variation of said embodiment genetic manipulation allows the measurement of at least one of the functional parameters changed by the test substance. In said embodiment conditions are created by means of genetic manipulation under which the alteration of a functional parameter can be measured, or under which the alteration can be better measured. It is especially preferred if a form of a G-Protein not endogenously expressed in the cell is expressed by the genetic manipulation, or if a reporter gene is introduced. Here, in particular, the genetic introduction into the cell of a G protein not endogenically available or not physiologically expressed (a GTP binding protein) is meant, for example, the introduction of a chimeric G protein which allows a change in the signal path or of a promiscuous G protein which binds very easily. The introduction of a reporter gene, on the other hand, allows measurement of induced expression of the gene product (i.e. expression triggered outside the cell).

In a further preferred embodiment the cell is genetically manipulated in such a way that the cell contains at least one polynucleotide according to any of FIGS. 34a), 34c), 34e), 34g), 35a), 35c), 35e), 36a), 36c), 37a), 37c), 38a), 38c), 39a), 39c), 39e), 40a), 40c), 40e), 41a), 41c), 41e), 42a), 42e), 43c), 43e), 44e), 45a), 45c) 45e), or 46e) or a polynucleotide at least 90% similar or a polynucleotide of the coding sequence of a gene which contains a gene fragment according to any of the sequences represented in FIGS. 1-33, or a polynucleotide at least 90% similar thereto. Thus it is possible, for example, for a peptide or protein which is not endogenously expressed in the cell or preparation used in the method to be synthesised by the cell. Here it is particularly preferred if the polynucleotide is contained in a recombinant DNA construct. By a (recombinant) DNA construct a DNA molecule produced in vitro is understood.

If, in the method, the cell is genetically manipulated before step (a), it us preferred that, after the genetic manipulation and before step (a), the cell is cultivated under conditions which allow expression, optionally under selection pressure. Cultivation is understood as the keeping of cells or tissues under conditions which ensure survival of the cells and their succeeding generations. Here the conditions should be selected in such a way as to allow expression of the material added by the genetic manipulation. For this purpose pH, oxygen content and temperature should be physiologically maintained and sufficient nutrients and necessary cofactors should be added. Selection pressure allows only the cells in which the genetic manipulation has been at least partly successful to be further cultivated. This includes, for example, the introduction of antibiotic resistance via the DNA construct.

In the method according to the invention it is particularly preferred if the cell used is an amphibian cell, a bacteria cell, a yeast cell, an insect cell or an immortalised or native mammalian cell. Examples of amphibian cells are Xenopus oocytes, examples of bacteria cells are E.coli cells, examples of yeast cells Saccharomyces cerevisiae, examples of insect cells Sf9 cells, examples of immortalised mammalian cells HeLa cells und examples of native mammalian cells are CHO (Chinese Hamster Ovary) cells

In a preferred measuring method for determining the binding of the substance to peptide or protein in the method according to the invention the measurement of the binding takes place by means of suppression a known labelled ligand of the peptide or protein and/or by means of the activity bound thereto of a labelled test substance. Here a ligand is a molecule binding highly specifically to the protein or peptide, which is suppressed from the attachment site by another binding substance to be tested. Labelling is understood as meaning an artificial modification in the molecule which allows it to be traced. Examples are radioactive, fluorescent or luminescent labelling.

In a further preferred measuring method to determine the change in the functional parameters triggered by the binding of the substance to peptide or protein in the method according to the invention, measurement of at least one of the functional parameters changed by the test substance takes places by means of measurement of the regulation, inhibition and/or activation of receptors, ion channels and/or enzymes, in particular by means of measurement of the change in gene expression, the ion concentration, the pH or the membrane potential, via change in the enzyme activity or the concentration of the 2nd messenger. Thus on the one hand the measurement of the effectiveness of the substance over the influence of receptors, ion channels, and/or enzymes is directly included and on the other hand examples preferably to be measured are changing parameters such as gene expression, ion milieu, pH, membrane potential, enzyme activity or concentration of the 2^{nd} messenger. By ion milieu in particular the concentration of one or more ions in a cell compartment is understood, in particular the cytosol. By membrane potential the loading difference between two sides of a biomembrane is understood and by 2^{nd} messenger the messenger material of the intracellular signal path such as e.g. cyclic AMP (cAMP), inosotoltriphosphate(IP3) or diacylglycerol (DAG)

In a preferred variation of the method the peptide or protein in steps (a) and (b) is selected from the following groups:

JNK3, PIM-2, LR-11, TFIIFβ, GGT-β, GATA3, CLC-7, catalase, tetraspanin (TM4SF/TSPAN-6/TM4-D), casein kinase 1a, MAP3K7/TAK-1 (TGF-β activated kinase), BAB14112 and/or spermidine synthase,

a peptide or protein which, or part of which, is encoded by a polynucleotide according to any of FIGS. 34a), 34c), 34e), 34g), 35a), 35c), 35e), 36a), 36c), 37a), 37c), 38a), 38c), 39a), 39c), 39e), 40a), 40c), 40e), 41a), 41c), 41e), 42a), 42e), 43c), 43e), 44e), 45a), 45c), 45e), or 46e), or by a polynucleotide which is at least 90% preferably 95%, in particular 97% similar thereto, or

a peptide or protein having an amino acid sequence according to any of FIGS. 34b), 34d), 34f), 34h), 35b), 35d), 35f), 36b), 36d), 37b), 37d), 38b), 38d), 39b), 39d), 40b), 40d), 40f), 41b), 41d), 41f), 42b), 42f), 43d), 43f), 44f), 45b), 45d), 45f) or 46f) or a peptide or protein at least 90%, preferably 95%, in particular 97% similar thereto

and/or a protein, which is encoded by a nucleic acid which, under stringent conditions, binds to a polynucleotide according to any of FIGS. 34a), 34c), 34e), 34g), 35a), 35c), 35e), 36a), 36c), 37a), 37c), 38a), 38c), 39a), 39c), 39e), 40a), 40c), 40e), 41a), 41c), 41e), 42a), 42e), 43c), 43e), 44e), 45a), 45c), 45e), or 46e) or to the antisense polynucleotide thereof and/or

a partial protein at least 10 preferably 15, in particular at least 20 amino acids long, of any of the aforementioned proteins and/or peptides.

Included here is the use of peptides and in particular proteins having a known sequence and function, but where no function relating to pain was known in the prior art for said peptides and proteins.

In a further preferred variation of the method the peptide or protein in steps (a) and (b) is encoded by a gene consisting of a polynucleotide containing a gene fragment according to any of the sequences represented in FIGS. 1, 3-8, 10, 11, 13, 14, 15, 17-27 or 31-33, or a polynucleotide at least 90%, in particular 95%, preferably 97% similar thereto.

In a further preferred variation of the method the peptide or protein in steps (a) and (b) is encoded by a gene consisting of a polynucleotide containing a gene fragment according to any of the sequences represented in FIGS. 2, 9, 12, 16 or 28-30, or a polynucleotide at least 90%, in particular 95%, preferably 97% similar thereto.

Preferably the invention further relates to a polynucleotide corresponding by at least 90%, preferably 95%, in particular at least 97% to any of the nuclear acid sequences represented in any of FIGS. 1-33 or to a polynucleotide from the coding sequence of a gene containing a gene fragment according to any of FIGS. 1-33: Even the gene fragments represented are included here, as is a polynucleotide corresponding either completely or at least to parts of the coding sequence of the gene corresponding to the fragment. Thus also polynucleotides which show at least 90%, preferably 95%, in particular 97% similarity in their base sequence to the coding sequence of the polynucleotides represented or the coding sequence of the gene are meant.

The first particularly favoured form of the polynucleotide is a polynucleotide corresponding by at least 90%, preferably 95%, in particular at least 97% to any of the nucleotide sequences represented in any of FIGS. 1, 3-8, 10, 11, 13, 14, 15, 17-27 or 31-33, in particular 20 or 21 or to parts thereof, or to a polynucleotide from the coding sequence of a gene containing a gene fragment according to any of FIGS. 1, 3-8, 10, 11, 13, 14, 15, 17-27 oder 31-33, in particular 20 or 21.

Preferably the invention further relates to a first polynucleotide defined by the current production method, namely a polynucleotide corresponding by at least 90%, preferably 95%, in particular by at least 97% to a defined nuclear acid sequence obtainable by labelling a polynucleotide according to any of FIGS. 1-33 as a probe, hybridising a cDNA bank with the probe and washing under stringent conditions and isolating and optionally sequencing the cDNA clone to which the probe has bound. Here a polynucleotide or gene, defined by the production method by which a certain product, the gene containing a certain known gene fragment, is isolated and can be sequenced. A probe is a nucleic acid which can be used for the identification of complementary or corresponding nucleotide sequences and which is usually labelled so that it can be identified. A cDNA bank is made up of cloned cDNA fragments of a cell or a tissue which should reproduce the mRNA of the chosen tissue as completely as possible. Hybridisation means the binding of two single-stranded nucleic acid molecules and washing under stringent conditions is designed to ensure that only nucleic acid molecules hybridised by exactly paired bases remain bound. A cDNA clone is a genetically identical group of descendants, here having identical cDNA.

Preferably the invention further relates to a second polynucleotide defined by the production method, corresponding by at least 90%, preferably 95%, in particular by at least 97% to a defined nuclear acid sequence obtainable by determining and synthesising segments of a polynucleotide according to any of FIGS. 1-33 as gene-specific oligonucleotide primers, with which the extended polynucleotide is then generated and, optionally, sequenced by means of PCR using single or double-stranded DNA from cDNA libraries or genomic DNA as a template. Here also a polynucleotide or gene is defined by means of a further production process by which a certain product, the gene containing a certain known gene fragment, is isolated and can be sequenced. Primer is an oligonucleotide, which hybridises with the target DNA (as a so-called template) and is the point of departure for the synthesis. PCR is the abbreviation for polymerase chain reaction, in which the heat resistance of a certain polymerase is used for selective amplification. Here the template serves as starting material on which the amplification takes place selectively with the primers of suitable hybridising DNA.

The second particularly favoured form of the polynucleotide is the form defined by the production method, in particular when a polynucleotide (gene fragment) according to any of FIGS. 1, 3-8, 10, 11, 13, 14, 15, 17-27 or 31-33, in particular 20 or 21, is used as the point of departure for production/isolation/sequencing.

It is further preferred if the polynucleotide is an RNA or single or double-stranded DNA, in particular mRNA or cDNA.

It is likewise preferred if the polynucleotide is an antisense polynucleotide or PNA showing a sequence capable of binding specifically to a polynucleotide according to the invention. By PNA, peptidic nucleic acid is understood, which carries the base pairs, but whose backbone is peptidically bound. An antisense polynucleotide shows the complementary base sequence to at least one part of a base nucleic acid. It is likewise preferred if the polynucleotide is part of a ribozyme or other DNA enzyme or of a catalytic RNA or DNA. By ribozyme a catalytically active ribonucleic acid is understood, by DNA enzyme a corresponding deoxyribonucleic acid, that is catalytic RNA or DNA.

The invention further relates to a vector containing any of the polynucleotides already described. By vector a nucleic acid molecule is understood which, in genetic manipulation, serves to contain or transmit foreign genes. It is especially preferred if it is an expression vector. It then promotes expression of the foreign gene or polynucleotide contained.

Further preferred is a vector derived from a virus, for example the adeno virus, adeno-associated virus or herpes virus and/or that it contains at least one LTR, poly A, promotor and/or ORI sequence. An LTR is a long terminal repeat, a segment found at the end, for example in viruses. A poly A sequence is a tail more than 20 adenosine residues long. A promotor sequence is the region which controls transcription.

A particularly favoured form of the vector according to the invention is a vector containing a polynucleotide or a polynucleotide at least 90%, preferably 95%, in particular 97% similar thereto, according to FIGS. 1, 3-8, 10, 11, 13, 14, 15, 17-27 or 31-33, in particular 20 or 21 or a polynucleotide or a polynucleotide at least 90%, preferably 95%, I particular 97% similar thereto containing the sequence according to FIGS. 1, 3, 8, 10, 11, 13, 14, 15, 17-27 or 31-33, in particular 20 or 21, or a polynucleotide or a polynucleotide at least 90%, preferably 95%, in particular 97% similar thereto, obtained by means of a production method using a gene fragment according to any of FIGS. 1, 3-8, 10, 11, 13, 14, 15, 17-27 or 31-33, in particular 20 or 21.

The invention further relates to a peptide, in particular an oligopeptide or polypeptide, or a protein encoded by any of the polynucleotides already described as subjects of the invention.

The invention further relates to a peptide, in particular an oligopeptide or polypeptide or a protein encoded by a polynucleotide which, under stringent conditions, hybridises with any of the polynucleotides according to FIGS. 1-33 or with any of the antisense polynucleotides thereof.

The invention also relates to whether the peptide or protein has been modified after translation, in particular by glycosylation, phosphorylation, amidation, methylation, acetylation, ADP ribosylisation, hydroxylation, being provided with a membrane anchor, cleaved or reduced. Modifications after translation can, for example, be taken from Voet/Voet, Biochemistry, 1st Edition, 1990 pp 935-938.

A particularly favoured form of the peptide or protein is obtained if it is encoded by a polynucleotide or a polynucleotide at least 90%, preferably 95%, in particular 97% similar thereto according to FIGS. 1, 3-8, 10, 11, 13, 14, 15, 17-27 or 31-33, in particular 20 or 21, or by a polynucleotide or encoded by a polynucleotide at least 90%, preferably 95%, in particular 97% similar thereto containing the sequence according to FIGS. 1, 3-8, 10, 11, 13, 14, 15, 17-27 or 31-33, in particular 20 or 21, or a polynucleotide, or a polynucleotide at least 90%, preferably 95%, in particular 97% similar thereto obtained by means of a production method using a gene fragment according to any of FIGS. 1, 3-8, 10, 11, 13, 14, 15, 17-27 or 31-33, in particular 20 or 21.

The invention further relates to antibodies for a peptide or protein already described as a subject of the invention. Here it is preferred if the antibody is a monoclonal or polyclonal antibody.

The particularly favoured form of the antibody is for a peptide or protein encoded by a polynucleotide according to FIGS. 1, 3-8, 10, 11, 13, 14, 15, 17-27 or 31-33, in particular 20 or 21 or a polynucleotide at least 90%, preferably 95%, in particular 97% similar thereto, or encoded by a polynucleotide, or a polynucleotide at least 90%, preferably 95%, in particular 97% similar thereto containing the sequence according to FIGS. 1, 3-8, 10, 11, 13, 14, 15, 17-27 or 31-33, in particular 20 or 21 or by a polynucleotide or a polynucleotide at least 90%, preferably 95%, in particular 97% similar thereto obtained by means of a production method using a gene fragment according to any of FIGS. 1, 3-8, 10, 11, 13, 14, 15, 17-27 or 31-33, in particular 20 or 21.

The invention further relates to a cell containing a polynucleotide already described as a subject of the application, a peptide or protein already described as a subject of the application and/or or a vector already described as a subject of the application. It is particularly preferred if the cell is an amphibian cell, a bacteria cell, a yeast cell, an insect cell or an immortalised or native mammalian cell. Examples of amphibian cells are Xenopus oocytes, examples of bacteria cells are E.coli cells, examples of yeast cells Saccharomyces cerevisiae, examples of insect cells Sf9 cells, examples of immortalised mammalian cells HeLa cells and of native mammalian cells CHO (Chinese Hamster Ovary) cells.

The particularly favoured form of the cell contains the particularly favoured form of the polynucleotide, the particularly favoured form of the peptide or protein and/or the particularly favoured form of the vector.

The invention further relates to a transgenic non-human mammal, whose germ cells and somatic cells contain a polynucleotide already described as a subject of the invention as the result of introducing a chromosome or chromosomes into the genome of the animal or into the genome of any of the predecessors of said animal. By introducing a chromosome or chromosomes it is understood that the genetic manipulative intervention produces an effect in the chromosome of the animal.

The invention further relates to a transgenic non-human mammal, whose germ cells and somatic cells contain any of the nucleotide sequences according to any of Claims 14-21 as the result of chromosomal manipulation in the genome of the animal or in the genome of any of the predecessors of said animal, the sequence no longer being in expressible form. The chromosomal manipulation related to the gene of the animal or its ancestors. By no longer expressible it is understood that the information for synthesis of a polypeptide or of a protein, although available in native form, no longer allows complete synthesis. Examples are alteration of the regulatory sequences or cutting out of a part of the native nucleic acid molecule in the coding region.

It is particularly preferred if the transgenic non-human mammal is a rodent.

The particularly favoured form of the transgenic non-human mammal is when the nucleotide sequence corresponds to the particularly favoured form of the polynucleotide.

The invention further relates to a compound identifiable as a pain-regulating substance by means of a method according to the invention. Here compound relates in particular to low-molecular active ingredients, but also to peptides, proteins and nucleic acids. Identifiable means here that, in the screening method according to the invention, the compound shows the characteristic of binding substantially more strongly, preferably twice as strongly as the average of the substances to be tested or, with respect to the change in functional parameters, it deviates clearly from the average of the substances to be tested.

A particularly favoured form of the compound according to the invention is identifiable as a pain-regulating substance by means of a method using the known proteins JNK3, PIM-2, LR-11, TFIIFβ, GGT-β, GATA3, CLC-7, catalase, tetraspanin (TM4SF/TSPAN-6/TM4-D), casein kinase 1a, MAP3K7/TAK-1 (TGF-β activated kinase), BAB14112 and/or spermidine synthase, a peptide or protein which or part of which is encoded by a polynucleotide according to any of FIGS. 34a), 34c), 34e), 34g), 35a), 35c), 35e), 36a), 36c), 37a), 37c), 38a), 38c), 39a), 39c), 39e), 40a), 40c), 40e), 41a), 41c), 41e), 42a), 42e), 43c), 43e), 44e), 45a), 45c), 45e), or 46e) or by a polynucleotide which is at least 90% preferably 95%, in particular 97% similar thereto, or a peptide or protein having an amino acid sequence according to any of FIGS. 34b), 34d), 34f), 34h), 35b), 35d), 35f), 36b), 36d), 37b), 37d), 38b), 38d), 39b), 39d), 40b), 40d), 40f), 41b), 41d), 41f), 42b), 42f), 43d), 43f), 44f), 45b), 45d), 45f) or 46f) or a peptide or protein at least 90%, preferably 95%, in particular 97% similar thereto and/or a protein, encoded by a nucleic acid which, under stringent conditions, binds to a polynucleotide according to any of FIGS. 34a), 34c), 34e), 34g), 35a), 35c), 35e), 36a), 36c), 37a), 37c), 38a), 38c), 39a), 39c), 39e), 40a), 40c), 40e), 41a), 41c), 41e), 42a), 42e), 43c), 43e), 44e), 45a), 45c), 45e), or 46e), or the antisense thereof and/or a partial protein at least 10, preferably 15, in particular at least 20 amino acids long, of one or the aforementioned proteins and/or peptides.

An equally particularly favoured form of the compound according to the invention is identifiable by means of a method in which the peptide or protein in steps (a) and (b) is encoded by a gene consisting of a polynucleotide containing a gene fragment according to any of FIGS. 1, 3-8, 10, 11, 13, 14, 15, 17-27 or 31-33, in particular 20 or 21, or a polynucleotide at least 90%, in particular 95%, preferably 97% similar to the gene.

Equally preferred is a compound identifiable by means of a method in which the peptide or protein in steps (a) and (b) is encoded by a gene consisting of a polynucleotide containing a gene fragment according to any of FIGS. 2, 9, 12, 16 or 28-30, or a polynucleotide at least 90%, in particular 95%, preferably 97% similar to the gene.

The invention further relates to an active ingredient which binds to a peptide or protein according to the invention, and it is particularly preferred if the active ingredient is a low-molecular active ingredient.

A particularly favoured form of the active ingredient is if the active ingredient binds to the particularly favoured form of the peptide or protein.

The invention further relates to a medicament containing at least one polynucleotide according to the invention, one peptide or protein according to the invention, one vector according to the invention, one antibody according to the invention, one cell according to the invention, one compound according to the invention and/or one active ingredient according to the invention and, optionally, appropriate auxiliary or additional substances. The medicaments according to the invention can be administered in liquid form, in the form of injection solutions, drops or mixtures, as semi-solid medicaments in the form of granules, tablets, pills, patches, capsules, plasters or aerosols and, depending on the form of the medicament, can optionally contain carrier material, thickening, solvents, diluents, colouring and binding agents as well as at least one subject according to the invention. The choice of auxiliary substances and the amounts of the same to be used depends on whether the medicament is to be applied orally, perorally, parenterally, intravenously, intraperitoneally, intradermally, intramuscularly, intranasally, buccally, rectally, or locally, for example on to infections on the skin, mucus membranes and on the eyes. For oral application preparations in the form of tablets, dragees, capsules, granules, drops, liquids and syrups are suitable, for parenteral or topical application and for inhalation solutions, suspensions, easily reconstitutable dry preparations and sprays are suitable. Subjects according to the invention in dissolved form in a depot or in a plaster, optionally with additives to promote skin penetration, are appropriate preparations for percutaneous application. Preparations which can be used orally or percutaneously can release the subjects according to the invention over a delayed period of time. The quantity of the active ingredient to be administered to patients varies depending on the weight of the patient, the method of application, the indication and the seriousness of the illness. Usually 2 to 500 mg/kg of at least one subject according to the invention are administered. If the medicament is to be used in particular for gene therapy, a physiological saline solution, stabilisers, proteinase inhibitors, DNAse inhibitors etc are recommended as appropriate auxiliary or additional substances.

Furthermore, a medicament containing preferred forms or the particularly favoured form(s) of the polynucleotide(s), peptide or protein, vector, antibody, cell, compound and/or active ingredient is particularly preferred.

Here it can be particularly preferred if the medicament contains a preferred compound according to the invention identifiable as a pain-regulating substance by means of a method using the known proteins JNK3, PIM-2, LR-11, TFIIFβ, GGT-β, GATA3, CLC-7, catalase, tetraspanin (TM4SF/TSPAN-6/TM4-D), casein kinase 1a, MAP3K7/TAK-1 (TGF-β activated kinase), BAB14112 and/or spermidine synthase, a peptide or protein which, or a part of which, is encoded by a polynucleotide according to any of FIGS. 34a), 34c), 34e), 34g), 35a), 35c), 35e), 36a), 36c), 37a), 37c), 38a), 38c), 39a), 39c), 39e), 40a), 40c), 40e), 41a), 41c), 41e), 42a), 42e), 43c), 43e), 44e), 45a), 45c), 45e), or 46e) or by a polynucleotide which is at least 90%, preferably 95%, in particular 97% similar thereto, or a peptide or protein having an amino acid sequence according to any of FIGS. 34b), 34d), 34f), 34h), 35b), 35d), 35f), 36b), 36d), 37b), 37d), 38b), 38d), 39b), 39d), 40b), 40d), 40f), 41b), 41d), 41f), 42b), 42f), 43d), 43f), 44f), 45b), 45d), 45f) or 46f) or a peptide or protein at least 90%, preferably 95%, in particular 97% similar thereto, and/or a protein encoded by a nucleic acid which, under stringent conditions, binds to a polynucleotide according to any of FIGS. 34a), 34c), 34e), 34g), 35a), 35c), 35e), 36a), 36c), 37a), 37c), 38a), 38c), 39a), 39c), 39e), 40a), 40c), 40e), 41a), 41c), 41e), 42a), 42e), 43c), 43e), 44e), 45a), 45c), 45e), or 46e) or the antisense thereof and/or a partial protein at least 10, preferably 15, in particular at least 20 amino acids long, of one of the aforementioned proteins and/or peptides.

It is also preferred if the medicament is a compound identifiable by means of a method in which the peptide or protein in steps (a) and (b) is encoded by a gene consisting of a polynucleotide containing a gene fragment according to any of FIGS. 1, 3-8, 10, 11, 13, 14, 15, 17-27 or 31-33, in particular 20 or 21, or by a polynucleotide at least 90%, in particular 95%, preferably 97% similar to the gene, or contains an active substance which binds to the particularly favoured form of the peptide or protein.

The invention further relates to a diagnostic reagent containing at least one polynucleotide, peptide or protein, vector, antibody or parts thereof according to the invention, and/or one cell according to the invention and, optionally appropriate additives. By diagnostic reagent an aid to diagnosis, for example of an occurrence of illness, is understood here.

A diagnostic reagent containing the particularly favoured form of the polynucleotide, peptide or protein or a part thereof, of the vector, antibody, and/or cell is particularly preferred.

Also preferred is a form of the diagnostic reagent containing a preferred polynucleotide, where this is an antisense polynucleotide or PNA showing a sequence capable of binding specifically to a polynucleotide according to the invention.

The invention further relates to the use according to the invention of a polynucleotide, peptide or protein, vector, antibody, cell, compound, active ingredient and/or an active ingredient which binds to a peptide or protein selected from the following group for the production of a medicament for the treatment of pain:

JNK3, PIM-2, LR-11, TFIIFβ, GGT-β, GATA3, CLC-7, catalase, tetraspanin (TM4SF/TSPAN-6/TM4-D), casein kinase 1a, MAP3K7/TAK-1 (TGF-β activated kinase), BAB14112 and/or spermidine synthase,

a peptide or protein, which or a part of which is encoded by a polynucleotide according to any of FIGS. 34a), 34c), 34e), 34g), 35a), 35c), 35e), 36a), 36c), 37a), 37c), 38a), 38c), 39a), 39c), 39e), 40a), 40c), 40e), 41a), 41c), 41e), 42a), 42e), 43c), 43e), 44e), 45a), 45c), 45e), or 46e) or by a polynucleotide which is at least 90% preferably 95%, in particular 97% similar thereto

a peptide or protein having an amino acid sequence according to any of FIGS. 34b), 34d), 34f), 34h), 35b), 35d), 35f), 36b), 36d), 37b), 37d), 38b), 38d), 39b), 39d), 40b), 40d), 40f), 41b), 41d), 41f), 42b), 42f), 43d), 43f), 44f), 45b), 45d), 45f) or 46f) or a peptide or protein at least 90%, preferably 95%, in particular 97% similar thereto

and/or a protein, which is encoded by a nucleic acid which, under stringent conditions, binds to a polynucleotide or the antisense thereof according to any of FIGS. 34a), 34c), 34e), 34g), 35a), 35c), 35e), 36a), 36c), 37a), 37c), 38a), 38c), 39a), 39c), 39e), 40a), 40c), 40e), 41a), 41c), 41e), 42a), 42e), 43c), 43e), 44e), 45a), 45c), 45e), or 46e) and/or

a partial protein at least 10 preferably 15, in particular at least 20 amino acids long, of any of the aforementioned proteins and/or peptides.

Particularly preferred is the use for the treatment of chronic pain.

Also preferred is use of the particularly favoured form of the polynucleotide, peptide or protein, vector, antibody, cell, compound, active ingredient and/or an active ingredient which binds to a peptide or protein selected from the following group for the production of a medicament for the treatment of pain:

JNK3, PIM-2, LR-11, TFIIFβ, GGT-β, GATA3, CLC-7, catalase, tetraspanin (TM4SF/TSPAN-6/TM4-D), casein kinase 1a, MAP3K7/TAK-1 (TGF-β activated kinase), BAB14112 and/or spermidine synthase,

a peptide or protein which, or a part of which, is encoded by a polynucleotide according to any of FIGS. 34a), 34c), 34e), 34g), 35a), 35c), 35e), 36a), 36c), 37a), 37c), 38a), 38c), 39a), 39c), 39e), 40a), 40c), 40e), 41a), 41c), 41e), 42a), 42e), 43c), 43e), 44e), 45a), 45c), 45e), or 46e) or a polynucleotide at least 90%, preferably 95%, in particular 97% similar thereto, or

a peptide or protein having an amino acid sequence according to any of FIGS. 34b), 34d), 34f), 34h), 35b), 35d), 35f), 36b), 36d), 37b), 37d), 38b), 38d), 39b), 39d), 40b), 40d), 40f), 41b), 41d), 41f), 42b), 42f), 43d), 43f), 44f), 45b), 45d), 45f) or 46f) or a peptide or protein at least 90%, preferably 95%, in particular 97% similar thereto.

A further strongly preferred use for the treatment of pain concerns the first particularly favoured form of the compound and/or an active ingredient which binds to a peptide or protein selected from the following group:

JNK3, PIM-2, LR-11, TFIIFβ, GGT-β, GATA3, CLC-7, catalase, tetraspanin (TM4SF/TSPAN-6/TM4-D), casein kinase 1a, MAP3K7/TAK-1 (TGF-β activated kinase), BAB14112 and/or spermidine synthase,

a peptide or protein which, or a part of which is encoded by a polynucleotide according to any of FIGS. 34a), 34c), 34e), 34g), 35a), 35c), 35e), 36a), 36c), 37a), 37c), 38a), 38c), 39a), 39c), 39e), 40a), 40c), 40e), 41a), 41c), 41e), 42a), 42e), 43c), 43e), 44e), 45a), 45c), 45e), or 46e) or a polynucleotide at least 90%, preferably 95%, in particular 97% similar thereto, or

a peptide or protein having an amino acid sequence according to any of FIGS. 34b), 34d), 34f), 34h), 35b), 35d), 35f), 36b), 36d), 37b), 37d), 38b), 38d), 39b), 39d), 40b), 40d), 40f), 41b), 41d), 41f), 42b), 42f), 43d), 43f), 44f), 45b), 45d), 45f) or 46f) or a peptide or protein at least 90%, preferably 95%, in particular 97% similar thereto.

A further strongly preferred use for the treatment of pain concerns the second particularly favoured form of the compound and/or the particularly favoured form of the active ingredient.

Use of a polynucleotide, peptide or protein, vector, antibody and/or cell according to the invention for gene therapy. It is particularly preferred if it is in vivo or in vitro gene therapy. By gene therapy a form of therapy is understood in which, by introducing nucleic acids into cells, an effector gene, usually a protein, is expressed. In vivo and in vitro methods differ in principle. In in vitro methods cells are removed from the organism and transfected with vectors ex vivo before being reintroduced into the same or into another organism. In in vivo gene therapy, in the fight against tumours, for example, vectors are applied systemically (e.g. via the bloodstream) or directly into the tumour.

Said use is particularly preferred if the polynucleotide, in particular the particularly favoured form thereof the second particularly favoured form of the polynucleotide defined by the production process is used.

Also preferred for application in gene therapy is the use of the first particularly favoured form of the polynucleotide.

Also preferred for application in gene therapy is the use of a polynucleotide when it is an antisense polynucleotide or PNA showing a sequence capable of binding specifically to a polynucleotide according to the invention, or which is part of a ribozyme or other DNA enzyme or of a catalytic RNA or DNA.

The invention further relates to the use of a polynucleotide, peptide or protein, vector, antibody, cell, compound, and/or an active ingredient according to the invention for diagnosis and/or for studies of effectiveness. By diagnosis the analysis of symptoms belonging to an illness is understood, and by studies of effectiveness studies concerning the effectiveness of substances to be tested are meant, in particular their medicinal effectiveness.

The invention further relates to another screening process, namely a method with the following process steps for discovering pain-regulating substances:

(a) incubation of a substance to be tested under appropriate conditions with a cell and/or a preparation from such a cell which has synthesised a peptide or protein encoded by a polynucleotide defined by the production method, preferably the second particularly favoured form of the polynucleotide or a polynucleotide at least 90%, preferably 95%, in particular 97% similar thereto.

(b) measurement of the binding of the test substance to the peptide or protein synthesised by the cell, or measurement of at least one of the functional parameters altered by the binding of the test substance to the peptide or protein.

It is preferred if the cell is genetically manipulated before step (a). It is particularly preferred if the genetic manipulation allows measurement of at least one of the functional parameters altered by the test substance. It is also particularly preferred if the cell is genetically manipulated in such a way that the cell contains at least one polynucleotide defined by the production method, preferably the second particularly preferred form of the polynucleotide or a polynucleotide at least 90%, preferably 95%, in particular 97% similar thereto.

A further preferred embodiment of the method is if the cell is cultivated, optionally under selection pressure, after the genetic manipulation and before step (a), under conditions which allow expression.

The invention further relates also to a method for producing a peptide or protein according to the invention in which a cell 33-35 according to the invention containing a polynucleotide or vector according to the invention, is cultivated and, optionally, the peptide or protein is isolated.

The invention further relates to the use of a peptide or protein selected from any of the following groups:

JNK3, PIM-2, LR-11, TFIIFβ, GGTβ-, GATA3, CLC-7, catalase, tetraspanin (TM4SF/TSPAN-6/TM4-D), casein kinase 1a, MAP3K7/TAK-1 (TGF-β activated kinase), BAB14112 and/or spermidine synthase,

a peptide or protein, for which or for a part of which a polynucleotide codes according to any of FIGS. 34a), 34c), 34e), 34g), 35a), 35c), 35e), 36a), 36c), 37a), 37c), 38a), 38c), 39a), 39c), 39e), 40a), 40c), 41a), 41c), 41e), 42a), 42e), 43c), 43e), 44e), 45a), 45c), 45e), or 46e) or for which a polynucleotide codes which is at least 90% similar thereto

a peptide or protein having an amino acid sequence according to any of FIGS. 34b), 34d), 34f), 34h), 35b), 35d), 35f), 36b), 36d), 37b), 37d), 38b), 38d), 39b), 39d), 40b), 40d), 40f), 41b), 41d), 41f), 42b), 42f), 43d), 43f), 44f), 45b), 45d), 45f) or 46f) or a peptide or protein at least 90% similar thereto

and/or a protein, which is encoded by a nucleic acid which, under stringent conditions, binds to a polynucleotide according to any of FIGS. 34a), 34c), 34e), 34g), 35a), 35c), 35e), 36a), 36c), 37a), 37c), 38a), 38c), 39a), 39c), 39e), 40a), 40c), 40e), 41a), 41c), 41e), 42a), 42e), 43c), 43e), 44e), 45a), 45c), 45e), or 46e), or the antisense thereof

a partial protein at least 10 amino acids long, of any of the aforementioned proteins and/or peptides and/or

a peptide or protein encoded by a a gene consisting of a polynucleotide, whose nucleotide sequence comprises a gene fragment according to any of the sequences represented in FIGS. 1-33 or by a polynucleotide which is at least 90% similar to such a gene.

The invention further relates to a polynucleotide corresponding by at least 90%, preferably 95%, in particular 97% or precisely to the nucleotide sequence represented in FIG. 35e).

The invention further relates to a protein corresponding by at least 90%, preferably 95%, in particular by at least 97% or precisely to the amino acid sequence represented in FIG. 35f).

The invention further relates to a method for the treatment, in particular the treatment of pain, of a non-human mammal or of a human being in need of treatment for pain, in particular chronic pain, by administering a substance which binds to a protein or peptide selected from the following group:

JNK3, PIM-2, LR-11, TFIIFβ, GGT-β, GATA3, CLC-7, catalase, tetraspanin (TM4SF/TSPAN-6/TM4-D), casein kinase 1a, MAP3K7/TAK-1 (TGF-β activated kinase), BAB14112 and/or spermidine synthase,

a peptide or protein, for which or for a part of which a polynucleotide codes according to any of FIGS. 34a), 34c), 34e), 34g), 35a), 35c), 35e), 36a), 36c), 37a), 37c), 38a), 38c), 39a), 39c), 39e), 40a), 40c), 40e), 41a), 41c), 41e), 42a), 42e), 43c), 43e), 44e), 45a), 45c), 45e), or 46e) or for which a polynucleotide codes which is at least 90% similar thereto

a peptide or protein having an amino acid sequence according to any of FIGS. 34b), 34d), 34f), 34h), 35b), 35d), 35f), 36b), 36d), 37b), 37d), 38b), 38d), 39b), 39d), 40b), 40d), 40f), 41b), 41d), 41f), 42b), 42f), 43d), 43f), 44f), 45b), 45d), 45f) or 46f) or a peptide or protein at least 90% similar thereto

and/or a protein which is encoded by a nucleic acid which binds under stringent conditions to a polynucleotide according to any of FIGS. 34a), 34c), 34e), 34g), 35a), 35c), 35e), 36a), 36c), 37a), 37c), 38a), 38c), 39a), 39c), 39e), 40a), 40c), 40e), 41a), 41c), 41e), 42a), 42e), 43c), 43e), 44e), 45a), 45c), 45e), or 46e), or the antisense thereof

a partial protein at least 10 amino acids long of any of the aforementioned proteins and/or peptides and/or

a peptide or protein for which a gene consisting of a polynucleotide codes, whose nucleotide sequence contains a gene fragment according to any of the sequences represented in FIGS. 1-33 or for which a polynucleotide codes which is at least 90% similar to such a gene.

Administration can take place, for example, in the form of a medicament as described above.

The invention further relates to a method for the treatment, in particular the treatment of pain, of a non-human mammal or of a human being in need of treatment for pain, in particular chronic pain, by administering a medicament according to the invention, in particular one containing a substance according to the invention and/or an active ingredient according to the invention.

All in all an important basis of the invention is the identification of pain-regulated genes and gene fragments. The screening method is based on this. However, it is possible to use the method for diagnosis or treatment as already explained. Corresponding possibilities for use and further embodiments are explained below.

1. Treatment of Chronic Pain

The sequences were isolated from spinal cord tissue. In the spinal cord, the primary sensory neurones project onto central nervous neurones downstream; in addition to supraspinal processes, processes centred round the central synapse for nociceptive information are involved. Numerous experiments have been able to demonstrate that plastic changes in the nervous system are at the origin of the development of chronic pain conditions (for an overview see Corderre et al., 1993; Zimmermann and Herdegn, 1996). Plastic changes which are accompanied by the regulation of pain-relevant genes have been described in particular in the neurones in the dorsal root ganglia and the spinal cord. Thus gene regulation in the spinal cord (see Table 1) has been described for a number of neurotransmitter receptors that are important for pain therapy. On this basis the cDNA sequences found, which are regulated when pain occurs, can be used for treatment (gene therapy, antisense, ribozyme) and diagnosis of chronic pain conditions.

1.1 Antisense Strategies

Here constructs which can reduce the mRNA or protein concentration are established, derived from the nucleic acid sequence of full-length cDNA or partial regions. Said constructs can be, for example, antisense oligonucleotides (DNA or RNA) which demonstrate an increased stability to nucleases, possibly using modified nucleotide components (e.g. 0-allyl ribose). Furthermore, the use of ribozymes which, as enzymatically active RNA molecules, catalyse a specific cleavage of the RNA is conceivable. In addition, vectors that express the sequences according to the invention or express partial regions of said nucleotide sequences under the control of a suitable promoter and are consequently suitable for in-vivo or ex-vivo therapy could also be used. In addition, also possible are antisense constructs that cannot be broken down by endogenous nucleases or can be broken down only slightly thereby when the phosphate backbone of nucleotide sequences (e.g. PNAs, i.e. peptide nucleic acids) is exchanged, or when non-traditional bases, such as inosine, queosine or wybutosine, and likewise such as acetyl-, methyl-, thio- and similarly modified forms of adenine, cytidine, guanosine u, thymidine and uridine are used.

1.2. Antagonists/agonists or Inhibitors/activators of the Gene Products Coded By the Nucleotide Sequences According to the Invention.

This includes substances that by binding to the gene product alter the function thereof These substances can be:

1.2.1. Organic-chemicai molecules which are found in the context of active ingredient screening using as a binding partner the gene products of the cDNA according to the invention.

1.2.2. Antibodies, be they polyclonal, chimeric, single-chain, F_{ab-} fragments or fragments from phage banks, which influence the function specifically, preferably as neutralising antibodies via binding to the gene products.

1.2.3. Aptamers, i.e. nucleic acids or nucleic acid derivatives having protein-binding properties. These also include so-called spiegelmers which are mirror image oligonucleotides obtained by mirror evolution and therefore stable and which can bind a target molecule with high affinity and highly specifically (Kluβmann et al., 1996).

1.3. Gene Therapy

The sequences described can be used for the treatment of neurological disorders, in particular of chronic pain conditions, said sequences being used after cloning into suitable vectors (e.g. adenovirus vectors or adeno-associated virus vectors) for in vivo or ex vivo treatment in order, for example, to induce an overexpression or underexpression of the endogenous gene product, to correct the sequence of the defective gene product (e.g. by transsplicing with the exogenous construct) or to provide a functional gene product.

2. Diagnosis

Polynucelotide sequences (oligonucelotides, antisense DNA & RNA molecules, PNAs) which are derived from the nucleotide sequences according to the invention could be used for the diagnosis of conditions or disorders that are associated with an expression of said gene sequences. Examples of said conditions or disorders include neurological disorders including chronic pain or neuropathic pain (caused, for example, by diabetes, cancer or AIDS) or neurodegenerative disorders such as Alzheimer's disease, Parkinson's disease, Huntington's chorea, Creutzfeld-Jakob disease, amyotrophic lateral sclerosis and dementias. The nucleotide sequences can be used in a variety of ways (Northern blot, Southern blot, FISH analysis, PRINS analysis, PCR) either for the identification of the gene product or of different diagnostically relevant gene products or for the quantification of the gene product. In addition to nucleic acid diagnosis, antibodies or aptamers for the protein encoded by the nucleic acids according to the invention can be used for diagnosis (e.g. using ELISA, RIA, immunocytochemical or immunohistochemical methods), in order to identify the protein or different forms and to quantify the protein.

With respect to a genetic diagnosis, nucleic acid probes derived from he nucleotide sequences according to the invention could be used for the determination of the gene locus (e.g. by FISH, FACS, artificial chromosomes such as YACS, BACs or P1 constructs).

The following examples and figures should explain the invention without restricting it thereto.

### FIGURES AND EXAMPLES

Figures:

FIG. 1) Gene fragment 111_{—}16 [a) originally sequenced gene fragment from DDRT-PCR, b) corresponding 1^{st} lengthened gene fragment, c) corresponding 2^{nd} lengthened gene fragment]

FIG. 2) Gene fragment 111_{—}33 [a) originally sequenced gene fragment from DDRT-PCR, b) corresponding new gene fragment]

FIG. 3) Gene fragment 111_{—}39

FIG. 4) Gene fragment 111_{—}45 [a) originally sequenced gene fragment from DDRT-PCR, b) corresponding lengthened gene fragment]

FIG. 5) Section of gene fragment 113_{—}15

FIG. 6) Section of gene fragment 113_{—}15

FIG. 7) Section of gene fragment 113_{—}15 [a) originally sequenced gene fragment from DDRT-PCR, b) corresponding lengthened gene fragment]

FIG. 8) Gene fragment 113_{—}20

FIG. 9) Gene fragment 124_{—}29 [a) originally sequenced gene fragment from DDRT-PCR, b) corresponding lengthened gene fragment]

FIG. 10) Gene fragment 124_{—}4

FIG. 11) Gene fragment 127_{—}3

FIG. 12) Gene fragment 127_{—}34 [a) originally sequenced gene fragment from DDRT-PCR, b) corresponding 1^{st} lengthened gene fragment, c) corresponding 2nd lengthened gene fragment]

FIG. 13) Gene fragment 127_{—}8 [a) originally sequenced gene fragment from DDRT-PCR, b) corresponding more extensive human nucleotide sequence (RUP), c) upstream part of the corresponding mouse nucleotide sequence, d) downstream part of the corresponding mouse nucleotide sequence, e) upstream part of the corresponding rat nucleotide sequence, f) downstream part of the corresponding rat nucleotide sequence].

FIG. 14) Gene fragment 135_{—}18

FIG. 15) Gene fragment 135_{—}2

FIG. 16) Gene fragment 135_{—}7

FIG. 17) Section of gene fragment 135_{—}9 [a) originally sequenced gene fragment from DDRT-PCR, b) corresponding lengthened gene fragment]

FIG. 18) Section of gene fragment 135_{—}9 [a) originally sequenced gene fragment from DDRT-PCR, b) corresponding lengthened gene fragment]

FIG. 19) Gene fragment 141_{—}1

FIG. 20) Section of gene fragment 141_{—}13 [a) originally sequenced gene fragment from DDRT-PCR, b) corresponding more extensive human nucleotide sequence (related to ABLIM and KIAA 0843), c) corresponding mouse nucleotide sequence, d) corresponding rat nucleotide sequence].

FIG. 21) Section of gene fragment 141_{—}13 [a) originally sequenced gene fragment from DDRT-PCR, b) corresponding lengthened gene fragment]

FIG. 22) Gene fragment 141_{—}24 [a) originally sequenced gene fragment from DDRT-PCR, b) corresponding lengthened gene fragment]

FIG. 23) Gene fragment 141_{—}6

FIG. 24) Gene fragment 146_{—}10

FIG. 25) Gene fragment 154_{—}26

FIG. 26) Gene fragment 154_{—}29

FIG. 27) Gene fragment 164_{—}12

FIG. 28) Section of gene fragment 164_{—}24

FIG. 29) Section of gene fragment 164_{—}24

FIG. 30) Section of gene fragment 164_{—}24

FIG. 31) Gene fragment 180_{—}12

FIG. 32) Gene fragment 180_{—}8

FIG. 33) Gene fragment 89_{—}28

FIG. 34a) cDNA-sequence of JNK3, human; α1-subtype; AN: U34820

FIG. 34b) Amino acid sequence of JNK3, human, α1-subtype; AN: U34820

FIG. 34c) cDNA sequence of JNK3, human, α2-subtype; AN: U34819

FIG. 34d) Amino acid sequence of JNK3, human, α1-subtype; AN: U34819

FIG. 34e) cDNA sequence of JNK3, mouse; AN: AB005665

FIG. 34f) Amino acid sequence of JNK3, mouse; AN: AB005665

FIG. 34g) cDNA sequence of JNK3, rat; AN: NM_{—}012806

FIG. 34h) Amino acid sequence of JNK3, rat; AN: NM_{—}012806

FIG. 35a) cDNA sequence of PIM-2, human; own clone

FIG. 35b) Amino acid sequence of PIM-2, human; own clone

FIG. 35c) cDNA sequence of PIM-2, mouse; AN: L41495

FIG. 35d) Amino acid sequences of PIM-2, mouse; AN: L41495 various protein lengths:

1.) 40 kDa

2.) 37 kDa

3.) 34 kDa

FIG. 35e) cDNA sequence of PIM-2, rat; own clone

FIG. 35f) Amino acid sequence of PIM-2, rat; own clone

FIG. 35g) Comparison of protein sequences of the PIM-2 sequence represented in 35b) with the human PIM-2 sequence NM 006875 deposited in the gene library

FIG. 36a) cDNA sequence of LR11, mouse; AN: AB015790

FIG. 36b) Amino acid sequence of LR11, mouse; AN: AB015790

FIG. 36c) cDNA sequence of LR11, human; AN: Y08110

FIG. 36d) Amino acid sequence of LR11, human; AN: Y08110

FIG. 37a) cDNA sequence of TFIIFβ, rat; AN: D10665

FIG. 37b) Amino acid sequence of TFIIFβ, rat; AN: D10665

FIG. 37c) cDNA sequence of TFIIFβ, human; AN: X59745

FIG. 37d) Amino acid sequence of TFIIFβ, human; AN: X59745

FIG. 38a) cDNA sequence of GGT, rat; AN: L24116

FIG. 38b) Amino acid sequence of GGT, rat; AN: L24116

FIG. 38c) cDNA sequence of GGT, human; AN: L25441

FIG. 38d) Amino acid sequence of GGT, human; AN: L25411

FIG. 39a) cDNA sequence of GATA3, mouse; AN: NM_{—}008091

FIG. 39b) Amino acid sequence of GATA3, mouse; AN: NM_{—}008091

FIG. 39c) cDNA sequence of GATA3, human; AN: NM_002051

FIG. 39d) Amino acid sequence of GATA3, human; AN: NM_{—}002051

FIG. 39e) Partial-cDNA sequence of GATA3, rat; AN: AB000217

FIG. 40a) cDNA sequence of CCL-7, human; AN: AF224741

FIG. 40b) Amino acid sequence of CCL-7, human; AN: AF224741

FIG. 40c) cDNA sequence of CCL-7, rat; AN: Z67744

FIG. 40d) Amino acid sequence of CCL-7, rat; AN: Z67744

FIG. 40e) Genomic DNA-sequence of CCL-7, mouse; AN: AH063101

FIG. 40f) Amino acid sequence of CCL-7, mouse; AN: AH063101

FIG. 41a) cDNA sequence of catalase, rat; AN: NM_{—}012520

FIG. 41b) Amino acid sequence of catalase, rat; AN: NM_{—}012520

FIG. 41c) cDNA sequence of catalase, mouse; AN: NM_{—}009804

FIG. 41d) Amino acid sequence of catalase, mouse; AN: NM_{—}009804

FIG. 41e) cDNA sequence of catalase, human; AN: NM_{—}001752

FIG. 41f) Amino acid sequence of catalase, human; AN: NM_{—}001752

FIG. 42a) cDNA sequence of casein kinase 1α, rat; AN: U77582

FIG. 42b) Amino acid sequence of casein kinase 1α, rat; AN: AAB19227

FIG. 42e) cDNA sequence of casein kinase 1α, human; AN: NM_{—}001892

FIG. 42f) Amino acid sequence of casein kinase 1α, human; AN: NM_{—}001892

FIG. 43c) cDNA sequence of tetraspanin-6, mouse; AN: AF053454

FIG. 43d) Amino acid sequence of tetraspanin-6, mouse; AN: AAC69711

FIG. 43e) cDNA sequence of tetraspanin TM4-D, human; AN: AF133426

FIG. 43f) Amino acid sequence of tetraspanin TM4-D, human; AN: AF133426

FIG. 44e) cDNA sequence of MAP3K7/TAK-1, human; AN: NM_{—}003188

FIG. 44f) Amino acid sequence of MAP3K7/TAK-1, human;. AN: NM_{—}003188

FIG. 45a) cDNA sequence of spermidine synthase, rat; ANT: AF337636

FIG. 45b) Amino acid sequence of spermidine synthase, rat; AN: AAK21288

FIG. 45c) cDNA sequence of spermidine synthase, mouse; AN: L19311

FIG. 45d) Amino acid sequence of spermidine synthase, mouse; AN: AAC37666

FIG. 45e) cDNA sequence of spermidine synthase, human; AN: XM_{—}042276

FIG. 45f) Amino acid sequence of spermidine synthase, human; AN: XP_{—}042276

FIG. 46e) cDNA sequence of BAB14112, human; AN: AK022582

FIG. 46f) Amino acid sequence of BAB14112, human; AN: BAB14112

FIG. 47) Casein-kinase in various pain models

FIG. 48) Tetrapanin (TSPAN) in various pain models

FIG. 49) M3K7-kinase in various pain models

FIG. 50) Overview of the general cloning strategy used

FIG. 51) Overview of the regulation of the identified genes and gene fragments

### EXAMPLES

### Example 1

### Identification, Isolation and Sequencing of Pain-regulated Genes

1.) Procedure

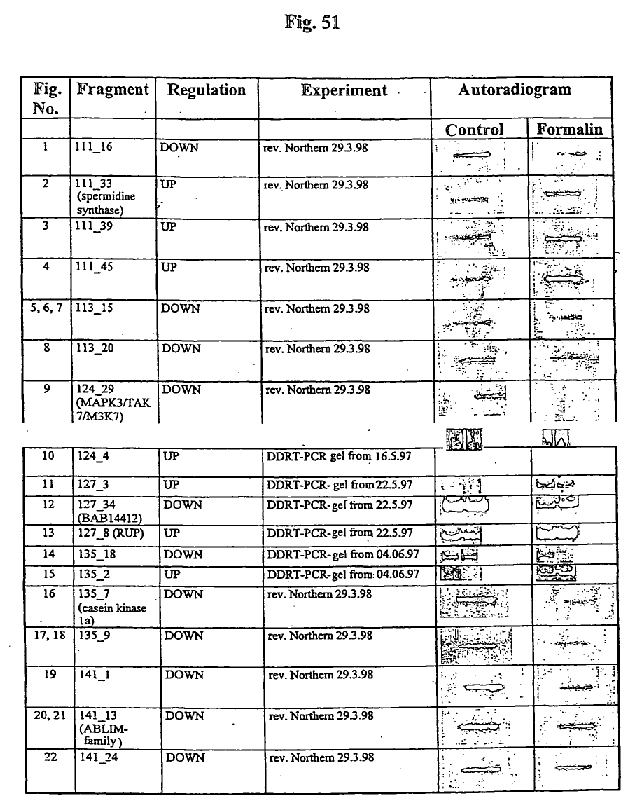

The following procedure was selected (for explanation see FIG. 50):

As the starting point for the isolation of pain-regulated genes, the so-called formalin model in the rat was selected, wherein formalin is injected into the rat's paw. The target tissue in which the pain-regulated expression of the genes according to the invention was demonstrated was the dorsal section of the rat's spinal cord in segments L3-L6. Four methods are available for the isolation of differentially regulated genes:

cDNA-RDA (cDNA-representational difference analysis; Hubank & Schatz, 1994)

DDRT-PCR (Differential Display RT-PCR; Liang & Pardee 1992, Bauer et al., 1994),

Subtractive hybridisation (Watson & Margulies, 1993)

SAGE (Serial Analysis of Gene expression, Velculescu et al., 1995).

A comparative evaluation of the above methods led to the selection of DDRT-PCR, since said method, in contrast to subtractive hybridisation and SAGE, is capable of including both up- and down-regulated genes and also rare transcripts and, over and above this, yields an abundance of results within short timespans.

2.) Material and Methods

Isolation and Characterisation of Pain-regulated cDNA- Sequences

Method according to Liang & Pardee (1992)/U.S. Pat. No. 5,262,311 Modifications:

1. Decamer-primer according to Bauer at al. 1993

2. OligoDT-primer mixtures according to Sompayrac et al. 1995

3. TAE-polyacryl amide-gel electrophoresis

TABLE 2

| | |
|---|---|
| Name | Sequence (in 5′=>3′-orientation) |

Oligonucleotide primers used

| | |
|---|---|
| Deca1 | TACAACGAGG |
| Deca2 | TGGATTGGTC |
| Deca3 | CTTTCTACCC |
| Deca4 | TTTTGGCTCC |
| Deca5 | GGAACCAATC |
| Deca6 | AAACTCCGTC |
| Deca7 | TCGATACAGG |
| Deca8 | TGGTAAAGGG |
| Deca9 | TCGGTCATAG |
| Deca10 | GGTACTAAGG |
| Deca11 | TACCTAAGCG |
| Deca12 | CTGCTTGATG |
| Deca13 | GTTTTCGCAG |
| Deca14 | GATCAAGTCC |
| Deca15 | GATCCAGTAC |
| Deca16 | GATCACGTAC |
| Deca17 | GATCTGACAC |
| Deca18 | GATCTCAGAC |
| Deca19 | GATCATAGCC |
| Deca20 | GATCAATCGC |
| Deca21 | GATCTAACCG |
| Deca22 | GATCGCATTG |
| Deca23 | GATCTGACTG |
| Deca24 | GATCATGGTC |
| Deca25 | GATCATAGCG |
| Deca26 | GATCTAAGGC |
| T7-PCR | ACGACTCACTATAGGGCGAATTGG |
| M13R-PCR | ACACAGGAAACAGCTATGACCATG |
| T7(dT)₁₅ | AAACGACGGCCAGTGAATTGTAATACGACTCACTATAGGCGC |

T₁₅

oligodT-primer mixtures (equimolar mixtures)

| | |
|---|---|
| 3P1 | T₁₂AA+T₁₂AT+T₁₁AC+T₁₁AG |
| 3P2 | T₁₁GA+T₁₁GT+T₁₁CA+T₁₁CT |
| 3P3 | T₁₀GC+T₁₀GG+T₁₀CC+T₁₀CG |

Animal model: The formalin test represents a suitable model for the region of inflammatory/persistent pain (Duibisson et al., 1997). In said test, 50 μl of 5% formalin solution were injected unilaterally into the hindpaw of adult Wistar-rats and the animals were killed 24 hours after the injection in order to take a tissue sample. Parallel to this, isotonic saline solution was injected into the hindpaw of the control animals.

Taking of tissue samples. The animals are decapitated, the dorsal spinal column is removed and the spinal cord is eluated from the spinal canal by injection of a large quantity of an ice-cold isotonic saline solution. After removal of the dura, the dorsal half of the region from L3 to L6 is cut out and frozen immediately in liquid nitrogen.

RNA-isolation. The total RNA was isolated from the tissue samples using the Trizol-Kit (Life Technologies) according to the manufacturer's instructions. The RNA was quantified using UV-spectrometry (extinction at 260 nm) and its integrity was verified using denaturing gel electropheresis in a formaldehyde-agar gel (Sambrook et al., 1989).

DNase-digestion. Before use in DDRT-PCR, possible traces of genomic DNA were removed by DNase-digestion. In each case, 6 μg RNA were incubated at 37° C. in a total volume of 100 μI in 1× First Strandbuffer (Life Techn.) and 10 Units RNase-free DNasel (Boehringer Mannheim) for 15 minutes. After phenol-chloroform extraction, the RNA was precipitated by adding 1/10 vol. sodium acetate pH 5.2, and 2.5 vol. ethanol, dissolved in DEPC-water, quantified using UV-spectrometry and characterised using formaldehyde agar gel electrophoresis again.

Reverse Transcription. Each 200 μg Dnasel-digested RNA was first denatured using 2.5 μM oligodT-primer mixture (3P1, 3P2 or 3P3) by 5 minutes incubation at 70° C., chilled on ice and then incubated in a total volume of 20 μl reaction mixture for 60 min at 37° C. The reaction mixture contained 1× First-Strandbuffer (Life Techn.), 10 mM DTT, 18.75 μM of each of dATP, dCTP, dGTP, and dTTP, 20 units of RNasin (Promega) and 200 units Superscript II RNaseH⁻-reverse transcriptase (Life Techn.). The enzyme was subsequently inactivated by heating for 5 minutes to 99° C. and the cDNA mixture was stored at −20° C.

DDRT-PCR Each 2 μl of the cDNA were subjected in a volume of 20 μl to the polymerase chain reaction (PCR). The reaction mixture contained 1× PCRII-buffer (Perkin-Elmer), 2.5 mM MgCl₂, 2 μM each of dATP, dCTP, dGTP, and dTTP, 0.2 μM decamer-primer (Decal-26), 0.8 μM oligodT- primer mixture (3P1, 3P2 or 3P3) and 2 μCi [α-³³ P]dATP (≧2000 mCi/mmol, Amersham). The PCR mixtures were subjected to the following temperature profile in a PTC200-thermocycler (MJ Research): 3 Min at 94° C.; 40 cycles of 15 sec at 94° C., 1 min at 40° C., within 70 sec heating up to 72° C., 30 sec at 72° C.; 10 min at 72° C. and storage at 4° C.

Gel electrophoresis and evaluation. The PCR-samples were first concentrated in a vacuum until dried, dissolved in a 4 μl test buffer (0.25% bromphenol blue, 0.250% xylene cyanol FF, 30% glycerine) and each 2 μl section was separated electrophoretically in a 6% tris-taurine-EDTA-polyacryl amide gel (Multiphor-System, Promega) for 2.5 hr. at 50 watts. The gel was subsequently dried for an hour at 80° C. and exposed overnight on a BASIII-detection screen (Fuji). For evaluation, the STORM phosphorus-imager (Molecular Dynamics) was used with ImageQuant-Software. The autoradiographic data were printed in the same scale on film which was then used to cut out the fragments.

Reamplification of the DDRT-PCR-fragments. Differentially regulated PCR bands were cut out of the gel with a scalpel and eluated from the segment of gel by boiling for 10 minutes in 100 μl bidist. water. 25 μl thereof were again amplified in a total volume of 50 μl using PCR. The PCR-reaction mixture contained 1×PCR-Pufferil (Perkin-Elmer); 1.5 mM MgCl₂; 50 μM of each of dATP, dCTP, dGTP, and dTTP; M 0.2 μM of the corresponding decamer; 1 μM of 1PX oligodT-primer mixture and 2.5 μl units of AmpliTAQ-DNA-polymerase (Perkin-Elmer). The PCR temperature profile corresponded with the original PCR-reaction (see above). The PCR mixtures were then mixed with 10 μl test buffer (0.25% bromphenol blue, 0.25% xylene cyanol FF, 30% glycerine) and separated electrophoretically in a 3% TAE agar gel with 10 μg/ml ethidium bromide and PCR products of the expected size were cut out of the gel.

Cloning in TA-cloning vectors. The excised fragments were purified using the Qiaquick gel-extraction kit (Qiagen) according to the manufacturer's instructions, reduced until dried and dissolved in 5 μl bidistilled. water. They were subsequently ligated in the pCRII- TOPO-vector using the TOPO TA Cloning Kit (Invitrogen) according to the manufacturer's instructions and transformed in TOP10F′-E.coli cells. The transformation mixture was plated out with 100 μg/ml ampicillin on LB agar plates which had previously been treated with 50 μl of 2% X-Gal (Sigma) and 50 μl of isopropyl thiogalactoside (Sigma). The white bacterial clones obtained after 15 hours incubation at 37° C. were reduced in 5 ml LB-liquid medium with 100 μg/ml ampicillin (100 μg/ml) and incubated overnight at 37° C. under agitation. Plasmid-DNA was isolated from said cultures using the Qiagen-Spin-Miniprep-Kit (Qiagen) according to the manufacturer's instructions and each 5 μl of the plasmid-DNA was characterised by EcoRI-restriction digestion and subsequent TAE agar gel electrophoresis.

Sequence analysis. Here each 500 ng of the plasmid DNA was sequenced with the T7-PCR primer using the Dye Terminator Cycle Sequencing Kit (Perkin-Elmer) and the reactions were analysed using the automatic sequencer ABI 370 (Applied Biosystems Inc.). The DNA sequences were compared with the gene libraries using bioSCOUT software (LION, Heidelberg).

Confirmation of the differential regulation of the cloned cDNA sequences.

Here the so-called Reverse Northern technique was used, wherein the cDNA fragments were bound onto replica filters after PCR amplification and hybridised using radioactively labelled cDNA from control spinal cord versus formalin spinal cord.

PCR amplification of the cloned cDNA fragments.

Each 50-100 ng of the plasmid DNA was subjected in a volume of 100 μl to the polymerase chain reaction (PCR). The reaction mixture contained 1× PCRII-buffer (Perkin-Elmer), 1.25 mM MgCl₂, 10% DMSO, 250 μM each of dATP, dCTP, dGTP, and dTTP, 0.8 μM T7 PCR primer, 0.8 μM M13R-PCR primer and 5 units Ampli-TAQ-DNA polymerase (Perkin-Elmer). The PCR mixtures were subjected to the following temperature profile in a PTC200-thermocycler (MJ Research): 3 min at 94° C.; 40 cycles of 15 sec at 94° C., 15 sec at 55° C., 30 sec at 72° C., 72° C. and storage at 4° C. A 5 μl aliquot was separated on a 3% TAE agar gel and where the PCR reaction was successful, the PCR mixtures were purified using the Quiaquick PCR Purification Kit (Qiager) according to the manufacturer's instructions and quantifed using WV spectrometry.

Production of the slot blot DNA filter. The Hybond N-filters were first immersed for 10 minutes in 6×SSC buffer and clamped into the slot blot apparatus (Schleicher and Schüll). Each 1 μg of the DNA fragments were diluted in 6×SSC buffers, denatured by boiling for 10 minutes and subsequent chilling on ice and sucked onto the filter. The filters were subsequently washed for 10 min in 1.5M NaCl/0.5M NaoH solution and for 10 min in 1 M NaCl/0.5M tris-HCl pH=7.0, dried at room temperature for 30 min and bound to the filter by 5 minutes UV irradiation (302 nm, Transilluminator).

Production of amplified RNA. (modified according to Poirier et al., 1997 & Superscript Choice System. Life Techn.). Here 5 ng of total-RNA were denatured with 100 ng T7 (dT) 15 primer in a volume of 11 μl for 5 min at 70° C. and chilled on ice. The denatured RNA was then incubated for 1 hour at 42° C. in a total volume of 20 μl with 1×First-Strand buffer (Life Techn.), 10 mM Dtt, 500 μM each of dATP, dCTP, dGTP, and dTTP and 400 units Superscriptil reverse transcriptase (Life Techn.), and cooled to 4° C. For 2nd-strand synthesis, the following were added thereto: 91 μl DEPC-H₂O, 30 μl 2nd-Strandbuffer (Life Tech.), 3 μl 10 mM dNTPs (Boehringer Mannheim), 1 μl E.coli DNA-ligase (10 U/μl; Life Tech.), 4 μl E.coli DNA-polymerase I (10 U/μl Life Tech.), 1 μl Rnase H (2 U/μl; Life Tech.), the mixture was then incubated for 2 hours at 16° C. and after addition of 2 μl T4 DNA-polymerase (5 U/μl; Life Tech.) incubated for a further 5 min at 16° C. Inactivation ensued by adding 10 μl 0.5 M EDTA and 10 min heating to 65° C. After phenol-chloroform-extraction and precipitation using 70 μl 7.5 M NH₄OAc and 500 μl 100% cold ethanol (−20° C.), the precipitate was centrifuged off (5 min 14000 g, RT), washed with 1 ml 70% ethanol, dissolved in 50 μl DEPC-H₂O, and desalinated using a S200-Sephacryl column (Microspin; Pharmacia). An aliqout (2 μl) is quantified using UV-spectrometry. RNA is produced from said double-stranded cDNA by means of in vitro transcription, 50 μl of cDNA (5-10 μg) being incubated in a total volume of 100 μl with 1×transcription buffer (Promega), 7.5 mM rNTPs, 10 μl T7 RNA Polymerase-Mix (Ribomax-Kit; Promega) for 3-4 hours at 37° C. and subsequently cooled to 4° C. After addition of 3 μl RQ1-DNase (Rnase-free; Promega), the mixture is incubated for 15 min at 37° C. After phenol-chloroform-extraction and desalination using a S200-Sephacryl column (Microspin; Pharmacia), an aliqout (2 μl) is quantified using UV-spectrometry.

Production of the radioactive cDNA probe. Each 2 μg of the amplified RNA is denatured using 0.8 μl of the hexamer primer mixture (=750 ng; Boehringer, Mannheim) by heating for 5 minutes to 70° C. and subsequent chilling on ice, and reverse transcribed in a total volume of 25 μl, 2.5 μl of PCR-buffer (Life Tech.); 2.5 μl of 25 mM MgCl₂; 2.5 μl of 0.1 M DTT; and 1 μl each of 20 mM dATP/dTTP/dGTP; 1 μl of 120 μM dCTP and 5 μl [α-³²P] dCTP being added. After 5 minutes incubation at room temperature, 2 μl Superscriptil-reverse transcriptase (200 U/μl, Life Tech.) were added, and the mixture was incubated for 10 min at 25° C. and subsequently for 50 min at 42° C. After heat-inactivation (15 min at 70° C.) the sample was topped up with DEPC water to 50 μl and the non-incorporated radioactivity was removed using an S200-Sephacryl column (Microspin; Pharmacia). The activity of each 0.5 μl of the eluate was determined in a beta-counter (LKB) by means of Czerenkow radiation. In the remainder of the mixture the RNA was hydrolysed by addition to 6 μl 1 M NaOH/10 mM EDTA-solution and 20 minutes incubation at 68° C., subsequently neutralised by addition to 60 μl 1 M phosphate buffer pH=7.0 and added in each case to the prehybridised replica filters.

Prehybridisation, hybridisation, washing and evaluation of the filters. The filters are denatured in hybridisation flasks with 10 ml Expresshyb solution (Clontech), and 100 μg/ml of heat-denatured SalmonSperm-DNA (Sigma) are incubated for 20 min at 68° C. After shaking off the prehybridisation mix and adding 5 ml of the same solution, the temperature whereof is 68° C., the above radioactive cDNA probe is pipetted thereto and the filters incubated overnight at 68° C. in a rotating incubator. A plurality of washing steps subsequently ensue with 0.2×SSC/0.1% SDS for 10 min at room temperature; 0.2×SSC/0.1% SDS for 15 min at 42° C. and finally in 0.2×SSC/0.1% SDS for 15 min at 65° C. The filters are welded wet into film and exposed on a BASIII-detection screen (Fuji). For the evaluation, the STORM phosphorus imager (Molecular Dynamics) was used in conjunction with ImageQuant-Software.

3.) Results

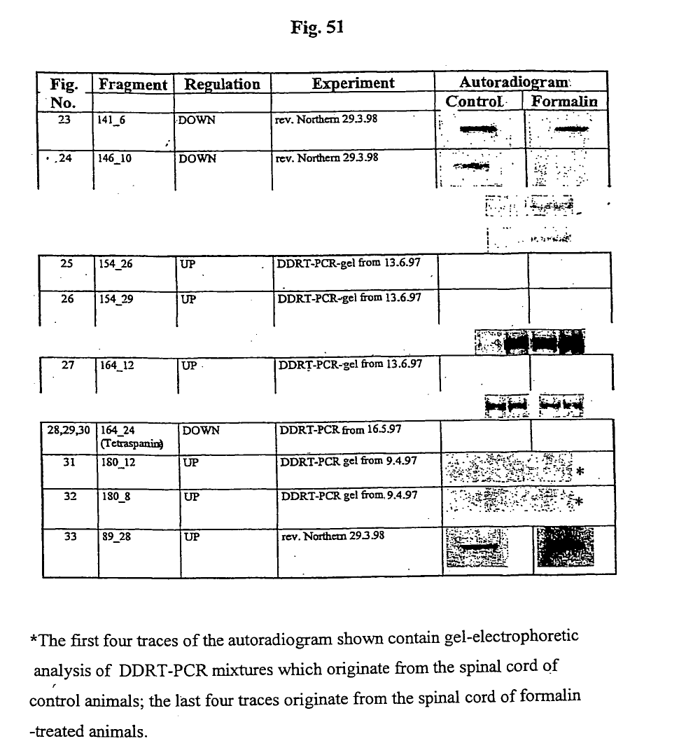

Using said method (see FIG. 50) 35 pain-regulated genes (see Table 3 and FIG. 51) were cloned first as a partial sequence, and, in the further development of the working process, in some cases as a lengthened sequence.

Using the selected animal model as the point of departure, cDNA sequences were cloned from the rat using the cloning strategy applied.

A total of 27 cDNA sequences hitherto unknown from the prior art and which have not been allocated and 8 known cDNA sequences or gene fragments were cloned from the spinal cord of rats, the expression of said sequences being regulated by conditions of chronic pain (see Table 4—cDNA sequences hitherto unknown from the prior art and which have not been allocated—and Table 5—known cDNAs). With regard to the 27 cDNA sequences that are unknown and which have not been allocated, there is either nothing known in the prior art about the function thereof or in some cases something has become known only as a result of a more in depth analysis. Our studies now confirm that they have a role in pain events. An analysis of the expression pattern in various rat tissues showed, however, a focusing on neuronal tissue in the case of 4 cDNAs (111.39, 111.45, 141.13, 154.29; see Table 5). Said cDNAs are thus particularly interesting for the development of new diagnostic and treatment approaches in chronic pain. The role of the novel cDNAs can be further studied by experiments with antisense oligos or ribozymes.

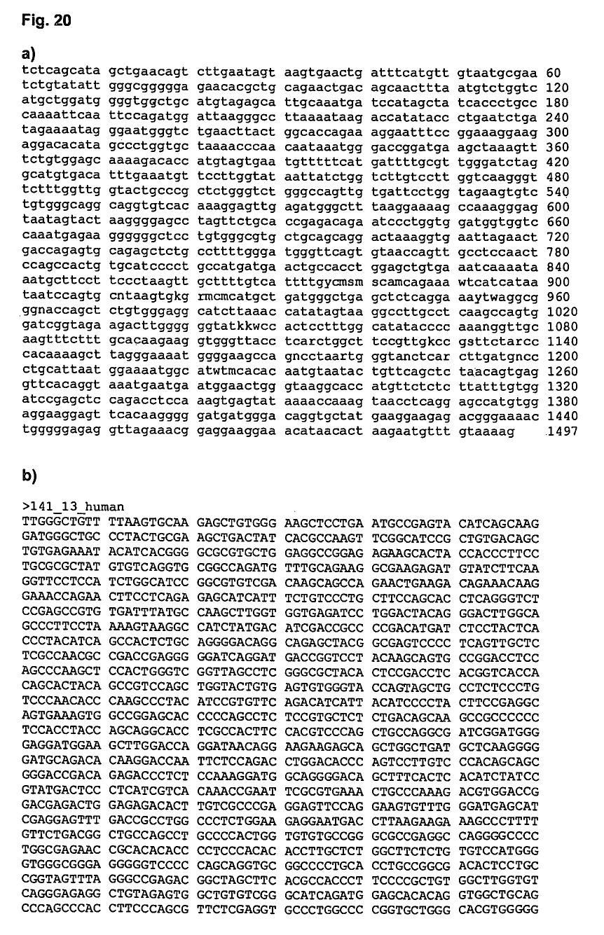

Of particular interest is the gene fragment 141-13. The extension of the gene fragment represented in FIGS. 20 and 21 showed in the analysis that a new gene, which is homologous to UNC-115 and other members of the abLIM family but is hitherto unknown, is present here. Together with the expression pattern and considering the interesting role played by UNC-115 in neuronal development, said gene fragment or gene is therefore of particular interest.

Also of interest is gene fragment 127-8, since said fragment is highly homologous to an obviously highly expressed part of the human genome (AN: NT_{—}009379. 1 on chromosome 11q13.

For the 8 known cDNAs the functions are known in principle:

cDNAs having amplified expression under pain:

Geranylgeranyltransferase

Catalase

Transcription factor TFIIFβ

Transcription factor GATA-3

LDL-like receptor LR-11

Cl-Channel CIC7

cDNAs having reduced expression under pain:

MAP-kinase/JNK3/SAPKβ

PIM-2 kinase

The cDNAs discovered for the gene fragments (GFs) during the more precise analysis are

GF111.33=spermidine syiithase (up-regulated),

GF124-29=MAPK3/TAK-1 (down-regulated),

GF127-34=BAB14112 (down-regulated),

GF135-7=casein kinase 1 a (down-regulated) and

GF164-24=tetraspanin (TM4-D/TSPAN-6) (down-regulated).

Special emphasis should be given here to PIM-2 kinase. For other isotypes of said enzyme, PIM-1 and -3, it was possible to show that said signalling-kinase is involved in learning and memory processes in the hippocampus (Konietzko et al. (1999) EMBO J., 18: 3359-3369).

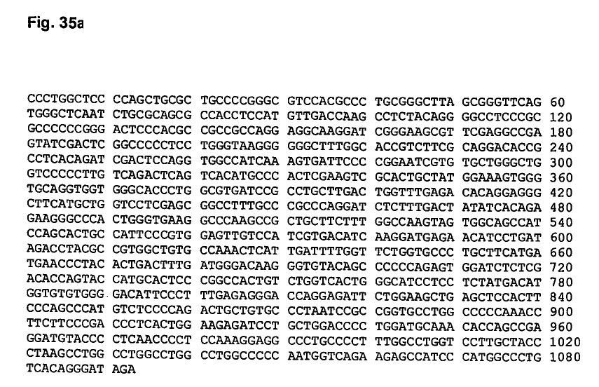

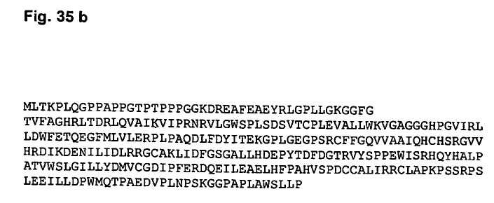

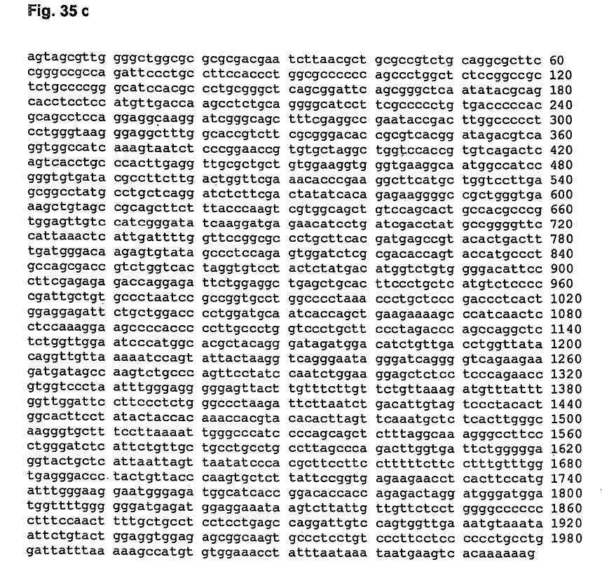

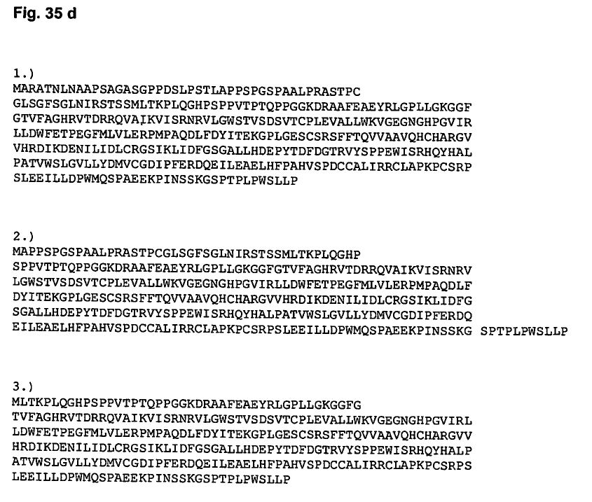

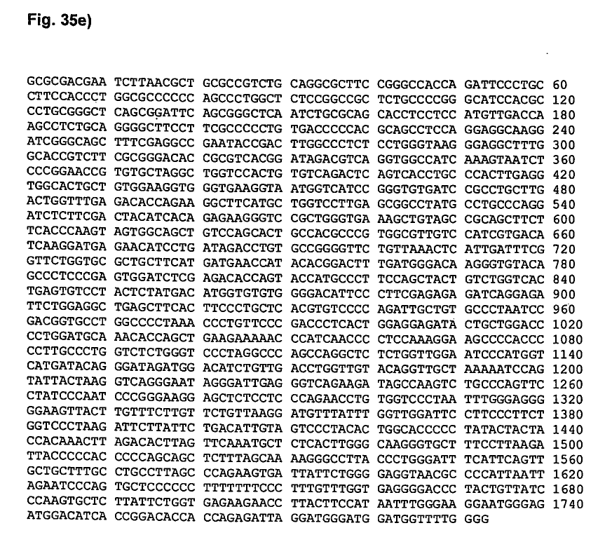

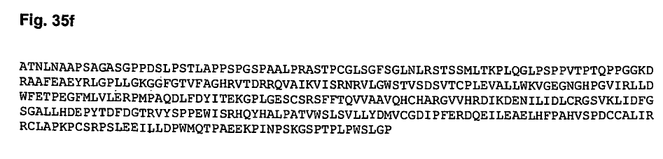

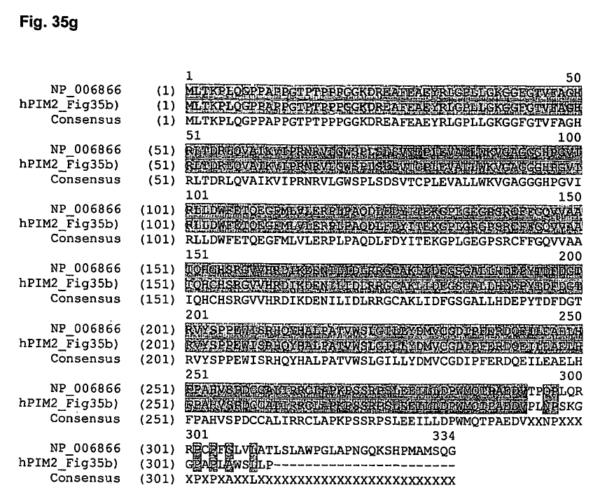

Within the scope of the invention a nucleic acid sequence which encodes the full-length rat PIM-2 protein was isolated and characterised (FIG. 35e, 35f). The nucleic acid sequence and the protein sequence for Pim-2 from the rat are hitherto unknown. Furthermore, a full-length, i.e. containing the total encoded region, cDNA was isolated which encodes the human Pim-2 (35a,b). A comparison of the sequence with the published human PIM-2 sequence (Acc. no. U77735) resulted in the following differences:

| Differences | | |
|---|---|---|
| in sequence | Effect | Interpretation |
| *Nt 230: C→T | Thr→Thr | silent mutation, poss. |
| | | polymorphism |
| Nt 515: A→G | Glu→Glu | silent mutation, poss. |
| | | polymorphism |
| Nt 1064: T deletion | Alteration of the reading | new C-terminal is more |
| | frame leads to the altered | similar to mouse and rat |
| | C-terminal protein | sequence than the |
| | sequence | published human PIM-2 |
| | | sequence |
| Nt 1175: A→C | 3′UTR, no effect on | |
| | protein sequence | |

*The designations of the positions relate to the published Pim-2 sequence (Acc.no. U77735).

Of said differences the deletion of a nucleotide found at position 1064 is of functional relevance since it leads to an alteration in the reading frame and thus results in a protein having a completely different C-terminal protein sequence which is set out below.

Comparison of the C-terminal ends of the two PIM-2 proteins deduced (for a full comparison see FIG. 35g).

hPIM-2 (Acc.no.U77735)

TPQPLQRRPCPFGLULATLSLAWPGLAPNGQKSHPNAMSQG

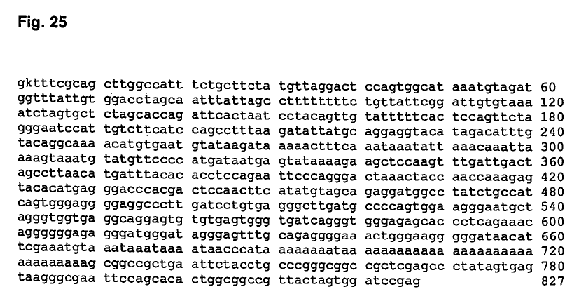

hPIM-2 (Patent FIG. 25b). PLNPSKGGPAPLAWSLLP

A further example is catalase, which removes the H₂O₂ that is harmful to the cell by converting it into O₂ and H₂O.

The protein kinase JNK-3 is specifically expressed in nerve cells of the central nervous system (Mohit et al., 1995) and for a number of opiate receptors (such as, for example, μ-opiate receptor and ORL-1) the downstream activation of protein kinases from said family of mitogen-activated protein kinases has been described (Li & Chang, 1996; Hawes et al., 1998).

The protein LR11 is a mosaic protein of the “low density lipoprotein receptor (LDLR)” family (Möwald et al., 1997). The strongest expression of LR11-mRNA was determined in the brain, said expression furthermore being regulated depending on development in a manner that suggests a function in synaptogenesis or neurite growth (Hermann-Borgmeyer et al., 1998).

TABLE 3
Expression of the 27 (mostly) unknown cDNAs in the adult rat

| | | Expression profile of the novel cDNAs in |
|---|---|---|
| Clone/Fig. No. | Regulation | tissues of the adult rat |
| 180.12/31 | up | remains open |
| 180.8/32 | up | remains open |
| 111.16/1 | down | spleen, stomach, spinal cord |
| 111.33/2 | up | kidney, lung, colon, stomach, brain, spinal |
| | | cord |
| 111.39/3 | up | brain, spinal cord |
| 111.45/4 | up | brain, spinal cord (ubiquitous) |
| 113.15/5, 6, 7 | down | ubiquitous |
| 113.20/8 | down | brain, heart, spleen, spinal cord |
| 124.29/9 | down | colon, brain, lung, testis |
| 124.4/10 | up | ubiquitous |
| 127.3/11 | up | specific to testicles |
| 127.34/12 | down | remains open |
| 127.8/13 | up | spleen, spinal cord (ubiquitous) |
| 135.18/14 | down | remains open |
| 135.2/15 | up | remains open |
| 135.7/16 | down | kidney (ubiquitous) |
| 135.9/17, 18 | down | remains open |
| 141.1/19 | down | Unsuccessful |
| 141.13/19, 20 | down | spinal cord (ubiquitous) |
| 141.24/22 | down | liver, spinal cord, skeletal muscle |
| 141.6/23 | down | Ubiquitous |
| 146.10/24 | down | colon, heart, stomach, spinal cord |
| | | (ubiquitous) |
| 154.26/25 | up | kidney, liver, lung |
| 154.29/26 | up | specific to spinal cord |
| 164.12/27 | up | remains open |
| 164.24/28, 29, 30 | down | remains open |
| 89.28/33 | up | ubiquitous (colon, brain, lung, spinal cord, |
| | | testis) |

Sequence data for the regulated clone

The following table allows the figures to be allocated to the cDNA-clones/gene fragments that are listed in tabular form.

TABLE 4
Hitherto unknown gene fragments that have not yet been allocated

| FIG. No. | Name of the cDNA | Length (bp) |
|---|---|---|
| 1 | 111_16 | 1268 |
| 2 | 111_33 | 840 |
| 3 | 111_39 | 1540 |
| 4 | 111_45 | 2025 |
| 5 | 113_15 | 1721 |
| 6 | 113_15a | 1009 |
| 7 | 113_15b | 685 |
| 8 | 113_20 | 860 |
| 9 | 124_29 | 1348 |
| 10 | 124_4 | 923 |
| 11 | 127_3 | 741 |
| 12 | 127_34 | 109 |
| 13 | 127_8 | 1508 |
| 14 | 135_18 | 1339 |
| 15 | 135_2 | 177 |
| 16 | 135_7 | 1102 |
| 17 | 135_9a | 478 |
| 18 | 135_9b | 616 |
| 19 | 141_1 | 236 |
| 20 | 141_13a | 1497 |
| 21 | 141_13b | 197 |
| 22 | 141_24 | 2818 |
| 23 | 141_6 | 539 |
| 24 | 146_10 | 1759 |
| 25 | 154_26 | 827 |
| 26 | 154_29 | 1081 |
| 27 | 164_12 | 195 |
| 28 | 164_24a | 538 |
| 29 | 164_24b | 547 |
| 30 | 164_24d | 250 |
| 31 | 180_12 | 90 |
| 32 | 180_8 | 559 |
| 33 | 89_28 | 445 |

TABLE 5
Gene fragments from known

| FIG. no. of the known | | Length of the gene |
|---|---|---|
| DNA sequence | Name of the cDNA | fragment found (bp) |
| 34 a, c, e, g | b141_15 | 2733 |
| 35 a, c, e | b141_2 | 314 |
| 36 a, c | b141_26 | 338 |
| 37 a, c | b151_32 | 633 |
| 38 a, c | b151_38 | 2201 |
| 39 a, c, e | b154_13 | 190 |
| 40 a, c, e | b20_37a | 389 |
| 40 a, c, e | b20_37b | 109 |
| 41 a, c, e | b69_24 | 720 |

### Example 2a

### In-situ Hybridisation of Various Gene Fragments Discovered or of Known Genes

Rats were subjected to various pain models, spinal cord sections were obtained some time after the pain was triggered and the expression of each gene or gene fragment was studied by means of in-situ hybridisation using DNA probes or specific antibodies and compared with control animals.

The models were CFA-induced arthritis (CFA), collagen-induced arthritis (CIA) the formalin-test (formalin), see. D. Dubuisson, S. G. Dennis, Pain 4,161-174 (1977).

Table 5a shows the results

TABLE 5a

| | | Expres- | | | |
|---|---|---|---|---|---|
| | | sion | Regulation | | |
| Name | Location | level | CFA | CIA | Formalin |
| JNK3 | grey matter | high | high | high | high |
| | DH + VH | | VH, low | | |
| | | | DH | | |
| PIM-2 | grey matter | relatively | high | high | high |
| | DH + VH | high | DH down | DH down | DH down |
| GF135.7 | grey matter | low | DH down | | |
| | VH > DH | | | | |
| GF111.39 | grey matter | very low | 0 | | 0 |
| | DH ≧ VH | | | | |
| GF111.45 | grey matter | very low | high | | high |
| | DH >> VH | | DH > VH | | DH > VH |
| GF141.24 | grey matter | very low | | | high |
| | DH >> VH | | | | DH > VH |

DH = dorsal horn
VH = ventral horn

### Example 2b

### Determination Using RT-PCR of the Expression Level of Various Known Genes (TSPAN, Casein-kinase and M3K7-kinase) According to Various Pain Models

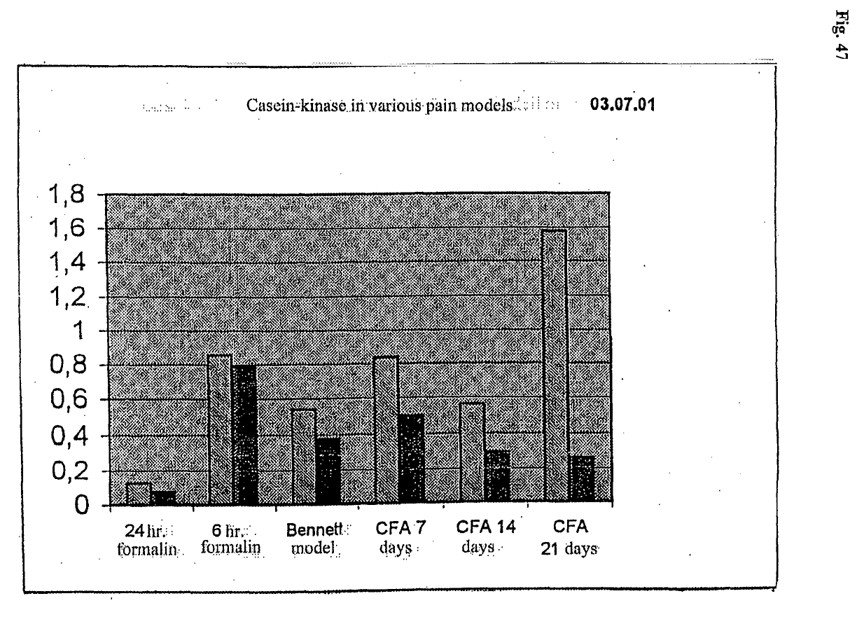

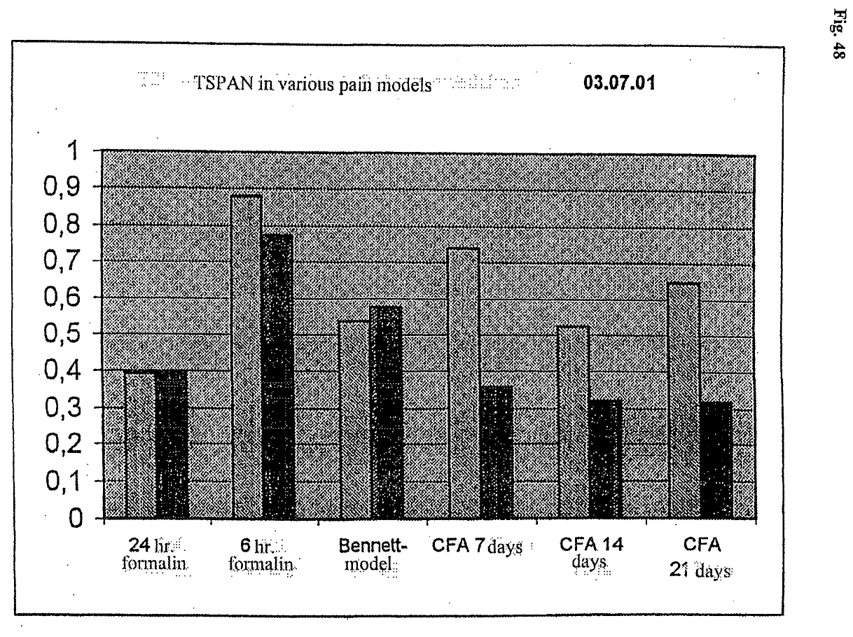

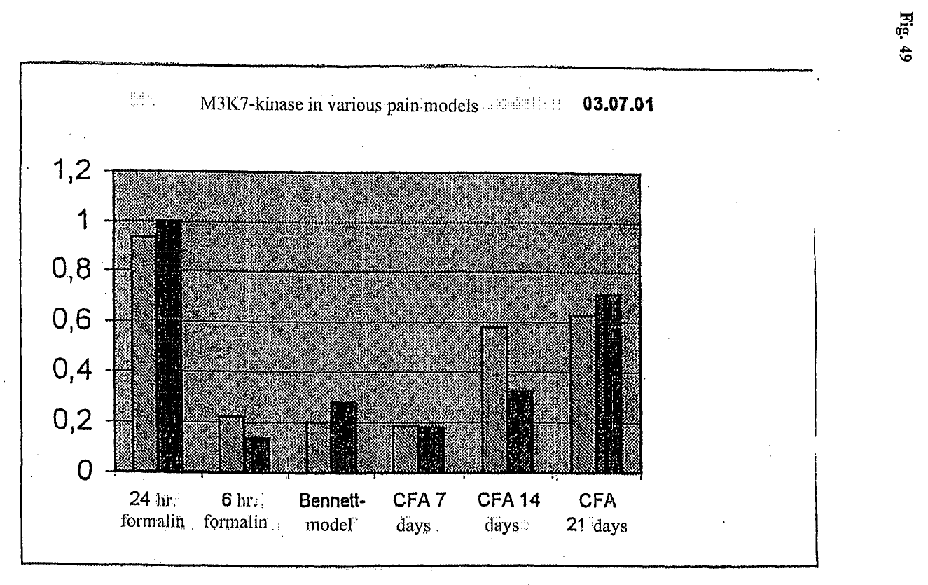

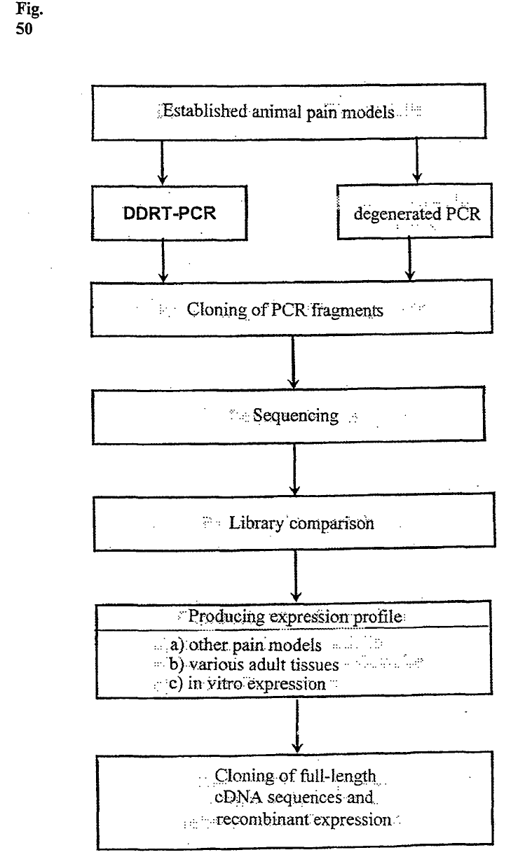

The range of expression according to various pain models compared with controls in rats was determined by RT-PCR (see above). The results can be seen from FIGS. 47, 48 and 49, wherein the light-coloured columns correspond to the control animals and the shaded columns to the treated animals. The y-axes show relative units (standardised expression level)

### Example 2c

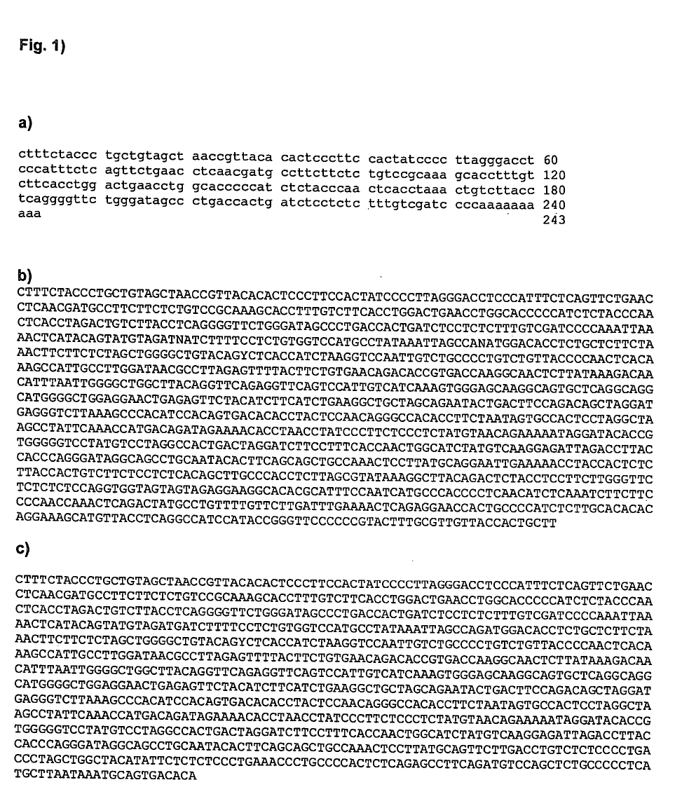

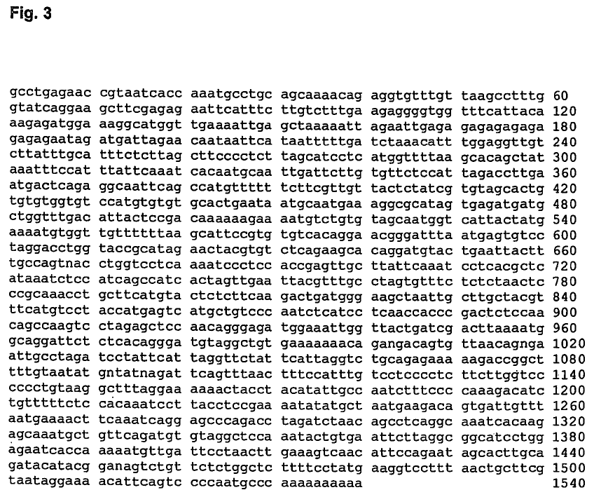

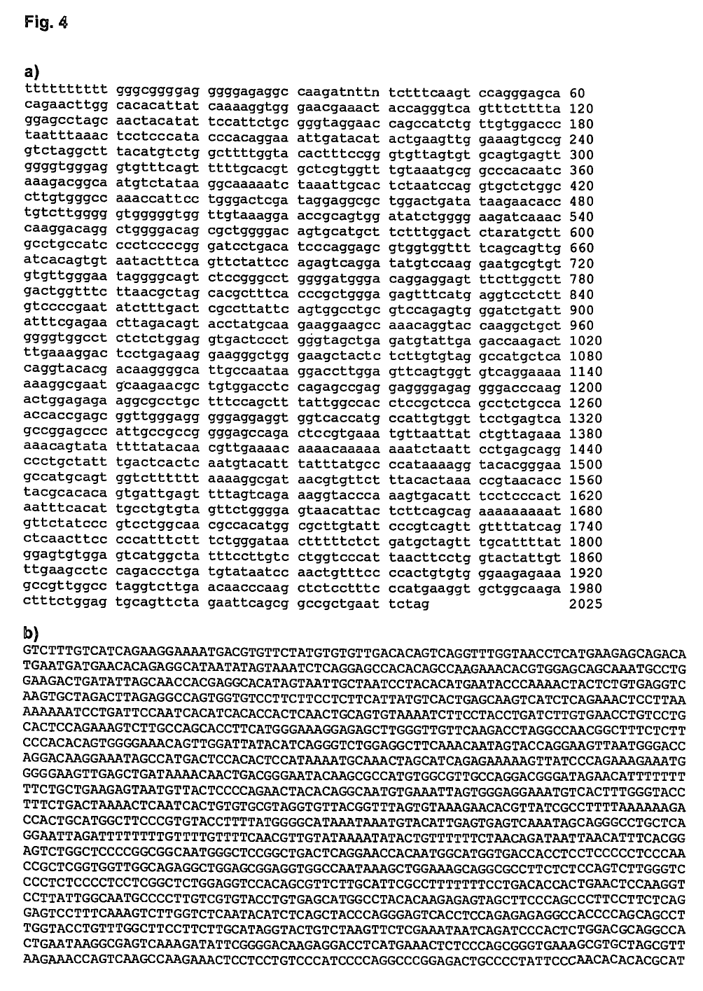

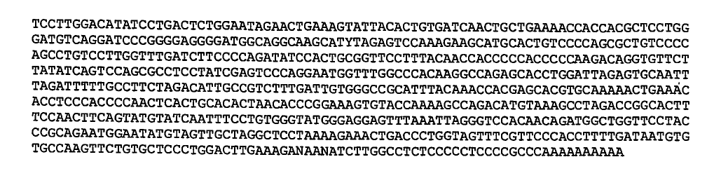

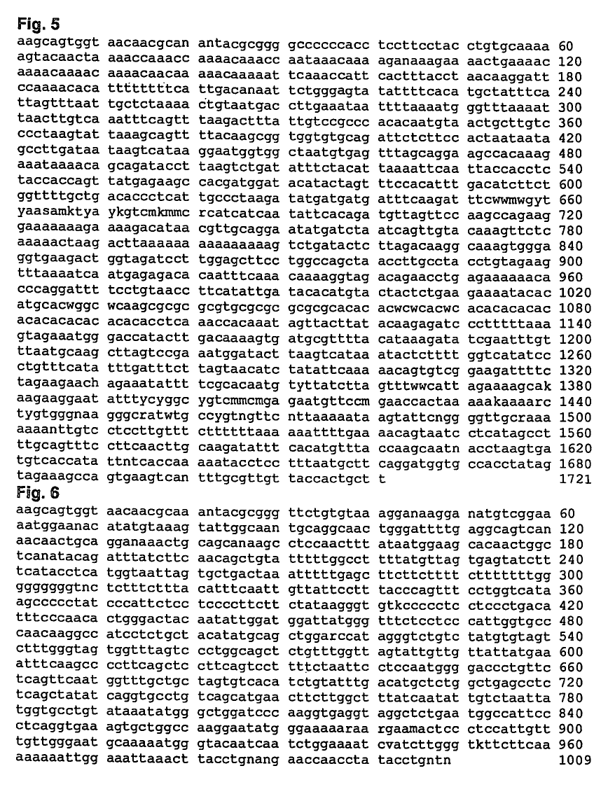

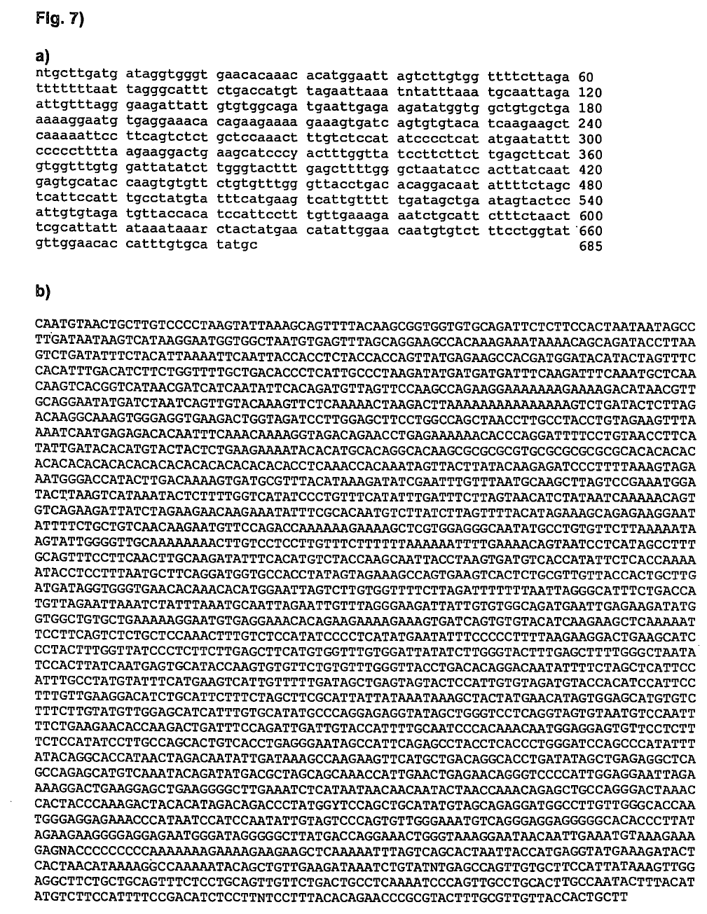

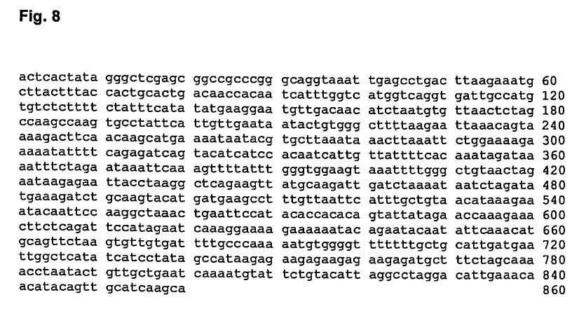

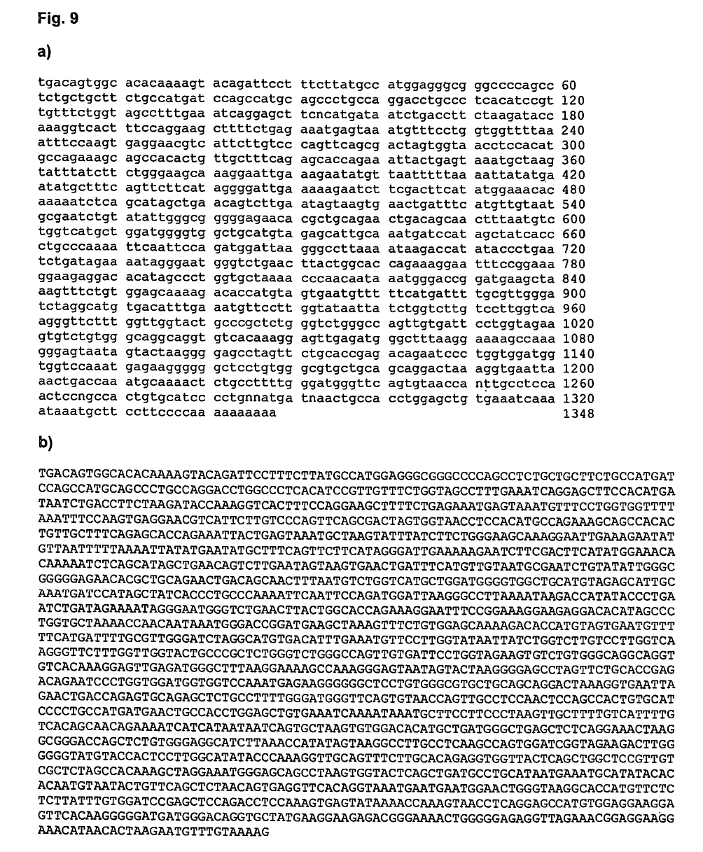

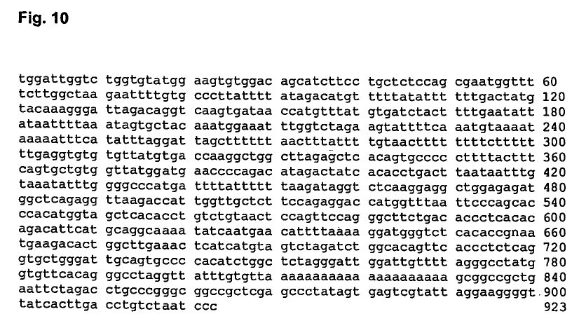

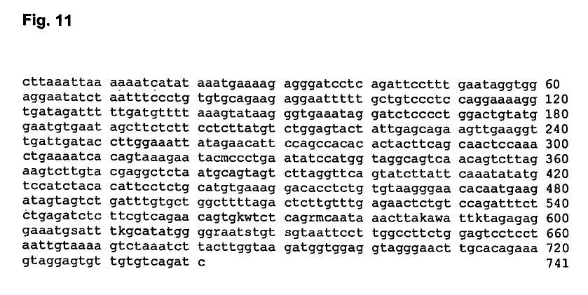

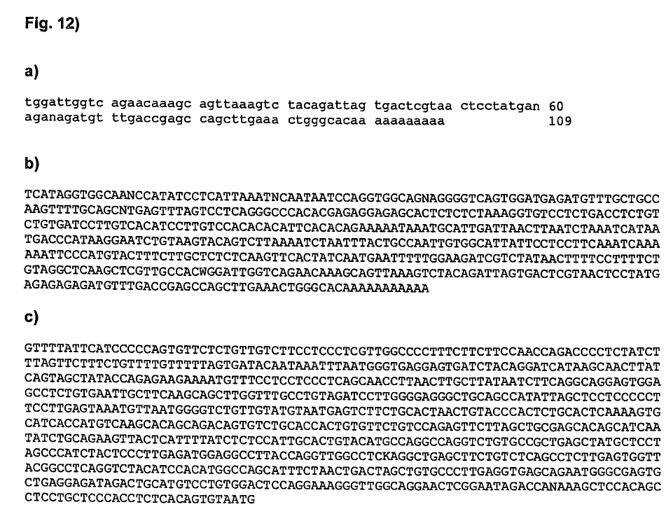

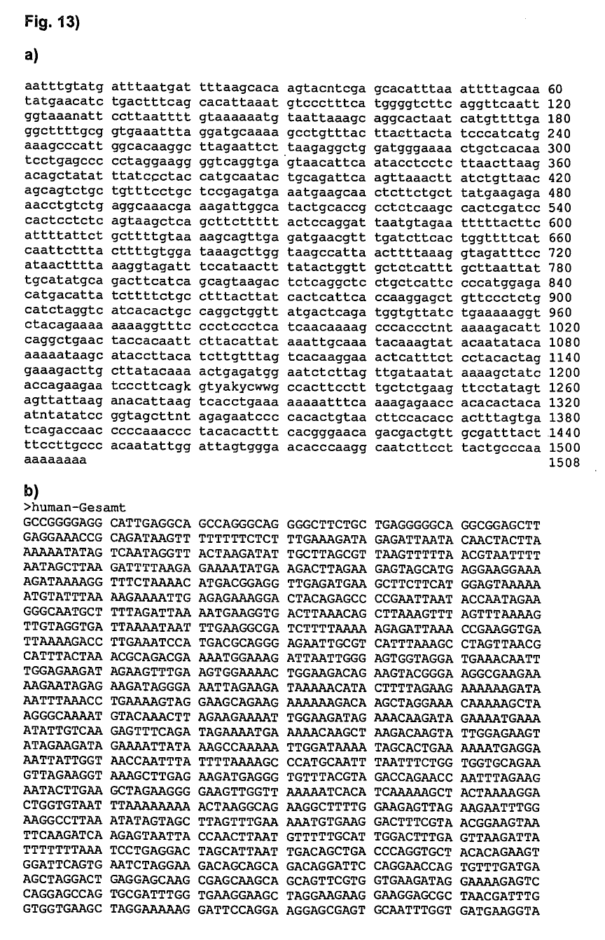

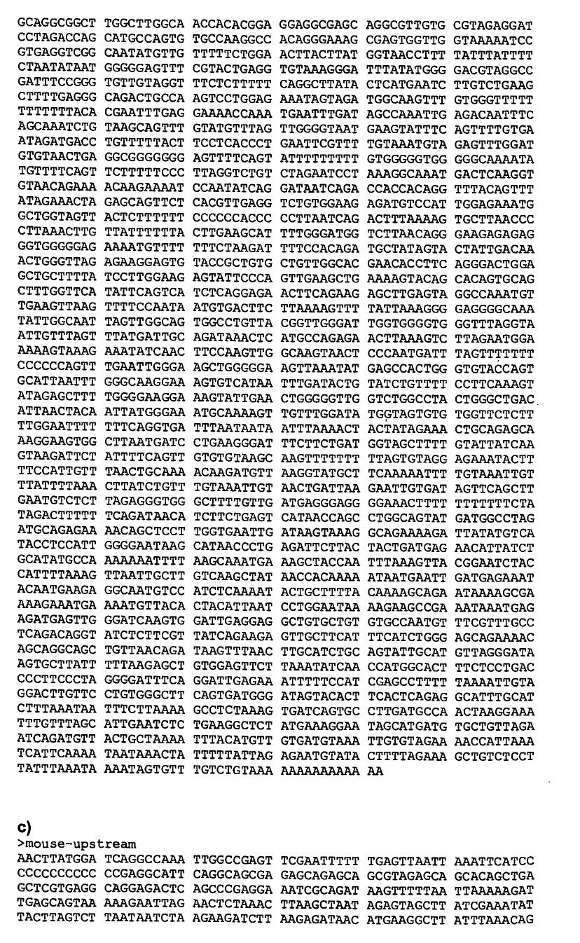

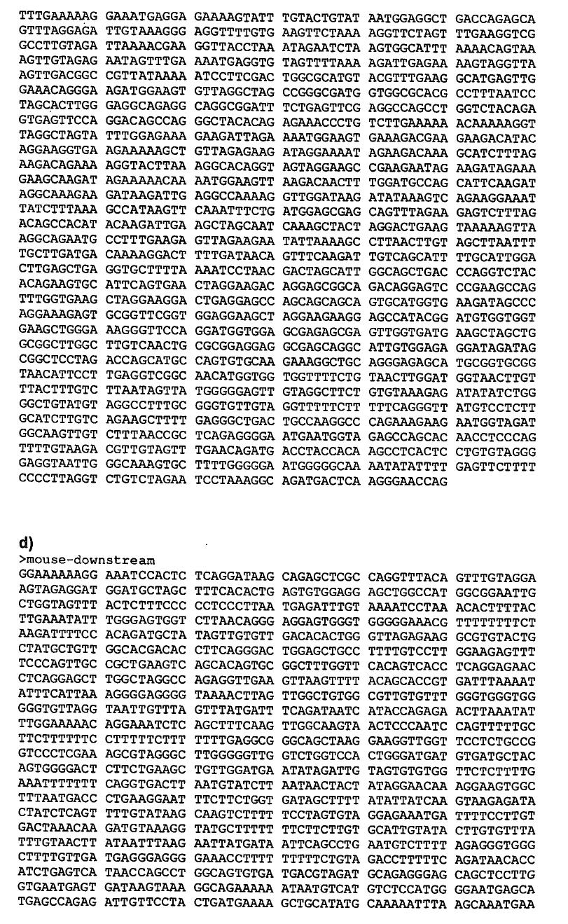

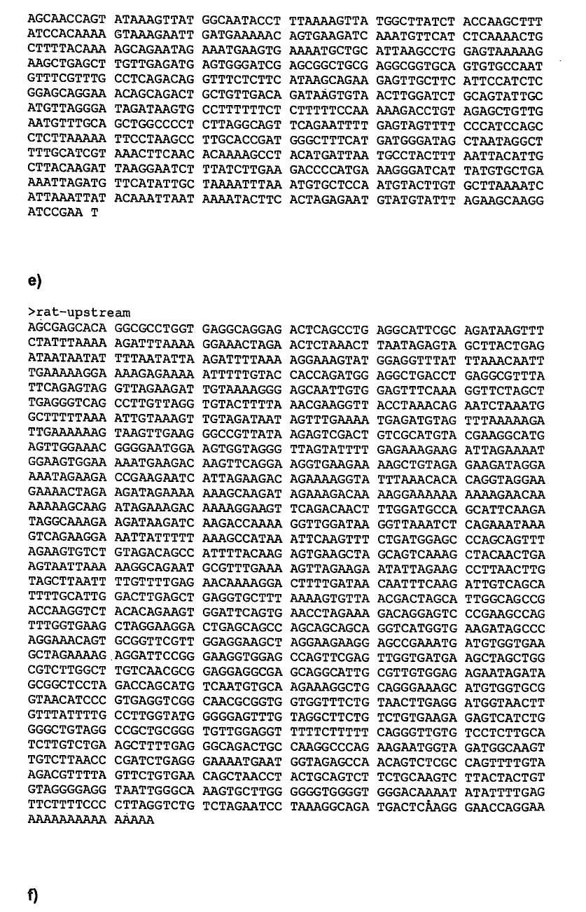

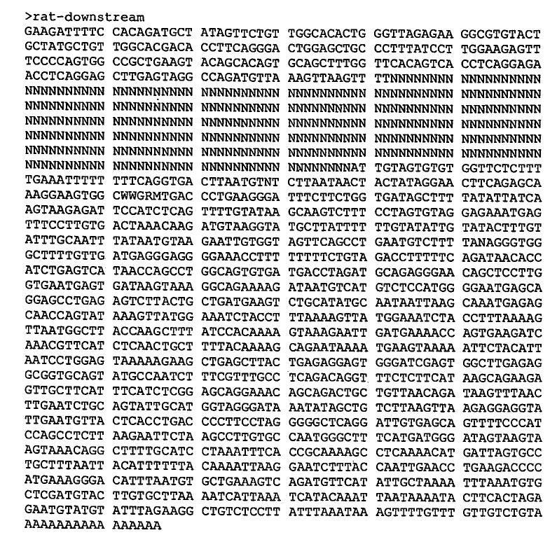

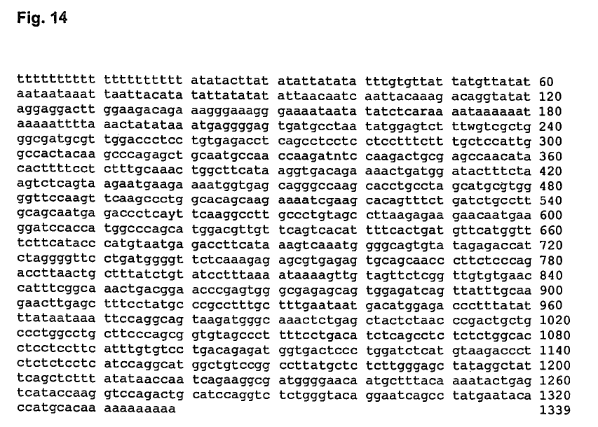

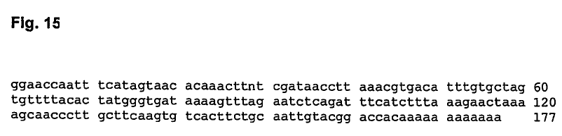

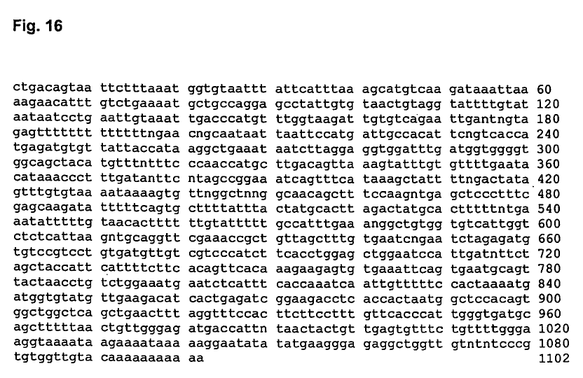

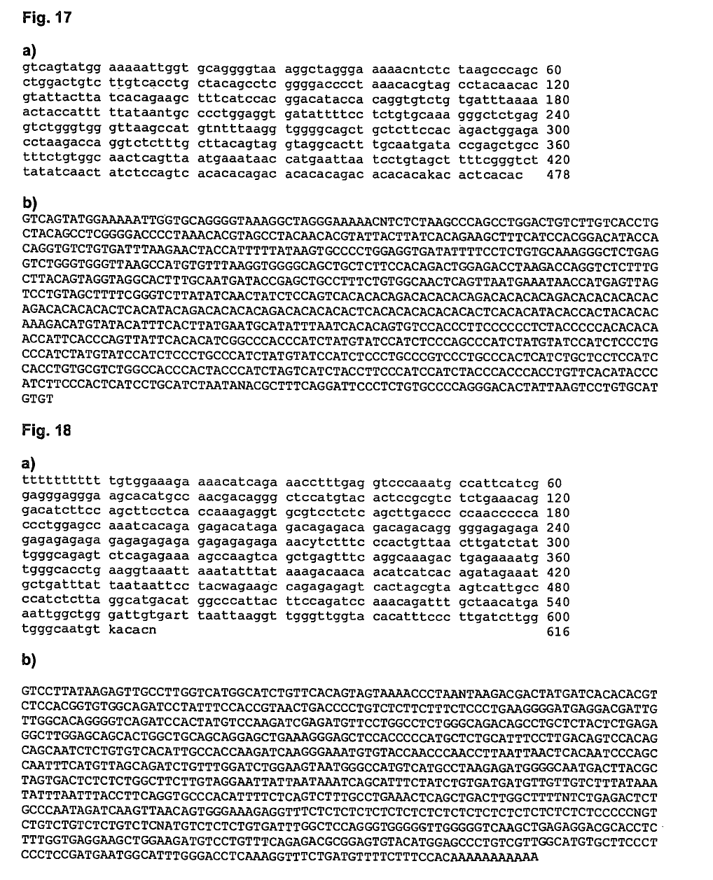

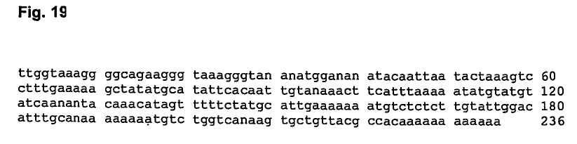

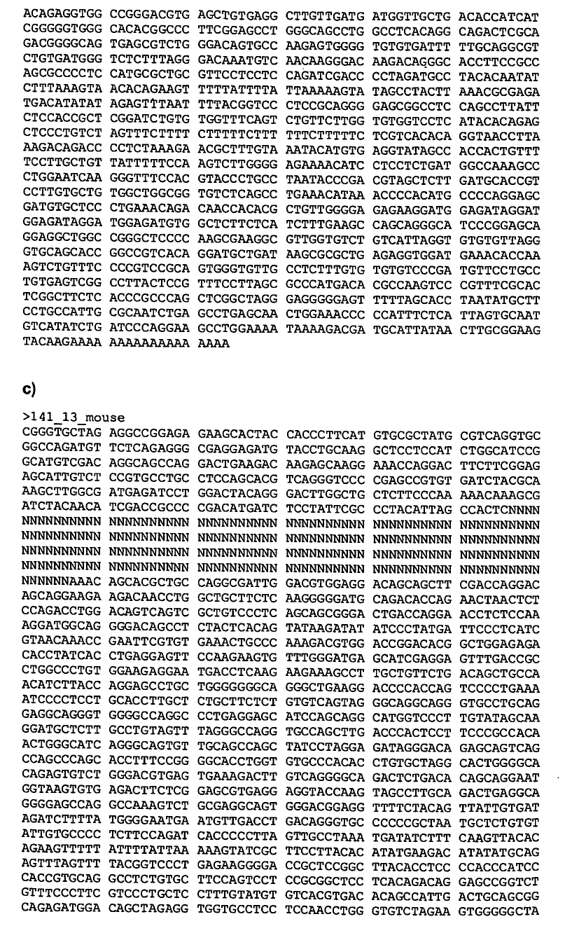

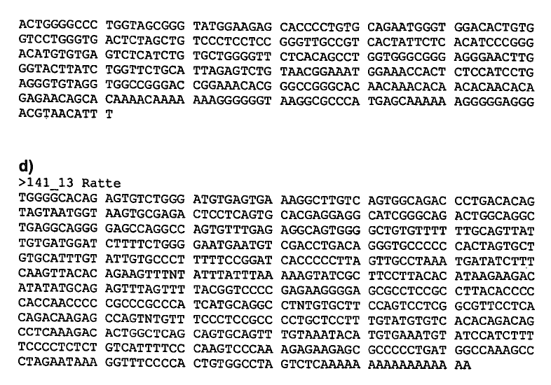

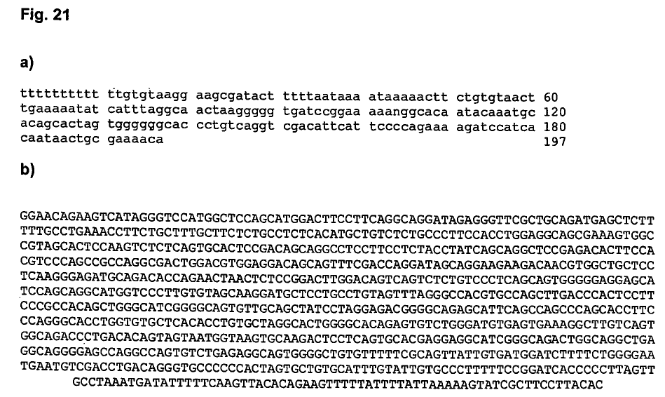

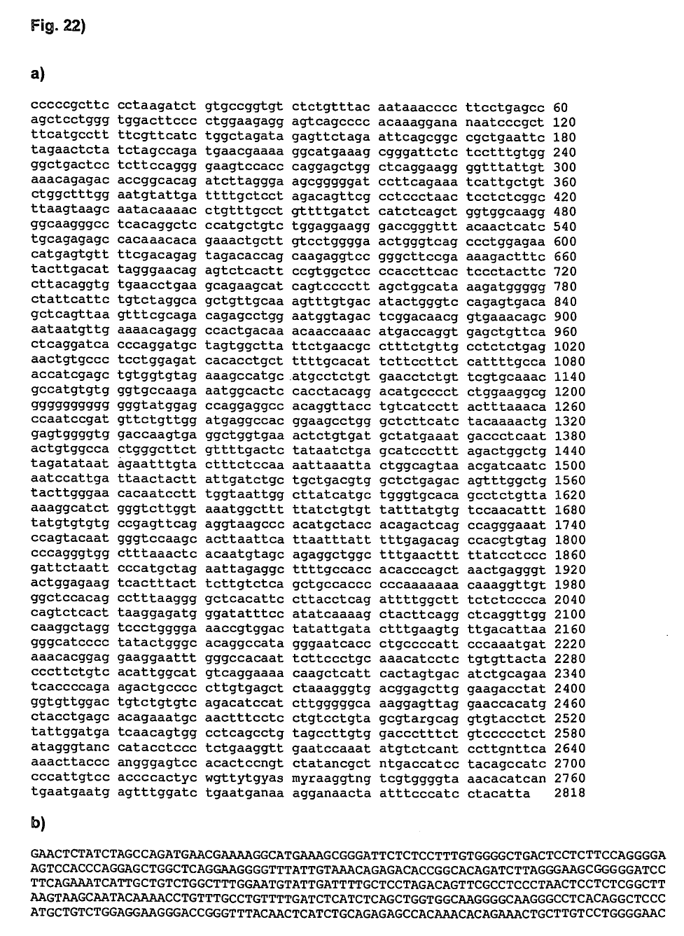

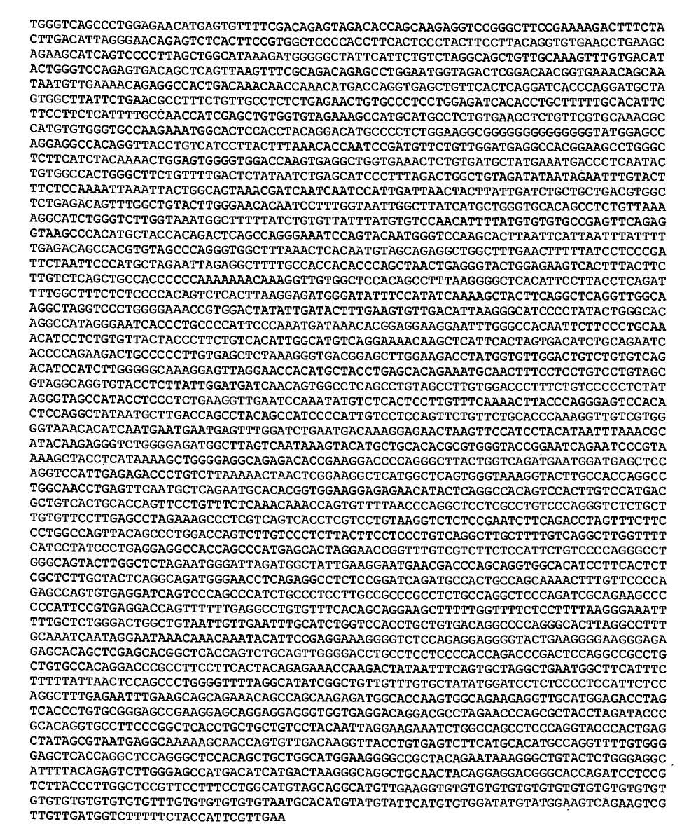

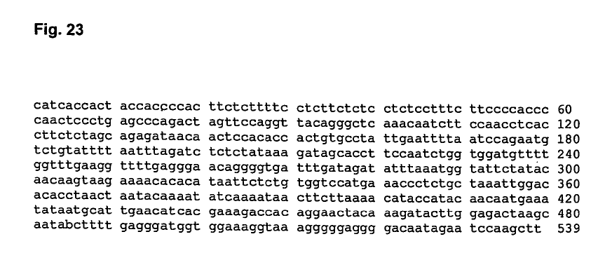

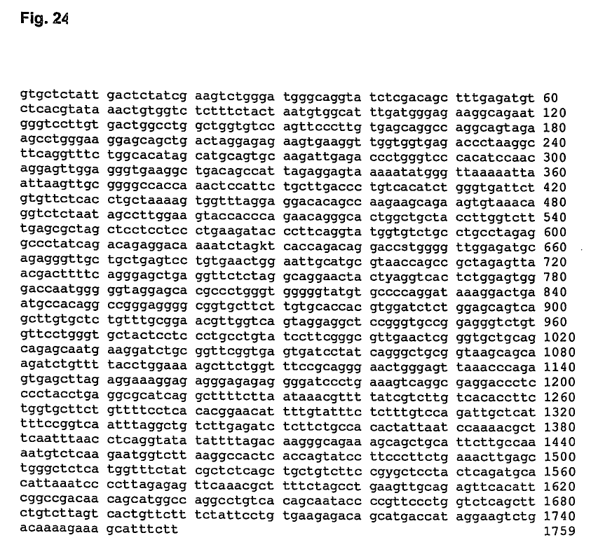

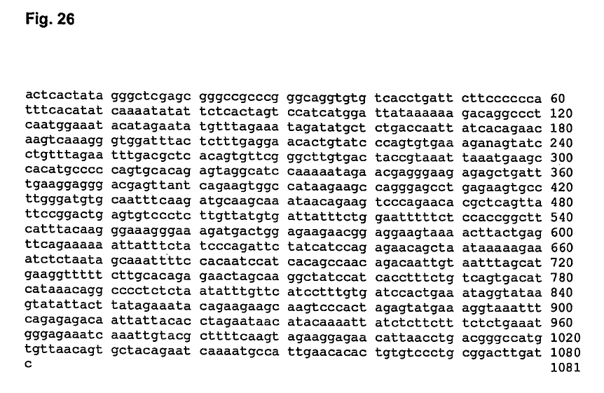

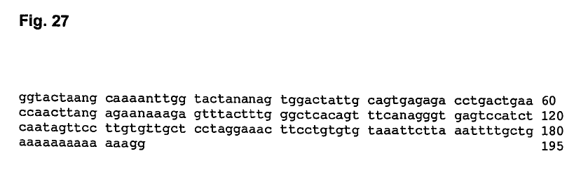

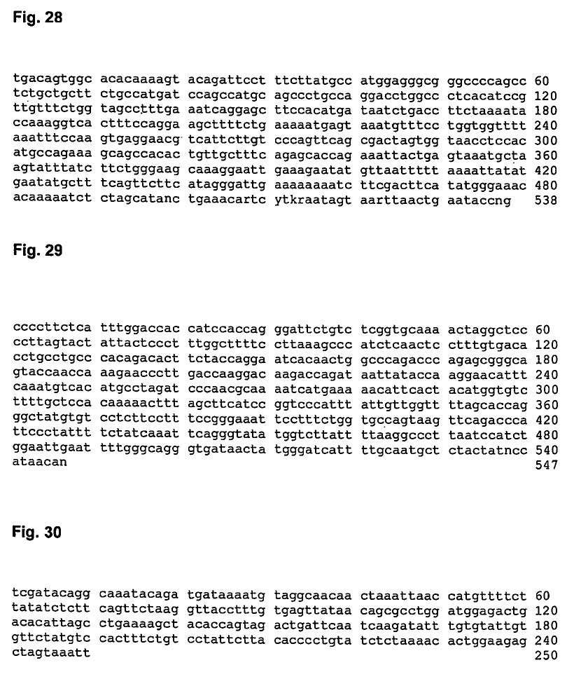

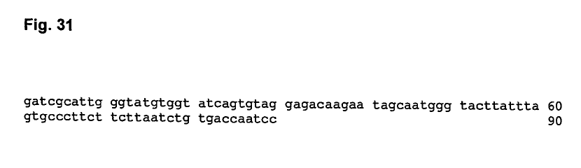

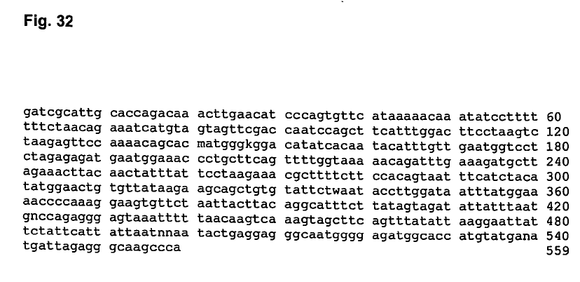

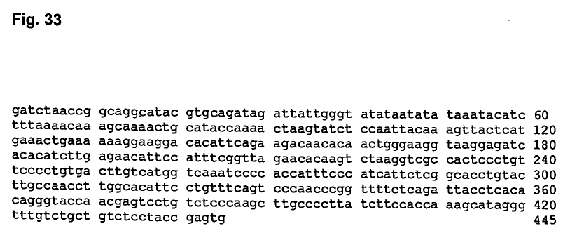

### Cloning of Full-length Human and Rat Sequences, or of the Corresponding Full-Length Gene for the Gene Fragments Found in Example 1 as per FIGS. 1-33

1.) Cloning of Full-length Rat cDNA-sequences

Cloning of full-length rat cDNA-sequences ensues either by screening through cDNA-libraries or by using PCR-based methods

a) Screening

The cDNA libraries were established using the RNA of a tissue that expresses the mRNA of the gene being searched for. Single- or double-stranded DNA or RNA-molecules, which are either radioactively labelled (e.g. with [α³²P] dCTP) or non-radioactively labelled (e.g. with digoxigenin-labelled UTP), can be used as probes. The cDNA library, provided in the form of bacteria or phages is placed on agar plates, is hybridised with the nucleic acid probes after transfer onto suitable filter membranes and determined after a stringent washing process. The cDNA clones identified by the above method are further isolated and analysed by means of sequencing (see Sambrooks et al., 1989; Ausubel et al., 1990).

b) PCR-based Method

In the PCR-based methods, oligonucleotide primers in sense or antisense orientation are determined and synthesised, using the regulated cDNA-sequences as a starting point. In the conventional 5′-RACE method (Rapid amplification of 5′-cDNA ends), the antisense-orientated primer is used for cDNA-synthesis, the resulting single-stranded cDNA is subsequently lengthened by ligation of an oligonucleotide with RNA-ligase or tailing-reaction with terminal transferase and then subjected to a PCR reaction with a primer located further in (known as a nested primer) and a tail-specific primer. The PCR amplificates are cloned and analysed by means of sequencing. One variant of said method utilises the so-called suppression PCR effect to extend the cDNA sequence both in the 5′ and in the 3′ direction, using the cDNA-template as a point of departure (Marathon cDNA Amplification Kit, Clontech). Here a double-stranded cDNA is obtained using as a point of departure RNA from rat spinal cord, and ligated with specially modified linkers. Said cDNA is then amplified by PCR using a gene-specific primer and a linker-specific primer, and the amplification products are cloned and sequenced.

2.) Cloning of Homologous Human cDNA-sequences

Homologous human cDNA sequences are isolated by modifying the above methods. In conventional screening of cDNA libraries, libraries of human origin are used and hybridisation is carried out under less stringent conditions in order to obtain cDNA-clones that are less homologous too.

PCR based strategies first attempt to amplify a human cDNA using a plurality of different primers which preferably originate from the encoded region. If this is not successful, then degenerated PCR-primers can be used or less stringent PCR conditions are selected.

### Example 3

### Carrying out the Screening Method By Measuring Binding Through the Suppression of a Radioactively Labelled Ligand

A nucleic acid segment which encodes the chloride channel CLC-7 is cloned in an expression vector that allows a constitutive expression (e.g. CMV-promoter) or an inducible expression in eukaryotic cells. The DNA is inserted into eukaryotic cells (e.g. CHO cells, HEK293-cells or NIH-3T3-cells) using a suitable transfection method, e.g. using Lipofectamin (Roche Diagnostics). The cells are cultured in the presence of a selection reagent (e.g. zeocin, hygromycin oder neomycin) such that the only cells that survive are those cells that have absorbed the DNA construct and, in cases where selection continues for a fairly long time, have incorporated said construct into the genome.

Using said cells as a point of departure, membrane fractions are obtained that contain the CLC-7 channel in a large amount and can be used for a binding assay. Said assay consists of 1.) the membrane containing the CLC-7; 2.) a radioactively labelled ligand (e.g. tritium-labelled 4,4′-diisothiocyanostilbene-2,2′-disulfonic acid (DIDS) or [³H]-labelled 4-acetamido-4′-isothiocyanatostilbene-2,2′-disulfonic acid (SITS)); 3.) a binding buffer (e.g. 50 mM HEPES pH 7.4, 1 mM EDTA) and the ligand to be studied for binding activity. In functional studies on CLC-7 expressing oocytes, the substances SITS and DIDS were capable of inhibiting CLC-7-mediated chloride channels (unpublished results: Dr. Diewald, University of Mainz). After incubation of the above reaction mixtures (for 30-60 min for example) at a suitable temperature (mostly room temperature), the non-bound radioactive ligand molecules were filtered off. The remaining quantity of bound [³H]-DIDS or [³H]-SITS is measured after adding a scintillation cocktail in a beta-counter (e.g. Trilux, Wallac). If the test substance shows binding to the CLC-7 channel, said substance is determined as a reduced radioactive insert. Said method is appropriately miniaturised, such that it can be carried out in (96-, 384-oder 1536-well) microtitre plates, in order to carry out said method using a robot in the so-called High throughput Screening (HTS) method.

### Example 4

### Carrying Out the Screening Method According to the Invention Using Measurement of the Functional Parameters Altered By the Binding of the Substance

A nucleic acid segment which encodes the protein kinase PIM-2 is cloned in an expression vector that allows an inducible expression in prokaryotes, such as, for example, E.coli. Here the nucleic acid segment is modified in such a way that it is expressed as a fusion protein having an additional N- or C-terminal amino acid sequence. The function of the PIM-2 kinase remaining unchanged, said sequence should allow purification using a specific method, e.g. glutathion S-transferase fragment, which allows isolation from the protein mixture by binding to glutathion. After transfection of the bacteria, induction of the gene (e.g. with IPTG in the lac-promoter) and solubilising of the bacteria, the fusion proteins are purified and used in an in vitro kinase experiment. Here 5 μg of protein are complemented at 30° C. for 30 minutes in 50 μl kinasebuffer (20 mM PIPES, pH 7.0, 5 mM MnCl₂, 7 mM β-mercaptoethanol, 0.4 mM spermine, 10 mMrATP) complemented with 10 μCi [γ³²P] ATP. Purified histone H1-Protein (Sigma) or bacterially expressed GST-NFATc1-fusion protein are added as substrates. After the incubation period, the non-incorporated [γ³²P] ATP is filtered off and the amount of incorporated ³²phosphate is determined by β scintillation (Trilux, Wallac). In an experiment to discover novel PIM-2 kinase-inhibitors, the test substances are co-incubated in said mixture and a decrease in the ³²P-incorporation is used as an indicator of an inhibitor. Said method is appropriately miniaturised, such that it can be carried out in (96-, 384-oder 1536-well) microtitre plates, in order to carry out said method using a robot in the so-called High Throughput Screening (FITS) method.

### Example 5

### Example of a Medicament Containing an Active Ingredient Tablet Formulation According to the Invention

Tablets can be produced by direct compression of mixtures of the active ingredient according to the invention with corresponding auxiliary substances or by compression of granules containing active ingredient (optionally with further auxiliary substances). In said method, the granules can be produced either by wet granulation using, for example, aqueous granulation liquids and subsequent drying of said granules or by dry granulation e.g. by compaction.

Direct compression

| | | | |
|---|---|---|---|
| e.g. per tablet: | 25 | mg | active ingredient according to the invention |
| | 271 | mg | Ludipress ™ (granules for direct tabletting |
| | | | from lactose monohydrate, Povidon K30 and |
| | | | Crospovidon) |
| | 4 | mg | magnesium stearate |
| | 300 | mg | in total |

Produce a homogeneous mixture of the active ingredient with the auxiliary substances and compress said mixture on a tabletting press into tablets having a Ø of 10 mm.

Dry granulation

| | | | |
|---|---|---|---|
| e.g. per tablet: | 25 | mg | active ingredient according to the invention |
| | 166 | mg | microcrystalline cellulose |
| | 80 | mg | low substituted hydroxypropyl cellulose (I- |
| | | | HPC LH |
| | | | 11 ™) |
| | 5 | mg | highly disperse silicon dioxide |
| | 4 | mg | magnesium stearate |
| | 280 | mg | in total |

Produce a homogeneous mixture of the active ingredient with the microcrystalline cellulose and the 1-HPC and compact said mixture. After sieving the compactates, the resulting granules are mixed with magnesium stearate and silicon dioxide and compressed on a tabletting press into tablets having a Ø of 9 mm.

Wet granulation

| | | | |
|---|---|---|---|
| e.g. per tablet: | 25 | mg | active ingredient according to the invention |
| | 205 | mg | microcrystalline cellulose |
| | 6 | mg | Povidon K30 |
| | 10 | mg | Crospovidon |
| | 4 | mg | magnesium stearate |
| | 250 | mg | in total |

Produce a homogeneous mixture of the active ingredient with the microcrystalline cellulose and the Crospovidon and granulate said mixture in a granulator with an aqueous solution of the Povidon. The wet granules are subsequently regranulated and after drying are dried for 10 h (at 50° C.) in a drying cabinet. The dry granules are sieved together with the magnesium stearate and finally mixed and compressed on a tabletting press into tablets having a Ø of 8 mm.

### Example 6

### Example of a Medicament Containing an Active Ingredient Tablet Formulation According to the Invention—Parenteral Solution

1 g of an active ingredient according to the invention is dissolved in 11 of water for injection purposes at room temperature and subsequently adjusted to isotonic conditions by adding NaCl (sodium chloride).

Bibliography:

Akopian A N, Sivilotti L & Wood J N (1995) Nature 379: 257-262

Ausubel F M, Brent R, Kingdton R E, Moore D D, Seidman J G, Smith J A & Struhl K eds. (1190) Current protocols in molecular biology. John Wiley & Sons, Inc. New York, N.Y.

Baba H. Doubell T P, Woolf C J 1999: Peripheral inflammation facilitates Aβ fiber-mediated synaptic input to the substantia gelatinosa of the adult rat spinal cord. J Neurosci 19: 859-867

Bauer D, Müller H, Reich J, Riedel H Ahrenkiel V, Warthoe P & Strauss M (1993): Identification of differentially expressed mRNA species by an improved display technique (DDRT-PCR) Nucl Acids Res 21: 4272-4280

Bonini A, Anderson S M, Steiner D F (1997) Molecular cloning and tissue expression of a novel orphan G Protein-coupled receptor from rat lung. Biochem Biophys Res Comm 234: 190-193

Chih-Cheng et al., (1995): A P2X prinoceptor expressed by a subset of sensory neurons. Nature 377: 428-432

Corderre T J, Katz J, Vaccarino A L, Melzack R (1993): Contribution of central plasticity to pathological pain: review of clinical and experimental evidence. Pain 52: 259-285

Dickenson (1995) Novel pharmacological targets in the treatment of pain. Pain Rev., 2, 1-12

Dubuisson et al., 1997 Pain, 4: 161-174

Feng Y & Gregor P (1997) Cloning of a novel member of the G Protein-coupled receptor family related to peptide receptors. Biochem Biophys Res Comm 231: 651-654

Furukawa T, Yang Y, Nakamoto B; Stamatoyannopoulos G, Papayannopoulou T (1996): Identification of new genes expressed in a human erythroleukemia cell line. Bloods Cell Mol & Dis 22: 11-22

Gunasekar P G, Kanthasamy, A G, Borowitz J L, Isom G E 1995: NMDA receptor activation produces concurrent generation of nitric oxide and reactive oxygen species: implication for cell death. J Neurochem 65: 20162021

Hawes B E, Fried S, Yao X, Weig B, Graziano M P 1998: Nociceptin (ORL1) and u-Opioid receptors mediate mitogen-activated protein kinase activation in CHO cells through a Gi-coupled signaling pathway: evidence for distinct mechanisms of agonist-mediated desensitization. J Neurochem 71: 10241033

Hubank M & Schatz D G (1994): Identifying differences in mRNA expression by representational difference analysis of cDNA Nucl Acids Res 22: 56405648

Klussmann S et al., 1996: Nature Biotechnology 14: 1112-1115

Li L-Y & Chang K-J 1996: The stimulatory effect of opioids on mitogenactivated protein kinase in chinese hamster ovary cells transfected to express u-opioid receptors. Mol Pharm 50: 599-602

Liang P & Pardee A B 1992: Differential Display of eukaryotic messenger RNA by means of the polymerase chain reaction. Science 257: 967-971

Methner A, Hermey G, Schinke B, Hermanns-Borgmeyer I (1997): A novel G Protein-coupled receptor with homology to neuropeptide and chemoattractant receptors expressed during bone development. Biochem Biophys Res Comm 233: 336-342

Mohit A A, Martin J H & Miller C A 1995: p493F12 Kinase: a novel MAP kinase expressed in a subset of neurons in the human nervous system. Neuron 14: 67-78

Poirier G M-C, Pyati J, Wan J S, Erlander M G 1997: Screening differentially expressed cDNA clones obtained by differential display using amplified RNA. Nucleic Acids Research 25: 913-914

Sambrook J, Fritsch E F & Maniatis T 1989: Molecular Cloning: A Laboratory Manual. Second Edition. Cold Spring Harbor Laboratory Press, Cold Spring Harbor

Sompayrac L, Jane S, Burn T C, Tenen D G & Danna K J 1995: Overcoming limitations of the mRNA differential display technique. Nucleic Acids Research 23: 4738-4739

Tal M 1996: A novel antioxidant alleviates heat hyperalgesia in rats with an experimental painful neuropathy. Neurreport 7: 1382-1384.

Tölle T R (1997): Chronischer Schmerz. In: Klinische Neurobiologie, Herdergen T, Tölle T R, Bahr M (Editor): p. 307-336; Spektrum Verlag, Heidelberg.

U.S. Pat. No. 5.262.3 11 Velculescu V E, Zhang L, Vogelstein B, Kinzler K W (1995): Serial analysis of gene expression. Science 270: 484-487.

Wan J S, Sharp J S et al. (1996): Cloning differentially expressed mRNAs Nature Biotech 14: 1685-1691.

Watson J B & Margulies J E (1993) Differential cDNA screening strategies to identify novel stage-specific proteins in the developing mammalian brain. Dev Neurosci 15: 77-86

Wilks A F (1989) Two putative protein-tyrosine kinases identified by application of the polymerase chain reaction. Poc Natl Acad Sci USA 86: 1603-1607

Woolf C J, Shortland P, Coggeshall R E 1992: Peripheral nerve injury triggers central sprouting of myelinated afferents. Nature 355: 75-78

Zimmermann, M & Herdegen, T (1996): Plasticity of the nervous system at the systemic, cellular and molecular levels: a mechanism of chronic pain and hyperalgesia. Progr Brain Res 110: 233-259

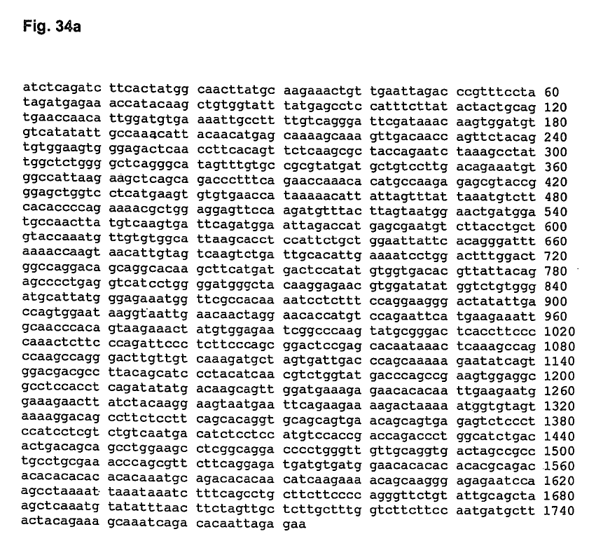

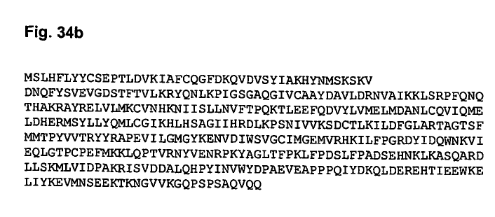

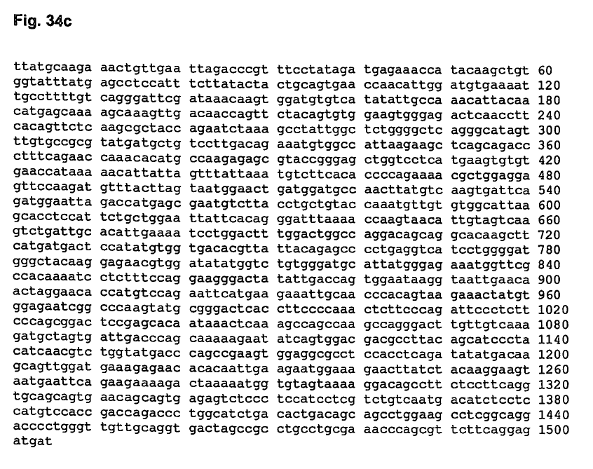

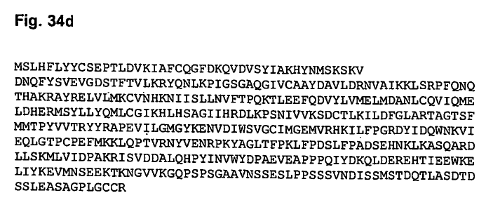

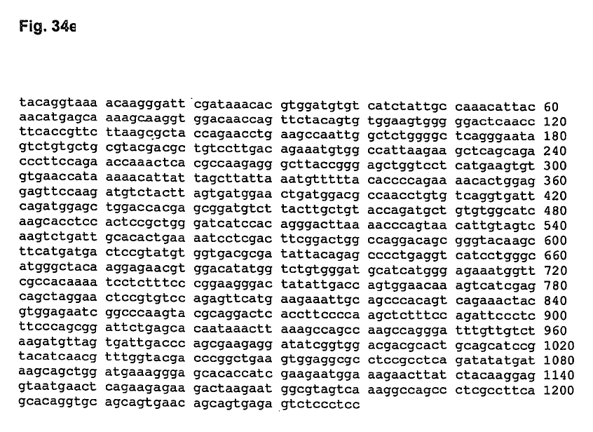

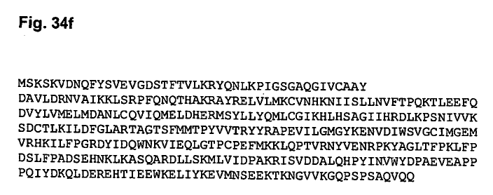

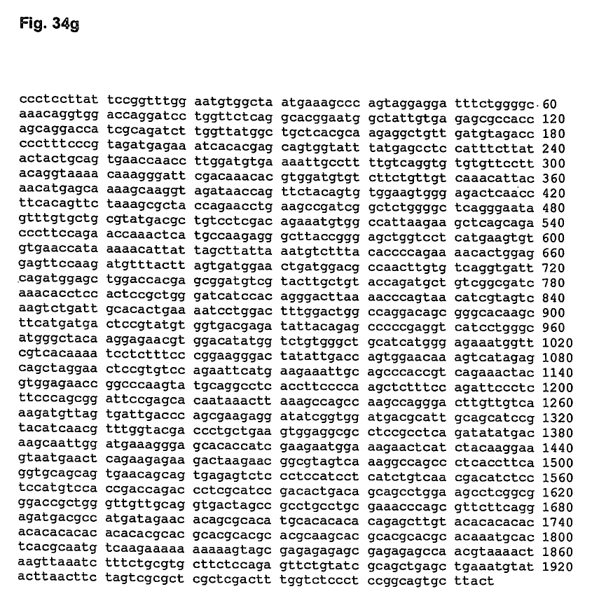

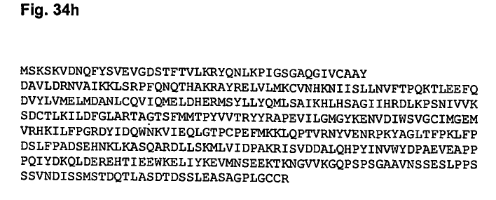

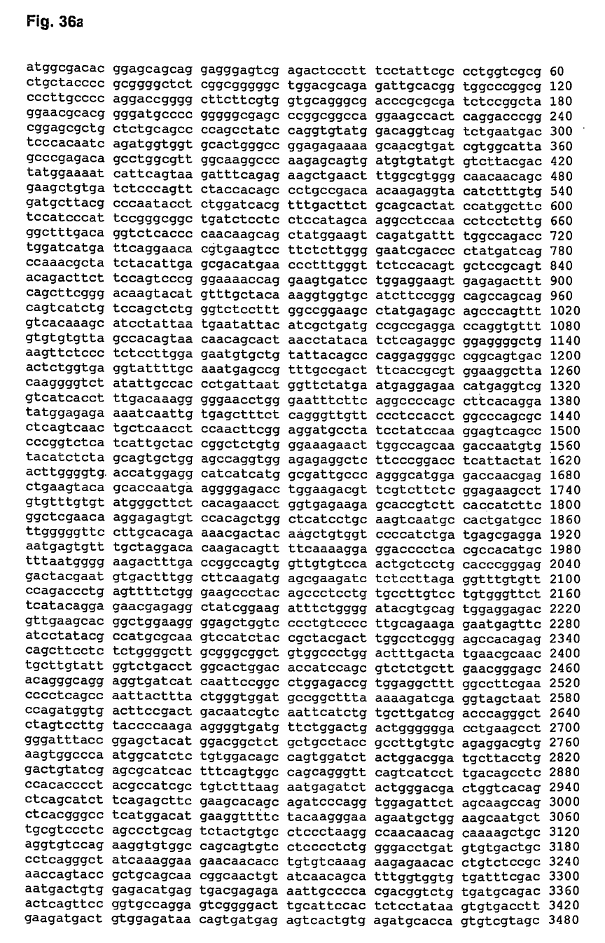

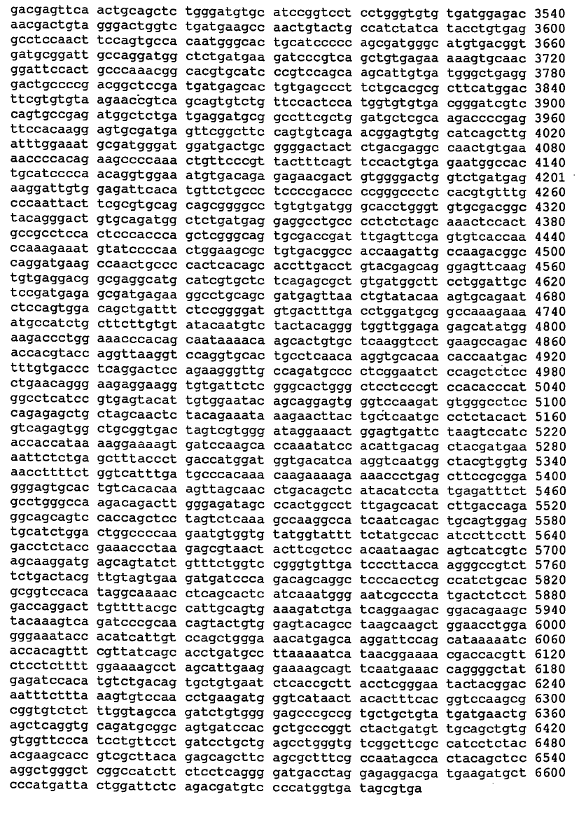

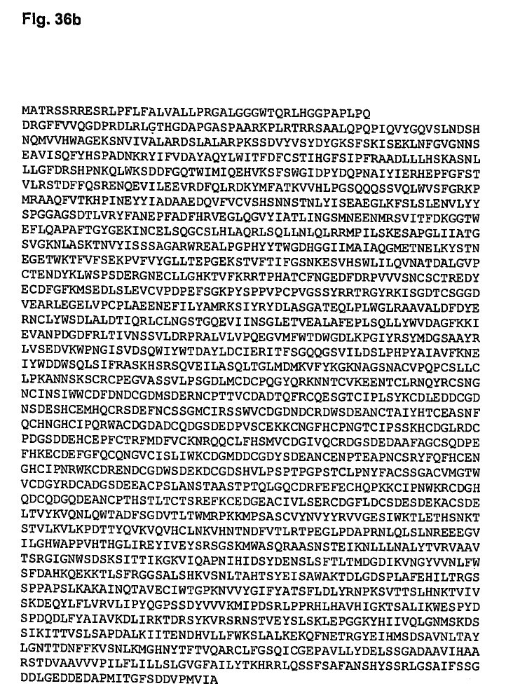

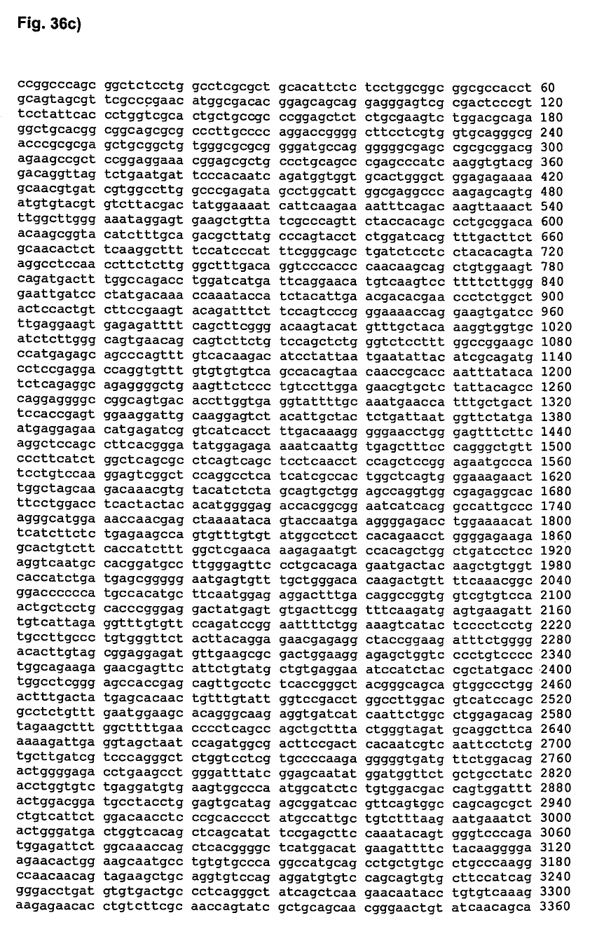

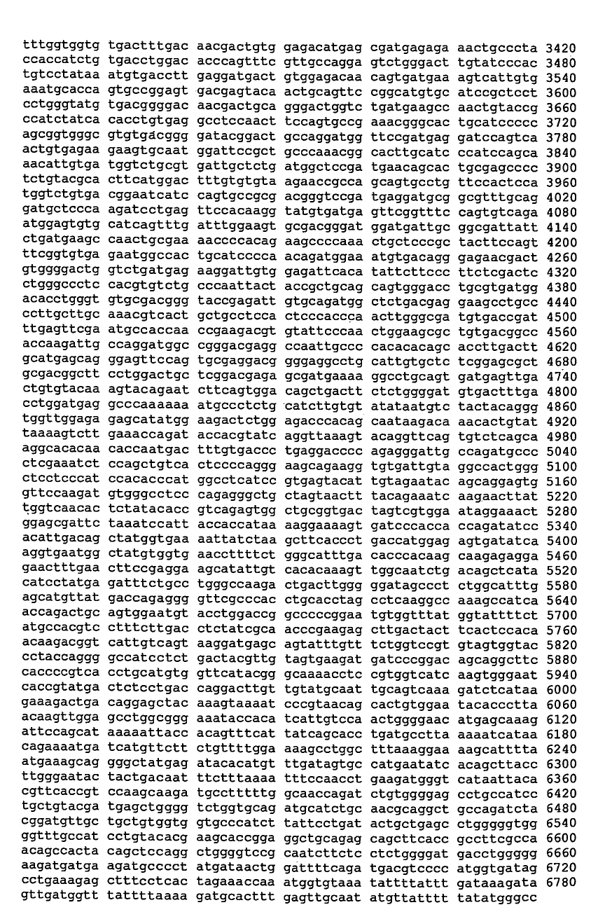

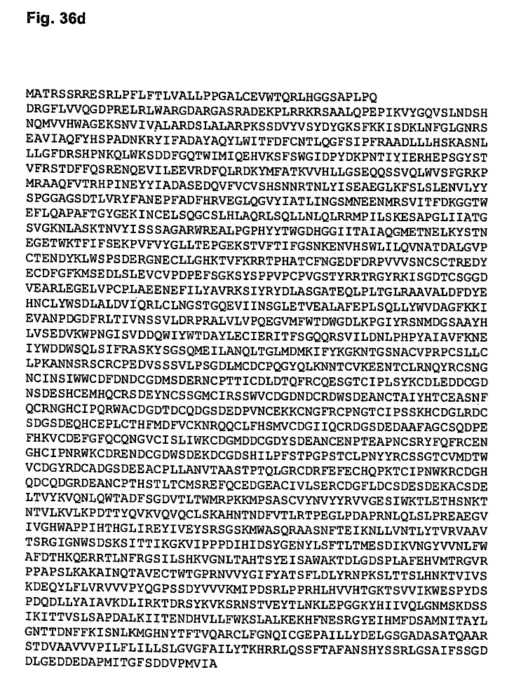

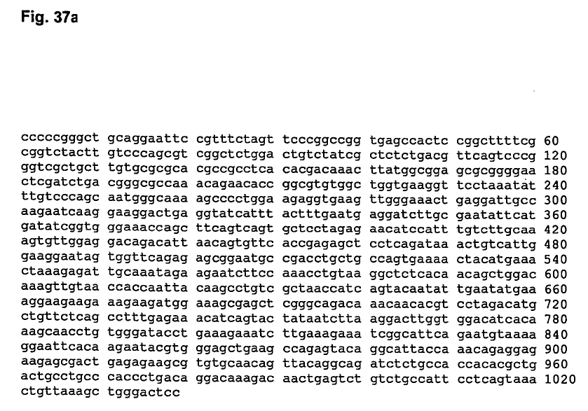

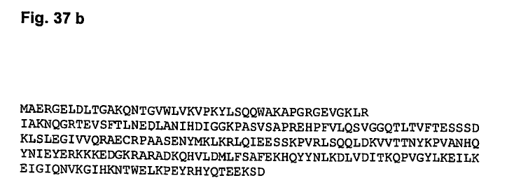

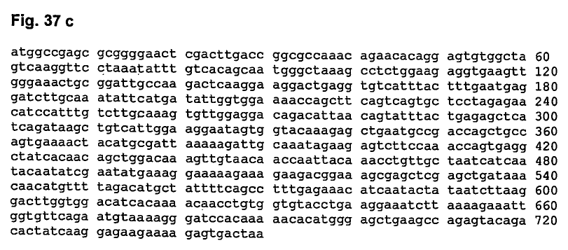

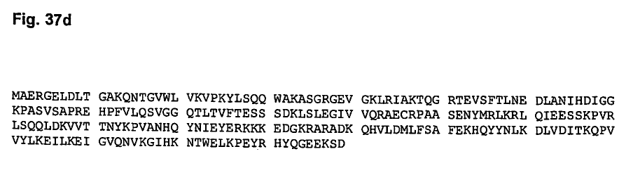

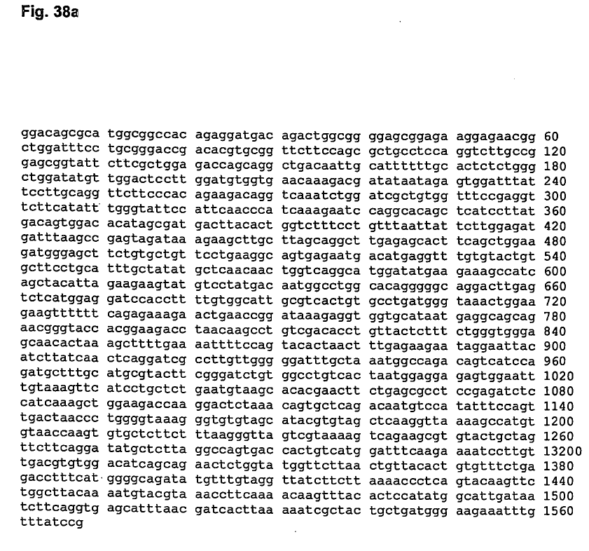

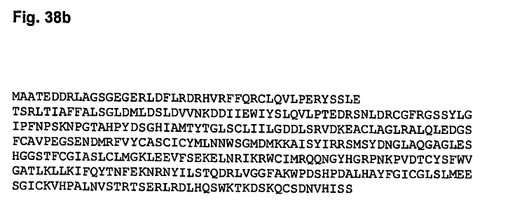

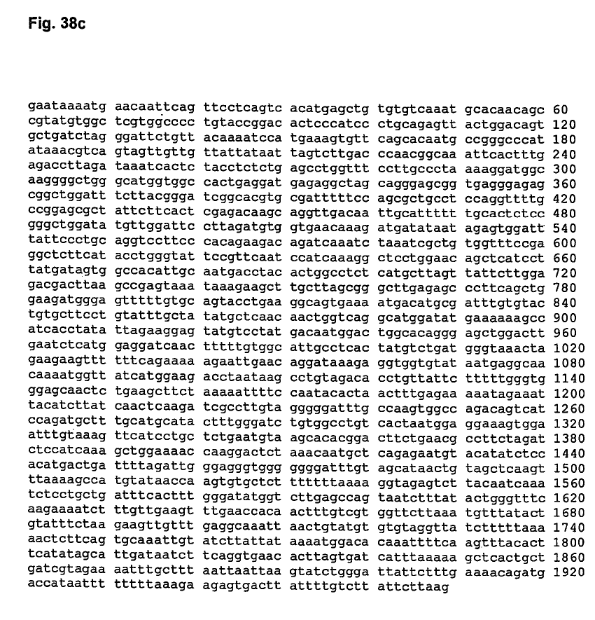

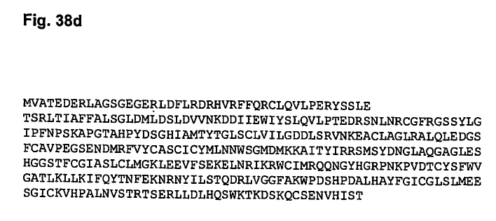

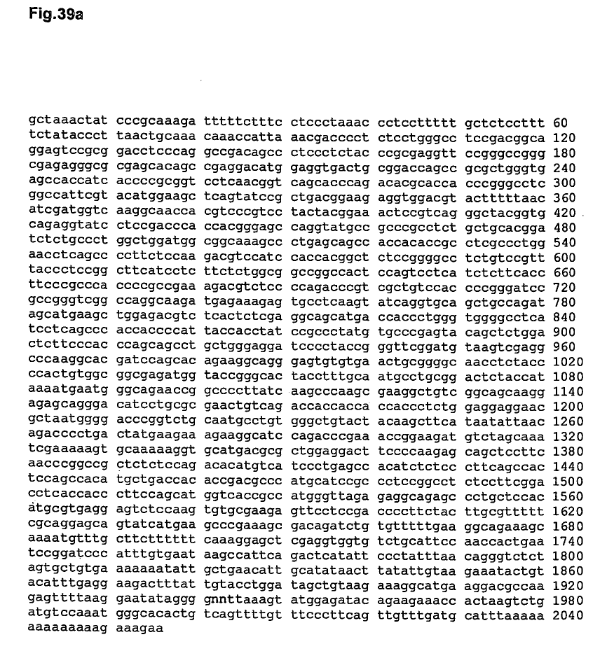

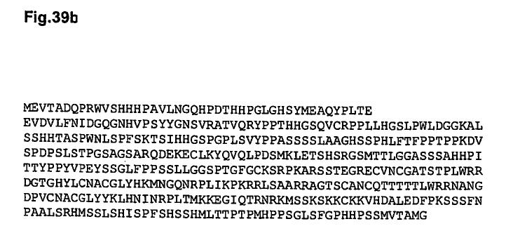

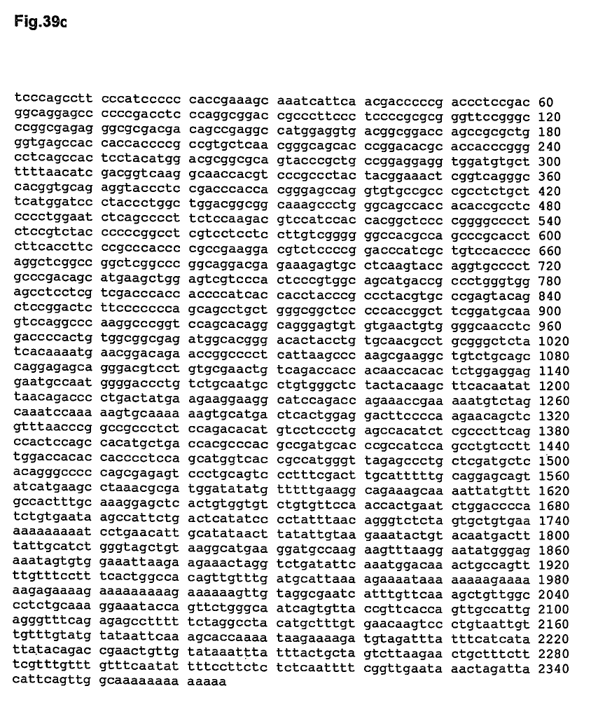

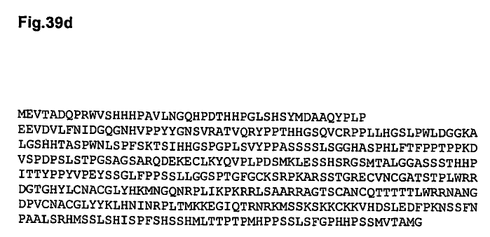

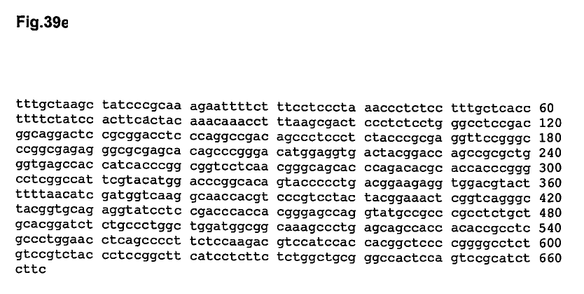

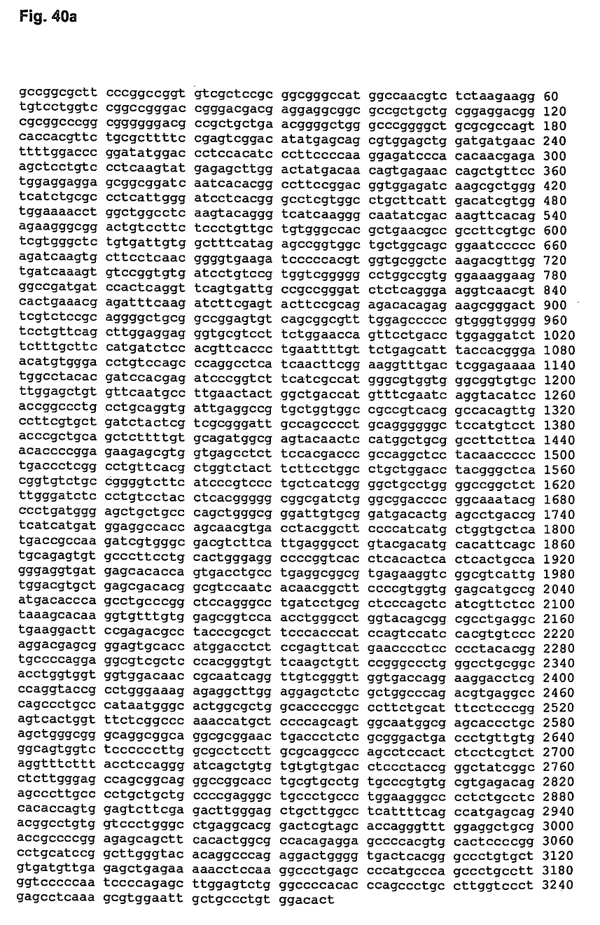

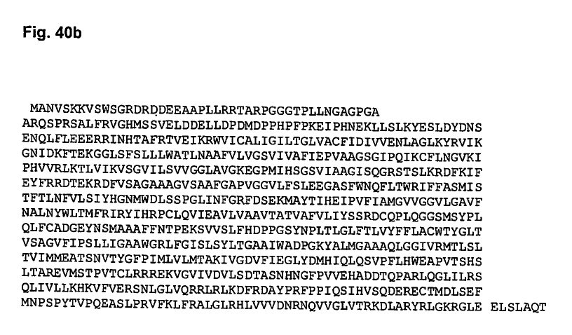

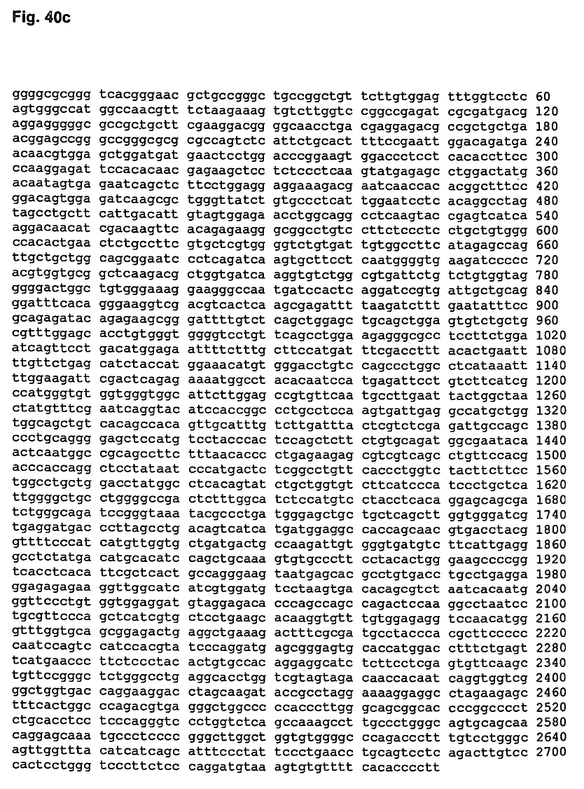

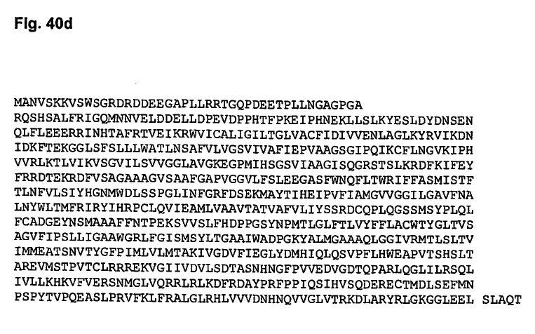

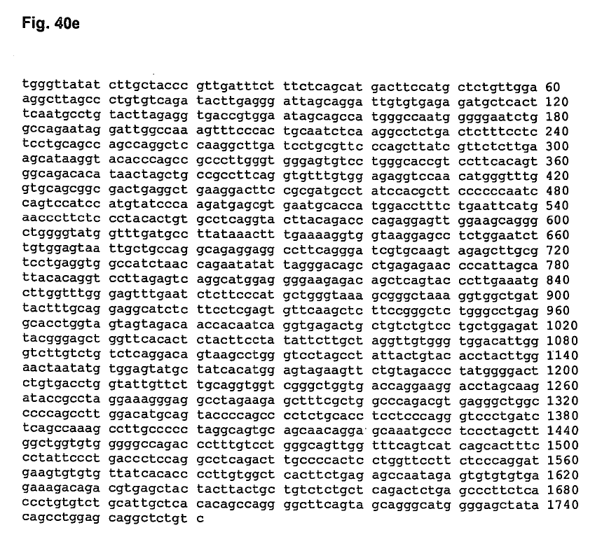

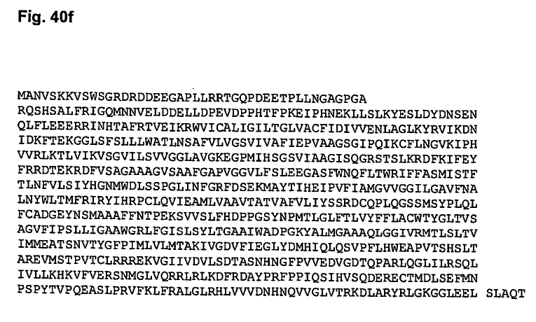

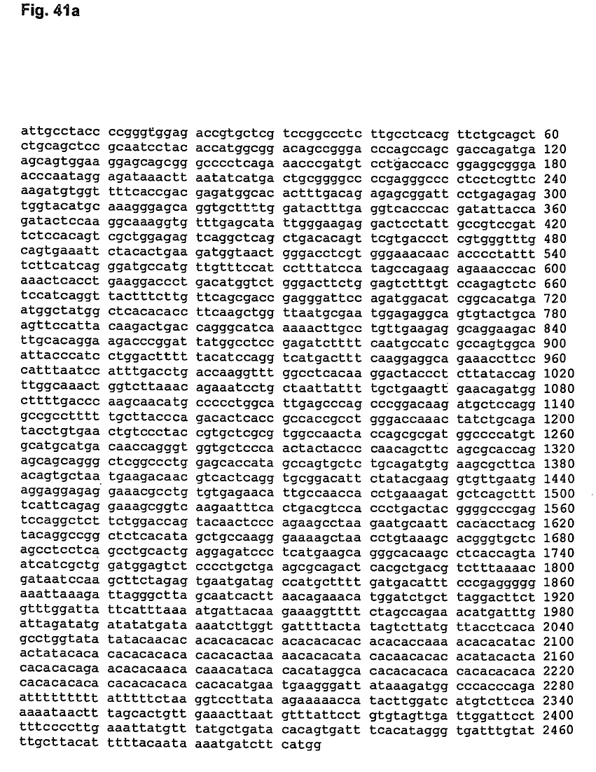

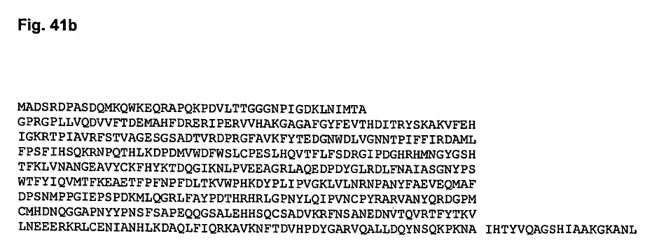

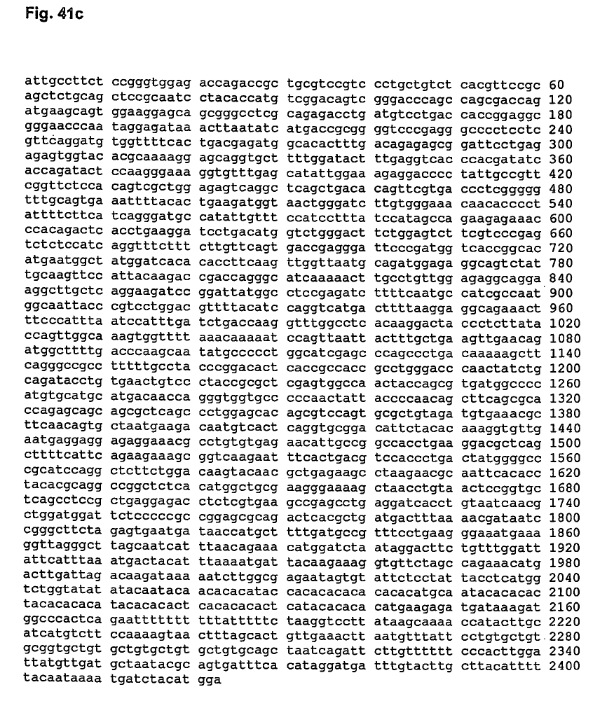

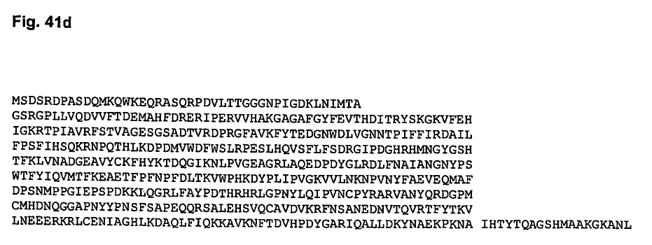

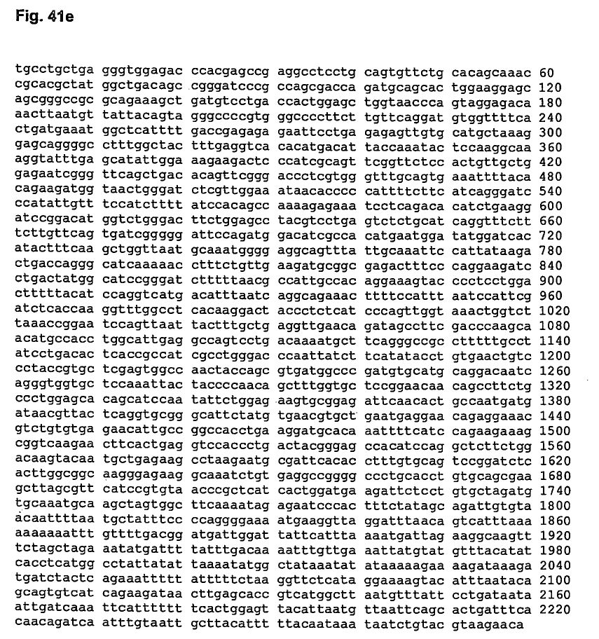

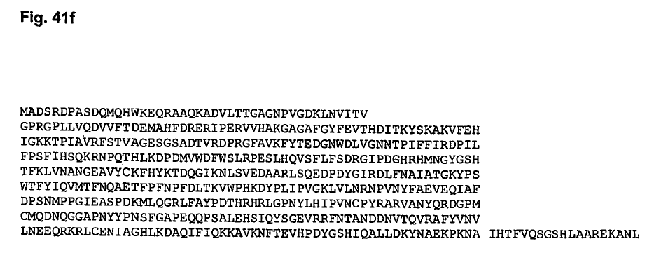

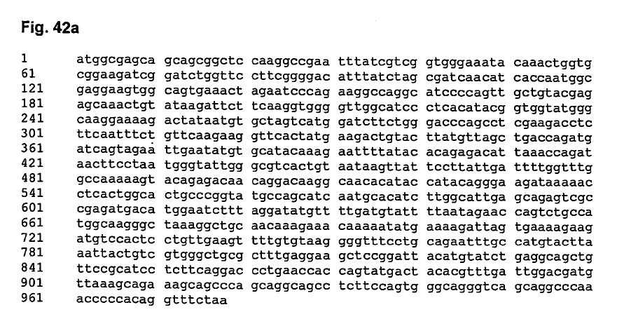

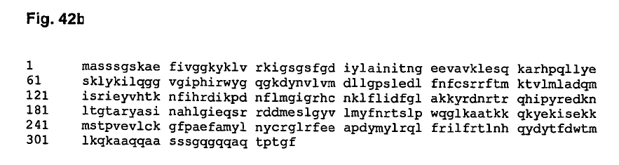

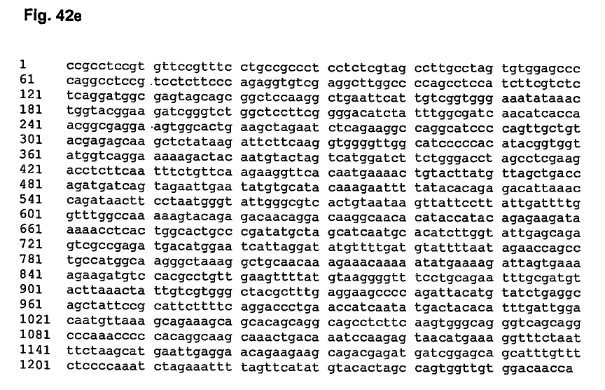

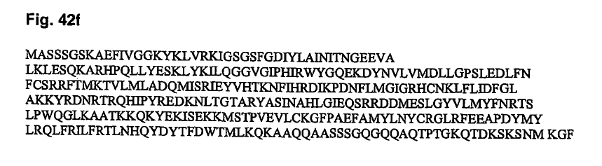

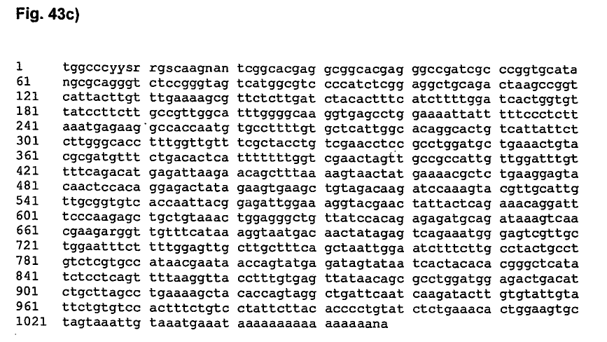

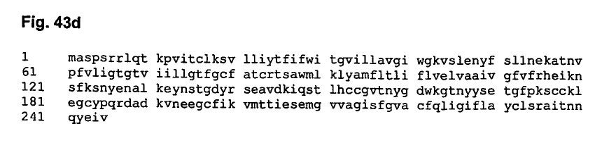

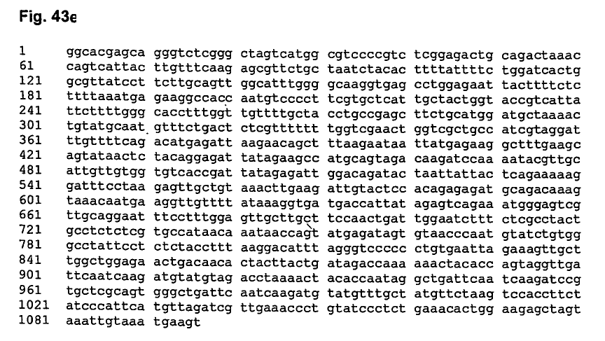

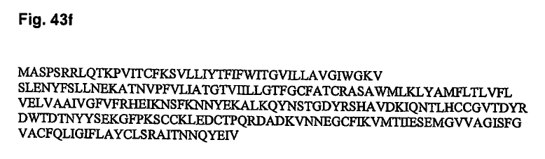

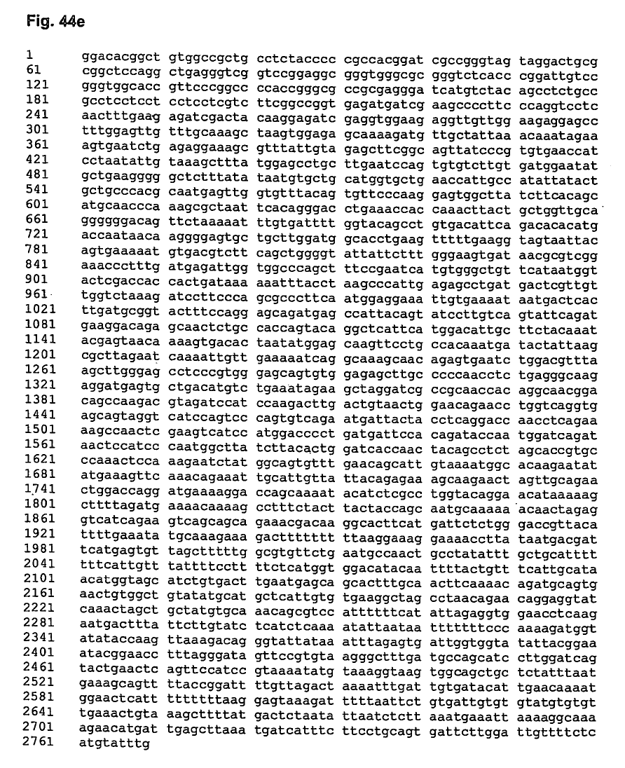

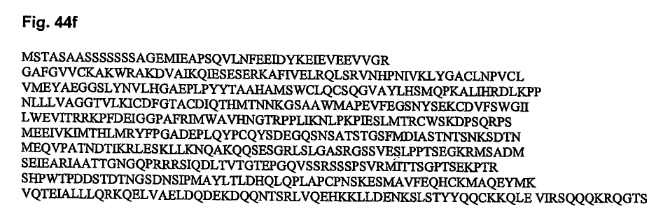

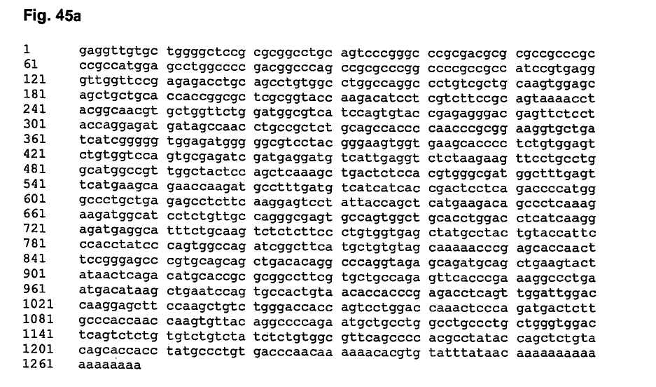

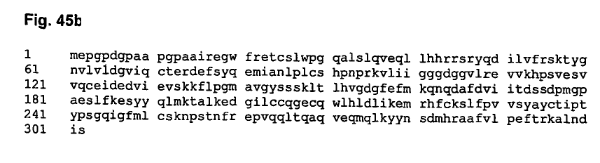

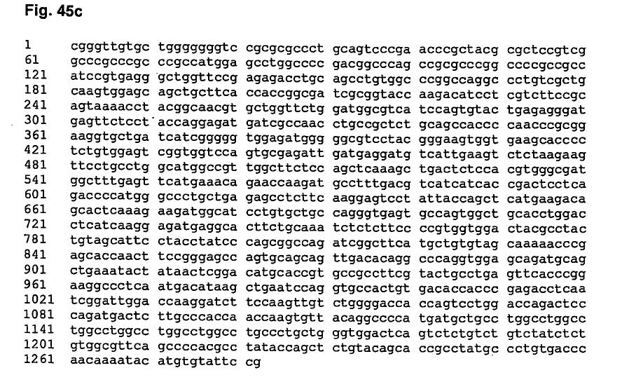

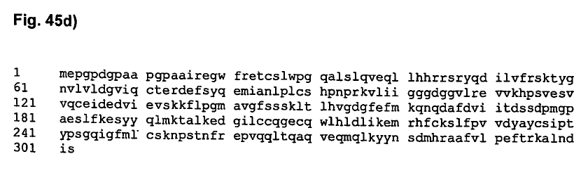

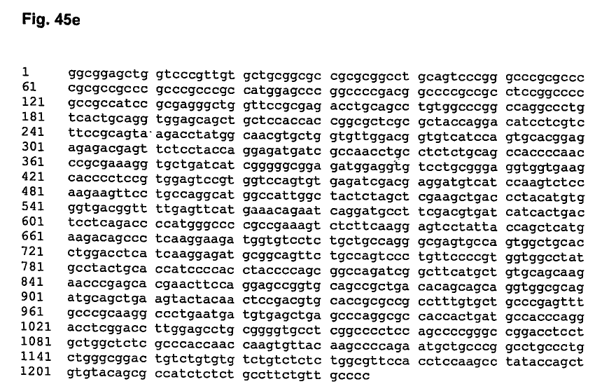

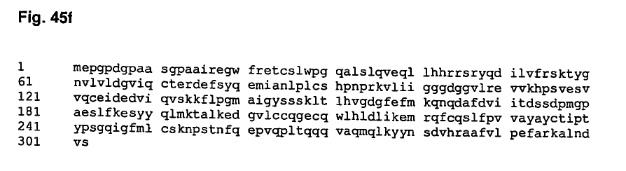

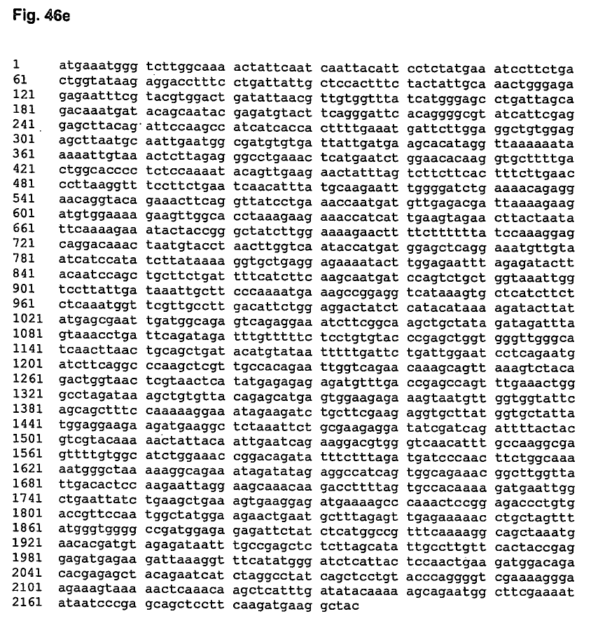

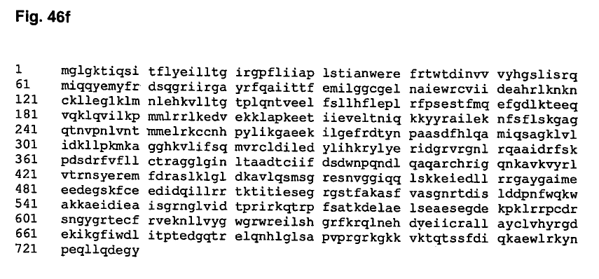

1. Method for discovering pain-regulating substances having the following process steps:
(a) incubation of a substance to be tested under appropriate conditions with a cell and/or a preparation from such a cell, which has synthesised a peptide or protein selected from the following group:
JNK3, PIM-2, LR-11, TFIIFβ, GGT-β, GATA3, CLC-7, catalase, tetraspanin (TM4SF/TSPAN-6/TM4-D), casein kinase 1a, MAP3K7/TAK-1 (TGF-β activated kinase), BAB14112 and/or spermidine synthase,
a peptide or protein which, or a part of which, is encoded by a polynucleotide according to any of FIGS. 34a), 34c), 34e), 34g), 35a), 35c), 35e), 36a), 36c), 37a), 37c), 38a), 38c), 39a), 39c), 39e), 40a), 40c), 40e), 41a), 41c), 41e), 42a), 42e), 43c), 43e), 44e), 45a), 45c), 45e), or 46e), or which is encoded by a polynucleotide which is at least 90% similar thereto,
a peptide or protein having an amino acid sequence according to any of FIGS. 34b), 34d), 34f), 34h), 35b), 35d), 35f), 36b), 36d), 37b), 37d), 38b), 38d), 39b), 39d), 40b), 40d), 40f), 41b), 41d), 41f), 42b), 42f), 43d), 43f), 44f), 45b), 45d), 45f) or 46f), or a peptide or protein at least 90% similar thereto
and/or a protein which is encoded by a nucleic acid which, under stringent conditions, binds to a polynucleotide according to any of FIGS. 34a), 34c), 34e), 34g), 35a), 35c), 35e), 36a), 36c), 37a), 37c), 38a), 38c), 39a), 39c), 39e), 40a), 40c), 40e), 41a), 41c), 41e), 42a), 42e), 43c), 43e), 44e), 45a), 45c), 45e), or 46e), or the antisense polynucleotide thereof,
a partial protein, at least 10 amino acids long, of any of the aforementioned proteins and/or peptides and/or
a peptide or protein which is encoded by a gene consisting of a polynucleotide, the nucleotide sequence whereof encompasses a gene fragment according to any of the sequences represented in FIGS. 1 to 33, or which is encoded by a polynucleotide at least 90% similar to such a gene,
(b) Measurement of the binding of the test substance to the peptide or protein synthesised by the cell or measurement of at least one functional parameter altered by the binding of the test substance to the peptide or protein.

2. Method according to claim 1, characterised in that the cell is genetically manipulated before step (a).

3. Method according to claim 2, characterised in that the genetic manipulation allows the measurement of at least one of the functional parameters altered by the test substance.

4. Method according to claim 3, characterised in that a form of a G protein not endogenously expressed in the cell is expressed as a result of the genetic manipulation, or in that a reporter gene is introduced.

5. Method according to any of claims 2 to 4, characterised in that the cell is genetically manipulated in such a way that the cell contains at least one polynucleotide according to any of FIGS. 34a), 34c), 34e), 34g), 35a), 35c), 35e), 36a), 36c), 37a), 37c), 38a), 38c), 39a), 39c), 39e), 40a), 40c), 40e), 41a), 41c), 41e), 42a), 42e), 43c), 43e), 44e), 45a), 45c), 45e), or 46e) or a polynucleotide at least 90% similar thereto, or a polynucleotide from the coding sequence of a gene which encompasses a gene fragment according to any of the sequences represented in FIGS. 1 to 33, or a polynucleotide at least 90% similar thereto.

6. Method according to claim 5, characterised in that the polynucleotide is contained in a recombinant DNA construct.

7. Method according to any of claims 2 to 6, characterised in that after the genetic manipulation according to claim 2 and before step (a) according to claim 1, the cell is cultivated under conditions which allow expression, optionally under selection pressure.

8. Method according to any of claims 1 to 7, characterised in that the cell is an amphibian cell, a bacterial cell, a yeast cell, an insect cell or an immortalised or native mammalian cell.

9. Method according to any of claims 1 to 8, characterised in that measurement of the binding takes place via the suppression of a known labelled ligand of the peptide or protein according to claim 1 and/or via the activity bound thereto of a labelled test substance

10. Method according to any of claims 1 to 8, characterised in that the measurement of at least one of the functional parameters altered by the test substance takes places by means of measuring the regulation, inhibition and/or activation of receptors, ion channels and/or enzymes, in particular by means of measuring the alteration in gene expression, the ion concentration, the pH or the membrane potential via change in the enzyme activity or the concentration of the 2^{nd} messenger.

11. Method according to any of claims 1 to 10, characterised in that the peptide or protein in steps (a) and (b) is selected from the following groups:
JNK3, PIM-2, LR-11, TFIIFβ, GGT-β, GATA3, CLC-7, catalase, tetraspanin (TM4SF/TSPAN-6/TM4-D), casein kinase 1a , MAP3K7/TAK-1 (TGF-β activated kinase), BAB14112 and/or spermidine synthase,
a peptide or protein which, or a part of which, is encoded by a polynucleotide according to any of FIGS. 34a), 34c), 34e), 34g), 35a), 35c), 35e), 36a), 36c), 37a), 37c), 38a), 38c), 39a), 39c), 39e), 40a), 40c), 40e), 41a), 41c), 41e), 42a), 42e), 43c), 43e), 44e), 45a), 45c), 45e), or 46e), or which is encoded by a polynucleotide which is at least 90%, preferably 95%, in particular 97% similar thereto or
a peptide or protein having an amino acid sequence according to any of FIGS. 34b), 34d), 34f), 34h), 35b), 35d), 35f), 36b), 36d, 37b), 37d), 38b), 38d), 39b), 39d), 40b), 40d), 40f), 41b), 41d), 41f), 42b), 42f), 43d), 43f), 44f), 45b), 45d), 45f) or 46f) or a peptide or protein at least 90%, preferably 95%, in particular 97% similar thereto,
and/or a protein which is encoded by a nucleic acid which, under stringent conditions, binds to a polynucleotide according to any of FIGS. 34a), 34c), 34e), 34g), 35a), 35c), 35e), 36a), 36c), 37a), 37c), 38a), 38c), 39a), 39c), 39e), 40a), 40c), 40e), 41a), 41c), 41e), 42a), 42e), 43c), 43e), 44e), 45a), 45c), 45e), or 46e), or the antisense polynucleotide thereof and/or
a partial protein at least 10, preferably 15, in particular at least 20 amino acids long, of any of the aforementioned proteins and/or peptides.

12. Method according to any of claims 1 to 10, characterised in that a gene consisting of a polynucleotide which encompasses a gene fragment according to any of the sequences represented in FIGS. 1, 3-8, 10, 11, 13, 14, 15, 17-27 or 31-33, or a polynucleotide at least 90%, in particular 95%, preferably 97% similar to said gene, encodes the peptide or protein in steps (a) and (b).

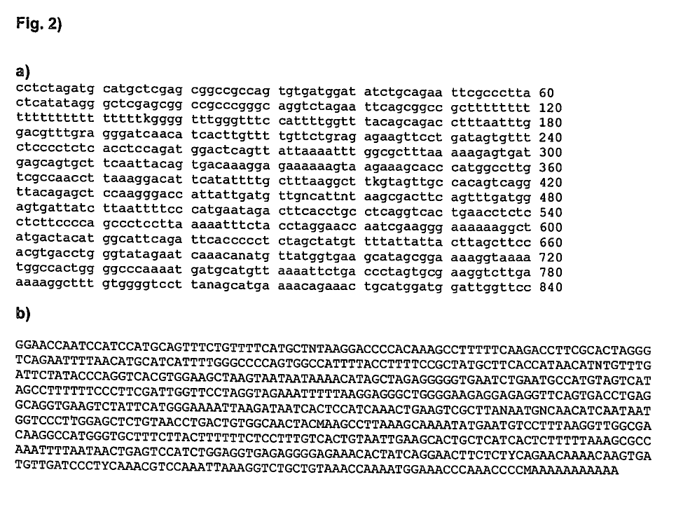

13. Method according to any of claims 1 to 10, characterised in that a gene consisting of a polynucleotide which encompasses a gene fragment according to any of the sequences represented in FIGS. 2, 9, 12, 16 or 28-30, or a polynucleotide at least 90%, in particular 95%, preferably 97% similar to said gene, encodes the peptide or protein in steps (a) and (b).

14. Polynucleotide which corresponds by at least 90%, preferably 95%, in particular at least 97% to any of the nucleotide sequences represented in any of FIGS. 1 to 33 or to a polynucleotide from the coding sequence of a gene which encompasses a gene fragment according to any of FIGS. 1 to 33.

15. Polynucleotide according to claim 14, characterised in that it corresponds by at least 90%, preferably 95%, in particular at least 97% to any of the nucleotide sequences represented in any of FIGS. 1, 3-8, 10, 11, 13, 14, 15, 17-27 or 31-33, in particular 20 or 21, or to parts thereof, or to a polynucleotide from the coding sequence of a gene which encompasses a gene fragment according to any of FIGS. 1, 3-8, 10, 11, 13, 14, 15, 17-27 or 31-33, in particular 20 or 21.

16. Polynucleotide which corresponds by at least 90%, preferably 95%, in particular by at least 97% to a defined nucleic acid sequence obtainable by labelling a polynucleotide according to any of FIGS. 1 to 33 as a probe, hybridising a cDNA bank with the probe and washing under stringent conditions, and isolating and optionally sequencing the cDNA clone to which the probe has bound.

17. Polynucleotide which corresponds by at least 90%, preferably 95%, in particular by at least 97% to a defined nucleic acid sequence obtainable by determining and synthesising segments of a polynucleotide according to any of FIGS. 1 to 33 as gene-specific oligonucleotide primers, with which the extended polynucleotide is then generated and optionally sequenced by means of PCR using single or double-stranded DNA, cDNA libraries or genomic DNA as a template.

18. Polynucleotide according to either of claims 16 or 17, characterised in that a polynucleotide according to any of FIGS. 1, 3-8, 10, 11, 13, 14, 15, 17-27 or 31-33, in particular 20 or 21, is used.

19. Polynucleotide according to any of claims 14 to 18, characterised in that it is RNA or single or double-stranded DNA, in particular mRNA or cDNA.

20. Polynucleotide according to any of claims 14 to 19, characterised in that it is an antisense polynucleotide or PNA showing a sequence which is capable of specifically binding to a polynucleotide according to any of claims 14 to 19.

21. Polynucleotide according to claim 20, characterised in that it is part of a ribozyme or other DNA enzyme or of a catalytic RNA or DNA.

22. Vector containing a polynucleotide according to any of claims 14 to 21.

23. Vector according to claim 22, characterised in that it is an expression vector.

24. Vector according to either of claims 22 or 23, characterised in that the vector is derived from a virus, for example the adenovirus, adeno-associated virus or herpes virus, and/or that it contains at least one LTR, poly A, promoter and/or ORI sequence.

25. Vector according to any of claims 22 to 24, characterised in that it contains a polynucleotide according to either of claims 15 or 18.

26. Peptide, in particular oligopeptide or polypeptide, or protein encoded by a polynucleotide according to any of claims 14-19.

27. Peptide, in particular oligopeptide or polypeptide, or protein which is encoded by a polynucleotide that, under stringent conditions, hybridises with any of the polynucleotides according to FIGS. 1 to 33 or with any of the antisense polynucleotides thereof.

28. Peptide or protein according to either of claims 26 or 27, characterised in that it was modified after translation, in particular glycosylated, phosphorylated, amidated, methylated, acetylated, ADP ribosylated, hydroxylated, provided with a membrane anchor, cleaved or shortened.

29. Peptide or protein according to any of claims 26 to 28, characterised in that it is encoded by a polynucleotide according to either of claims 15 or 18.

30. Antibody for a peptide or protein according to any of claims 26 to 29.

31. Antibody according to claim 30, characterised in that it is a monoclonal or polyclonal antibody.

32. Antibody according to either of claims 30 or 31, characterised in that it is an antibody for a peptide or protein according to claim 29.

33. Cell containing a polynucleotide according to any of claims 14 to 21, a peptide or protein according to any of claims 26 to 29 and/or a vector according to any of claims 22 to 25.

34. Cell according claim 33, characterised in that the cell is an amphibian cell, a bacterial cell, a yeast cell, an insect cell or an immortalised or native mammalian cell.

35. Cell according to either of claims 33 or 34, characterised in that it contains a nucleic acid sequence according to claim 15 or 18, a peptide or protein according to claim 29 and/or a vector according to claim 25.

36. Transgenic non-human mammal, whose germ and somatic cells contain a nucleotide sequence according to any of claims 14 to 21 as the result of introducing a chromosome or chromosomes into the genome of the animal or into the genome of any of the ancestors of said animal.

37. Transgenic non-human mammal, whose embryonic and somatic cells contain any of the nucleotide sequences according to any of claims 14 to 21 as the result of chromosomal manipulation in the genome of the animal or in the genome of any of the ancestors of said animal, the sequence no longer being in expressible form.

38. Transgenic non-human mammal, according to either of claims 36 or 37, characterised in that it is a rodent.

39. Transgenic non-human mammal, according to any of claims 36 to 38, characterised in that the nucleotide sequence corresponds to claim 15 or 18.

40. Compound identifiable as a pain-regulating substance by means of a method according to any of claims 1 to 13.

41. Compound according to claim 40, characterised in that the compound is identifiable as a pain-regulating substance by means of a method according to claim 11.

42. Compound according to claim 40, characterised in that the compound is identifiable as a pain-regulating substance by means of a method according to claim 12.

43. Compound according to claim 40, characterised in that the compound is identifiable as a pain-regulating substance by means of a method according to claim 13.

44. Active ingredient characterised in that it binds to a peptide or protein according to any of claims 26 to 29.

45. Active ingredient according to claim 44, characterised in that it is a low-molecular active ingredient.

46. Active ingredient according to either of claims 44 or 45, characterised in that it binds to a peptide or protein according to claim 29.

47. Medicament containing at least one polynucleotide according to any of claims 14-21, a peptide or protein according to any of claims 26 to 29, a vector according to any of claims 22 to 25, an antibody according to any of claims 30 to 32, a cell according to any of claims 33 to 35, a compound according to any of claims 40 to 43 and/or an active ingredient according to any of claims 44 to 46, and, optionally, appropriate auxiliary and/or supplementary ingredients.

48. Medicament according to claim 47, characterised in that it contains a polynucleotide according to either of claims 15 or 18, a peptide or protein according to claim 29, a vector according to claim 25, an antibody according to claim 32, a cell according to claim 35, a compound according to either of claims 41 or 42 and/or an active ingredient according to claim 46.

49. Medicament according to either of claims 47 or 48, characterised in that it contains a compound according to claim 41.

50. Medicament according to either of claims 47 or 48, characterised in that it contains a compound according to claim 42 and/or an active ingredient according to claim 46.

51. Diagnostic reagent containing at least one polynucleotide according to any of claims 14-21 or parts thereof a peptide or protein according to any of claims 26 to 29 or parts thereof a vector according to any of claims 22 to 25, an antibody according to any of claims 30 to 32 or parts thereof and/or a cell according to any of claims 33 to 35 and, optionally, appropriate supplementary ingredients.

52. Diagnostic reagent according to claim 51, characterised in that it contains a polynucleotide according to either of claims 15 or 18 or parts thereof, a peptide or protein according to claim 29 or parts thereof, a vector according to claim 25, an antibody according to claim 32 and/or a cell according to claim 35.

53. Diagnostic reagent according to either of claims 51 or 52, characterised in that it contains a polynucleotide according to claim 20.

54. Use of a polynucleotide according to any of claims 14 to 21, a peptide or protein according to any of claims 26-29, a vector according to any of claims 22-25, an antibody according to any of claims 30-32, a cell according to any of claims 33-35, a compound according to any of claims 40-43, an active ingredient according to any of claims 44-46 and/or an active ingredient which binds to a peptide or protein selected from the following group:
JNK3, PIM-2, LR-11, TFIIFβ, GGT-β, GATA3, CLC-7, catalase, tetraspanin (TM4SF/TSPAN-6/TM4-D), casein kinase 1a, MAP3K7/TAK-1 (TGF-β activated kinase), BAB14112 and/or spermidine synthase,
a peptide or protein which, or a part of which, is encoded by a polynucleotide according to any of FIGS. 34a), 34c), 34e), 34g), 35a), 35c), 35e), 36a), 36c), 37a), 37c), 38a), 38c), 39a), 39c), 39e), 40a), 40c), 40e), 41a), 41c), 41e), 42a), 42e), 43c), 43e), 44e), 45a), 45c), 45e), or 46e) or which is encoded by a polynucleotide which is at least 90%, preferably 95%, in particular 97% similar thereto, or
a peptide or protein having an amino acid sequence according to any of FIGS. 34b), 34d), 34f), 34h), 35b), 35d), 35f), 36b), 36d), 37b), 37d), 38b), 38d), 39b), 39d), 40b), 40d), 40f), 41b), 41d), 41f), 42b), 42f), 43d), 43f), 44f), 45b), 45d), 45f) or 46f), or a peptide or protein at least 90%, preferably 95%, in particular 97% similar thereto
and/or a protein, which is encoded by a nucleic acid which, under stringent conditions, binds to a polynucleotide according to any of FIGS. 34a), 34c), 34e), 34g), 35a), 35c), 35e), 36a), 36c), 37a), 37c), 38a), 38c), 39a), 39c), 39e), 40a), 40c), 40e), 41a), 41c), 41e), 42a), 42e), 43c), 43e), 44e), 45a), 45c), 45e), or 46e) or to the antisense polynucleotide thereof and/or
a partial protein, at least 10, preferably 15, in particular 20 amino acids long, of one of the aforementioned proteins and/or peptides
for producing a medicament for the treatment of pain.

55. Use according to claim 54, characterised in that the treatment relates to chronic pain.

56. Use according to either of claims 54 or 55, characterised in that a polynucleotide according to either of claims 15 or 18, a peptide or protein according to claim 29, a vector according to claim 25, an antibody according to claim 32, a cell according to claim 35, a compound according to either of claims 41 or 42, an active ingredient according to claim 46 and/or an active ingredient which binds to a peptide or a protein selected from the following group is used:
JNK3, PIM-2, LR-11, TFIIFβ, GGT-β, GATA3, CLC-7, catalase, tetraspanin (TM4SF/TSPAN-6/TM4-D), casein kinase 1a, MAP3K7/TAK-1 (TGF-β activated kinase), BAB14112 and/or spermidine synthase,
a peptide or protein for which, or for a part of which a polynucleotide codes according to any of FIGS. 34a), 34c), 34e), 34g), 35a), 35c), 35e), 36a), 36c), 37a), 37c), 38a), 38c), 39a), 39c), 39e), 40a), 40c), 40e), 41a), 41c), 41e), 42a), 42e), 43c), 43e), 44e), 45a), 45c), 45e), or 46e), or a polynucleotide at least 90%, preferably 95%, in particular 97% similar thereto, or
a peptide or a protein having an amino acid sequence according to any of FIGS. 34b), 34d), 34f), 34h), 35b), 35d), 35f), 36b), 36d), 37b), 37d), 38b), 38d), 39b), 39d), 40b), 40d), 40f), 41b), 41d), 41f), 42b), 42f), 43d), 43f), 44f), 45b), 45d), 45f) or 46f), or a peptide or protein at least 90%, preferably 95%, in particular 97% similar thereto.

57. Use according to any of claims 54-56, characterised in that a compound according to claim 41 and/or an active ingredient which binds to a peptide or a protein selected from the following group is used:
JNK3, PIM-2, LR-11, TFIIFβ, GGT-β, GATA3, CLC-7, catalase, tetraspanin (TM4SF/TSPAN-6/TM4-D), casein kinase 1a, MAP3K7/TAK-1 (TGF-β activated kinase), BAB14112 and/or spermidine synthase,
a peptide or protein which, or a part of which, is encoded by a polynucleotide according to any of FIGS. 34a), 34c), 34e), 34g), 35a), 35c), 35e), 36a), 36c), 37a), 37c), 38a), 38c), 39a), 39c), 39e), 40a), 40c), 40e), 41a), 41c), 41e), 42a), 42e), 43c), 43e), 44e), 45a), 45c), 45e), or 46e), or a polynucleotide at least 90%, preferably 95%, in particular 97% similar thereto, or
a peptide or a protein having an amino acid sequence according to any of FIGS. 34b), 34d), 34f), 34h), 35b), 35d), 35f), 36b), 36d), 37b), 37d), 38b), 38d), 39b), 39d), 40b), 40d), 40f), 41b), 41d), 41f), 42b), 42f), 43d), 43f), 44f), 45b), 45d), 45f) or 46f), or a peptide or protein at least 90%, preferably 95%, in particular 97% similar thereto.

58. Use according to any of claims 54-56, characterised in that a compound according to claim 42 and/or an active ingredient according to claim 46 is used.

59. Use of a polynucleotide according to any of claims 14 to 21, a peptide or protein according to any of claims 26-29, a vector according to any of claims 22-25, an antibody according to any of claims 30-32, and/or a cell according to any of claims 33-35 for gene therapy.

60. Use according to claim 59, characterised in that it is in-vivo or in-vitro gene therapy.

61. Use according to either of claims 59 or 60, characterised in that a polynucleotide according to any of claims 16-18, preferably 18 is used.

62. Use according to either of claims 59 or 60, characterised in that a polynucleotide according to claim 15 is used.

63. Use according to either of claims 59 or 60, characterised in that a polynucleotide according to either of claims 20 or 21 is used.

64. Use of a polynucleotide according to any of claims 14 to 21, a peptide or protein according to any of claims 26-29, a vector according to any of claims 22-25, an antibody according to any of claims 30-32, a cell according to any of claims 33-35, a compound according to any of claims 40-43 and/or an active ingredient according to any of claims 44-46 for diagnosis and/or for studies of effectiveness.

65. Method for discovering pain-regulating substances having the following process steps:
(a) Incubation of a substance to be tested under appropriate conditions with a cell and/or a preparation from such a cell which has synthesised a peptide or protein which is encoded by a polynucleotide according to any of claims 16-18, preferably 18, or a polynucleotide which is at least 90%, preferably 95%, in particular 97% similar thereto,
(b) measurement of the binding of the test substance to the peptide or protein synthesised by the cell or measurement of at least one of the functional parameters altered by the binding of the test substance to the peptide or protein.

66. Method according to claim 65, characterised in that the cell is genetically manipulated before step (a).

67. Method according to claim 66, characterised in that the genetic manipulation allows the measurement of at least one of the functional parameters altered by the test substance.

68. Method according to either of claims 66 or 67, characterised in that the cell is genetically manipulated in such a way that the cell contains at least one polynucleotide according to any of claims 15-18, preferably 18, or a polynucleotide at least 90%, preferably 95%, in particular 97% similar thereto.

69. Method according to any of claims 66 to 68, characterised in that after the genetic manipulation according to claim 66 and before step (a) according to claim 65 the cell is cultivated under conditions which allow expression, optionally under selection pressure.

70. Method for the production of a peptide or protein according to any of claims 26-29, characterised in that a cell is cultivated according to any of claims 33-35, and optionally the peptide or protein is isolated.

71. Use of a peptide or protein selected from any of the following groups:
JNK3, PIM-2, LR-11, TFIIFβ, GGT-β, GATA3, CLC-7, catalase, tetraspanin (TM4SF/TSPAN-6/TM4-D), casein kinase 1a, MAP3K7/TAK-1 (TGF-β activated kinase), BAB14112 and/or spermidine synthase,
a peptide or protein, which or a part of which is encoded by a polynucleotide according to any of FIGS. 34a), 34c), 34e), 34g), 35a), 35c), 35e), 36a), 36c), 37a), 37c), 38a), 38c), 39a), 39c), 39e), 40a), 40c), 40e), 41a), 41c), 41e), 42a), 42e), 43c), 43e), 44e), 45a), 45c), 45e), or 46e) or which is encoded by a polynucleotide which is at least 90% similar thereto
a peptide or protein having an amino acid sequence according to any of FIGS. 34b), 34d), 34f), 34h), 35b), 35d), 35f), 36b), 36d), 37b), 37d), 38b), 38d), 39b), 39d), 40b), 40d), 40f), 41b), 41d), 41f), 42b), 42f), 43d), 43f), 44f), 45b), 45d), 45f) or 46f) or a peptide or protein at least 90% similar thereto
and/or a protein, which is encoded by a nucleic acid which, under stringent conditions, binds to a polynucleotide according to any of FIGS. 34a), 34c), 34e), 34g), 35a), 35c), 35e), 36a), 36c), 37a), 37c), 38a), 38c), 39a), 39c), 39e), 40a), 40c), 40e), 41a), 41c), 41e), 42a), 42e), 43c), 43e), 44e), 45a), 45c), 45e), or 46e) or to the antisense polynucleotide thereof,
a partial protein at least 10 amino acids long, of one or the aforementioned proteins and/or peptides and/or
a peptide or protein which is encoded by a gene consisting of a polynucleotide, whose nucleotide sequence encompasses a gene fragment according to any of the sequences represented in FIGS. 1 to 33, or which is encoded by a polynucleotide which is at least 90% similar to such a gene
in a method for discovering pain-regulating substances.
